SEQUENCE LISTING (23 sequences)

SEQ ID NO: 1 - DNA, 212 bp, synthetic construct
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 2 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 3 - DNA, 1965 bp, synthetic construct
  source 1..1965: mol_type = other DNA
ggtacccctattcaccgattttgattcccgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagatgctgctgctcgtgacatctctgctgctgtgcgagctgccccaccccgcctttctgctgatccccgatatcctgctgacccagacccctctgagcctgcctgtgtctctgggcgatcaggccagcatcagctgcagatccagccagagcctggtgcaccggaacggcaacacctacctgcactggtatctgcagaagcccggccagagccccaagctgctgattcacaaggtgtccaaccggttcagcggcgtgcccgacagattttctggcagcggctccggcaccgacttcaccctgaagatcagccgggtggaagccgaggacctgggcgtgtacttctgcagccagtccacccacgtgccccccctgacatttggcgccggaacaaagctggaactgaagggcagcacaagcggcagcggcaagcctggatctggcgagggaagcaccaagggcgaagtgaagctgcagcagagcggcccctctctggtggaacctggcgcctctgtgatgatctcctgcaaggccagcggcagctccttcaccggctacaacatgaactgggtgcgccagaacatcggcaagagcctggaatggatcggcgccatcgacccctactacggcggcaccagctacaaccagaagttcaagggcagagccaccctgaccgtggacaagagcagctccaccgcctacatgcacctgaagtccctgaccagcgaggacagcgccgtgtactactgcgtgtccggcatggaatactggggccagggcacaagcgtgaccgtgtcctctccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgttaagctagc

SEQ ID NO: 4 - DNA, 1988 bp, synthetic construct
  source 1..1988: mol_type = other DNA
ggtacccctattcaccgattttgattcccgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagatgctgctgctcgtgacatctctgctgctgtgcgagctgccccaccccgcctttctgctgatccccgatatcctgctgacccagacccctctgagcctgcctgtgtctctgggcgatcaggccagcatcagctgcagatccagccagagcctggtgcaccggaacggcaacacctacctgcactggtatctgcagaagcccggccagagccccaagctgctgattcacaaggtgtccaaccggttcagcggcgtgcccgacagattttctggcagcggctccggcaccgacttcaccctgaagatcagccgggtggaagccgaggacctgggcgtgtacttctgcagccagtccacccacgtgccccccctgacatttggcgccggaacaaagctggaactgaagggcagcacaagcggcagcggcaagcctggatctggcgagggaagcaccaagggcgaagtgaagctgcagcagagcggcccctctctggtggaacctggcgcctctgtgatgatctcctgcaaggccagcggcagctccttcaccggctacaacatgaactgggtgcgccagaacatcggcaagagcctggaatggatcggcgccatcgacccctactacggcggcaccagctacaaccagaagttcaagggcagagccaccctgaccgtggacaagagcagctccaccgcctacatgcacctgaagtccctgaccagcgaggacagcgccgtgtactactgcgtgtccggcatggaatactggggccagggcacaagcgtgaccgtgtcctctccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgttaacctattcaccgattttgattcccgctagc

SEQ ID NO: 5 - DNA, 2759 bp, synthetic construct
  source 1..2759: mol_type = other DNA
ggtacccctattcaccgattttgattccctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctttaacgcggccgctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagttagaattcttattagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgctggtacgcgggggcgtctgcgctcctgctgaacttcactctcagttcacatcctccttcttcttcttctggaaatcggcagctacagccatcttcctcttgagtagtttgtactggtctcataaatggttgtttgaatatatacaggagtttctttctgccccgtttgcagtaaagggtgataaccagtgacaggagaaggaccccacaagtcccggccaagggcgcccagatgtagatatcacaggcgaagtccagccccctcgtgtgcactgcgccccccgccgctggccggcacgcctctgggcgcagggacaggggctgcgacgcgatggtgggcgccggtgttggtggtcgcggcgctggagaggacacggtcacgcttgtgccctggccccagtattccatgccggacacgcagtagtacacggcgctgtcctcgctggtcagggacttcaggtgcatgtaggcggtggagctgctcttgtccacggtcagggtggctctgcccttgaacttctggttgtagctggtgccgccgtagtaggggtcgatggcgccgatccattccaggctcttgccgatgttctggcgcacccagttcatgttgtagccggtgaaggagctgccgctggccttgcaggagatcatcacagaggcgccaggttccaccagagaggggccgctctgctgcagcttcacttcgcccttggtgcttccctcgccagatccaggcttgccgctgccgcttgtgctgcccttcagttccagctttgttccggcgccaaatgtcagggggggcacgtgggtggactggctgcagaagtacacgcccaggtcctcggcttccacccggctgatcttcagggtgaagtcggtgccggagccgctgccagaaaatctgtcgggcacgccgctgaaccggttggacaccttgtgaatcagcagcttggggctctggccgggcttctgcagataccagtgcaggtaggtgttgccgttccggtgcaccaggctctggctggatctgcagctgatgctggcctgatcgcccagagacacaggcaggctcagaggggtctgggtcagcaggatatcggggatcagcagaaaggcggggtggggcagctcgcacagcagcagagatgtcacgagcagcagcatctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgccctttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacctattcaccgattttgattcccgctagc

SEQ ID NO: 6 - DNA, 2580 bp, synthetic construct
  source 1..2580: mol_type = other DNA
ggtacccctattcaccgattttgattcccgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagatgctgctgctcgtgacatctctgctgctgtgcgagctgccccaccccgcctttctgctgatccccgatatcctgctgacccagacccctctgagcctgcctgtgtctctgggcgatcaggccagcatcagctgcagatccagccagagcctggtgcaccggaacggcaacacctacctgcactggtatctgcagaagcccggccagagccccaagctgctgattcacaaggtgtccaaccggttcagcggcgtgcccgacagattttctggcagcggctccggcaccgacttcaccctgaagatcagccgggtggaagccgaggacctgggcgtgtacttctgcagccagtccacccacgtgccccccctgacatttggcgccggaacaaagctggaactgaagggcagcacaagcggcagcggcaagcctggatctggcgagggaagcaccaagggcgaagtgaagctgcagcagagcggcccctctctggtggaacctggcgcctctgtgatgatctcctgcaaggccagcggcagctccttcaccggctacaacatgaactgggtgcgccagaacatcggcaagagcctggaatggatcggcgccatcgacccctactacggcggcaccagctacaaccagaagttcaagggcagagccaccctgaccgtggacaagagcagctccaccgcctacatgcacctgaagtccctgaccagcgaggacagcgccgtgtactactgcgtgtccggcatggaatactggggccagggcacaagcgtgaccgtgtcctctccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgaaggccgagggagcctgctgacatgtggcgatgtggaggaaaacccaggaccaatggtgggcagcctgaactgcatcgtggccgtgagccagaacatgggcatcggcaagaacggcgacttcccctggccccccctgagaaacgagagcagatacttccagagaatgaccaccaccagcagcgtggagggcaagcagaacctggtgatcatgggcaagaagacctggttcagcatccccgagaagaacagacccctgaagggcagaatcaacctggtgctgagcagagagctgaaggagcccccccagggcgcccacttcctgagcagaagcctggacgacgccctgaagctgaccgagcagcccgagctggccaacaaggtggacatggtgtggatcgtgggcggcagcagcgtgtacaaggaggccatgaaccaccccggccacctgaagctgttcgtgaccagaatcatgcaggacttcgagagcgacaccttcttccccgagatcgacctggagaagtacaagctgctgcccgagtaccccggcgtgctgagcgacgtgcaggaggagaagggcatcaagtacaagctggaggtgtacgagaagaacgactaataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgttaagctagc

SEQ ID NO: 7 - DNA, 3087 bp, synthetic construct
  source 1..3087: mol_type = other DNA
ggtacccctattcaccgattttgattcccgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagatgctgctgctcgtgacatctctgctgctgtgcgagctgccccaccccgcctttctgctgatccccgatatcctgctgacccagacccctctgagcctgcctgtgtctctgggcgatcaggccagcatcagctgcagatccagccagagcctggtgcaccggaacggcaacacctacctgcactggtatctgcagaagcccggccagagccccaagctgctgattcacaaggtgtccaaccggttcagcggcgtgcccgacagattttctggcagcggctccggcaccgacttcaccctgaagatcagccgggtggaagccgaggacctgggcgtgtacttctgcagccagtccacccacgtgccccccctgacatttggcgccggaacaaagctggaactgaagggcagcacaagcggcagcggcaagcctggatctggcgagggaagcaccaagggcgaagtgaagctgcagcagagcggcccctctctggtggaacctggcgcctctgtgatgatctcctgcaaggccagcggcagctccttcaccggctacaacatgaactgggtgcgccagaacatcggcaagagcctggaatggatcggcgccatcgacccctactacggcggcaccagctacaaccagaagttcaagggcagagccaccctgaccgtggacaagagcagctccaccgcctacatgcacctgaagtccctgaccagcgaggacagcgccgtgtactactgcgtgtccggcatggaatactggggccagggcacaagcgtgaccgtgtcctctccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgaaggccgagggagcctgctgacatgtggcgatgtggaggaaaacccaggaccaatgcttctcctggtgacaagccttctgctctgtgagttaccacacccagcattcctcctgatcccacgcaaagtgtgtaacggaataggtattggtgaatttaaagactcactctccataaatgctacgaatattaaacacttcaaaaactgcacctccatcagtggcgatctccacatcctgccggtggcatttaggggtgactccttcacacatactcctcctctggatccacaggaactggatattctgaaaaccgtaaaggaaatcacagggtttttgctgattcaggcttggcctgaaaacaggacggacctccatgcctttgagaacctagaaatcatacgcggcaggaccaagcaacatggtcagttttctcttgcagtcgtcagcctgaacataacatccttgggattacgctccctcaaggagataagtgatggagatgtgataatttcaggaaacaaaaatttgtgctatgcaaatacaataaactggaaaaaactgtttgggacctccggtcagaaaaccaaaattataagcaacagaggtgaaaacagctgcaaggccacaggccaggtctgccatgccttgtgctcccccgagggctgctggggcccggagcccagggactgcgtctcttgccggaatgtcagccgaggcagggaatgcgtggacaagtgcaaccttctggagggtgagccaagggagtttgtggagaactctgagtgcatacagtgccacccagagtgcctgcctcaggccatgaacatcacctgcacaggacggggaccagacaactgtatccagtgtgcccactacattgacggcccccactgcgtcaagacctgcccggcaggagtcatgggagaaaacaacaccctggtctggaagtacgcagacgccggccatgtgtgccacctgtgccatccaaactgcacctacggatgcactgggccaggtcttgaaggctgtccaacgaatgggcctaagatcccgtccatcgccactgggatggtgggggccctcctcttgctgctggtggtggccctggggatcggcctcttctaataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgttaagctagc

SEQ ID NO: 8 - DNA, 2841 bp, synthetic construct
  source 1..2841: mol_type = other DNA
ggtacccctattcaccgattttgattcccgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagatgctgctgctcgtgacatctctgctgctgtgcgagctgccccaccccgcctttctgctgatccccgatatcctgctgacccagacccctctgagcctgcctgtgtctctgggcgatcaggccagcatcagctgcagatccagccagagcctggtgcaccggaacggcaacacctacctgcactggtatctgcagaagcccggccagagccccaagctgctgattcacaaggtgtccaaccggttcagcggcgtgcccgacagattttctggcagcggctccggcaccgacttcaccctgaagatcagccgggtggaagccgaggacctgggcgtgtacttctgcagccagtccacccacgtgccccccctgacatttggcgccggaacaaagctggaactgaagggcagcacaagcggcagcggcaagcctggatctggcgagggaagcaccaagggcgaagtgaagctgcagcagagcggcccctctctggtggaacctggcgcctctgtgatgatctcctgcaaggccagcggcagctccttcaccggctacaacatgaactgggtgcgccagaacatcggcaagagcctggaatggatcggcgccatcgacccctactacggcggcaccagctacaaccagaagttcaagggcagagccaccctgaccgtggacaagagcagctccaccgcctacatgcacctgaagtccctgaccagcgaggacagcgccgtgtactactgcgtgtccggcatggaatactggggccagggcacaagcgtgaccgtgtcctctccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgaaggccgagggagcctgctgacatgtggcgatgtggaggaaaacccaggaccaatgggggcaggtgccaccggccgcgcaatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtgataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgttaagctagc

SEQ ID NO: 9 - DNA, 3006 bp, synthetic construct
  source 1..3006: mol_type = other DNA
ggtacccccacagatatccagaaccctgacctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacaagcttctgacctcttctcttcctcccacagggcctcgagagatctggcagcggagaaggccgagggagcctgctgacatgtggcgatgtggaggaaaacccaggaccaccatggatgctgctgctcgtgacatctctgctgctgtgcgagctgccccaccccgcctttctgctgatccccgatatcctgctgacccagacccctctgagcctgcctgtgtctctgggcgatcaggccagcatcagctgcagatccagccagagcctggtgcaccggaacggcaacacctacctgcactggtatctgcagaagcccggccagagccccaagctgctgattcacaaggtgtccaaccggttcagcggcgtgcccgacagattttctggcagcggctccggcaccgacttcaccctgaagatcagccgggtggaagccgaggacctgggcgtgtacttctgcagccagtccacccacgtgccccccctgacatttggcgccggaacaaagctggaactgaagggcagcacaagcggcagcggcaagcctggatctggcgagggaagcaccaagggcgaagtgaagctgcagcagagcggcccctctctggtggaacctggcgcctctgtgatgatctcctgcaaggccagcggcagctccttcaccggctacaacatgaactgggtgcgccagaacatcggcaagagcctggaatggatcggcgccatcgacccctactacggcggcaccagctacaaccagaagttcaagggcagagccaccctgaccgtggacaagagcagctccaccgcctacatgcacctgaagtccctgaccagcgaggacagcgccgtgtactactgcgtgtccggcatggaatactggggccagggcacaagcgtgaccgtgtcctctgcggccgcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaaacgcgtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttacccacagatatccagaaccctgagctagc

SEQ ID NO: 10 - DNA, 2768 bp, synthetic construct
  source 1..2768: mol_type = other DNA
ggtacccccacagatatccagaaccctgacctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacattaacgcggccgctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagttagaattcttattagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgctggtacgcgggggcgtctgcgctcctgctgaacttcactctcagttcacatcctccttcttcttcttctggaaatcggcagctacagccatcttcctcttgagtagtttgtactggtctcataaatggttgtttgaatatatacaggagtttctttctgccccgtttgcagtaaagggtgataaccagtgacaggagaaggaccccacaagtcccggccaagggcgcccagatgtagatatcacaggcgaagtccagccccctcgtgtgcactgcgccccccgccgctggccggcacgcctctgggcgcagggacaggggctgcgacgcgatggtgggcgccggtgttggtggtcgcggcgctggagaggacacggtcacgcttgtgccctggccccagtattccatgccggacacgcagtagtacacggcgctgtcctcgctggtcagggacttcaggtgcatgtaggcggtggagctgctcttgtccacggtcagggtggctctgcccttgaacttctggttgtagctggtgccgccgtagtaggggtcgatggcgccgatccattccaggctcttgccgatgttctggcgcacccagttcatgttgtagccggtgaaggagctgccgctggccttgcaggagatcatcacagaggcgccaggttccaccagagaggggccgctctgctgcagcttcacttcgcccttggtgcttccctcgccagatccaggcttgccgctgccgcttgtgctgcccttcagttccagctttgttccggcgccaaatgtcagggggggcacgtgggtggactggctgcagaagtacacgcccaggtcctcggcttccacccggctgatcttcagggtgaagtcggtgccggagccgctgccagaaaatctgtcgggcacgccgctgaaccggttggacaccttgtgaatcagcagcttggggctctggccgggcttctgcagataccagtgcaggtaggtgttgccgttccggtgcaccaggctctggctggatctgcagctgatgctggcctgatcgcccagagacacaggcaggctcagaggggtctgggtcagcaggatatcggggatcagcagaaaggcggggtggggcagctcgcacagcagcagagatgtcacgagcagcagcatggtggcctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttacccacagatatccagaaccctgagctagc

SEQ ID NO: 11 - DNA, 2762 bp, synthetic construct
  source 1..2762: mol_type = other DNA
ggtacccctattcaccgattttgattccccctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacattaacgcggccgctccccagcatgcctgctattctcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagttagaattcttattagcgagggggcagggcctgcatgtgaagggcgtcgtaggtgtccttggtggctgtactgagaccctggtaaaggccatcgtgccccttgcccctccggcgctcgcctttcatcccaatctcactgtaggcctccgccatcttatctttctgcagttcattgtacaggccttcctgagggttcttccttctcggctttccccccatctcagggtcccggccacgtctcttgtccaaaacatcgtactcctctcttcgtcctagattgagctcgttatagagctggttctggccctgctggtacgcgggggcgtctgcgctcctgctgaacttcactctcagttcacatcctccttcttcttcttctggaaatcggcagctacagccatcttcctcttgagtagtttgtactggtctcataaatggttgtttgaatatatacaggagtttctttctgccccgtttgcagtaaagggtgataaccagtgacaggagaaggaccccacaagtcccggccaagggcgcccagatgtagatatcacaggcgaagtccagccccctcgtgtgcactgcgccccccgccgctggccggcacgcctctgggcgcagggacaggggctgcgacgcgatggtgggcgccggtgttggtggtcgcggcgctggagaggacacggtcacgcttgtgccctggccccagtattccatgccggacacgcagtagtacacggcgctgtcctcgctggtcagggacttcaggtgcatgtaggcggtggagctgctcttgtccacggtcagggtggctctgcccttgaacttctggttgtagctggtgccgccgtagtaggggtcgatggcgccgatccattccaggctcttgccgatgttctggcgcacccagttcatgttgtagccggtgaaggagctgccgctggccttgcaggagatcatcacagaggcgccaggttccaccagagaggggccgctctgctgcagcttcacttcgcccttggtgcttccctcgccagatccaggcttgccgctgccgcttgtgctgcccttcagttccagctttgttccggcgccaaatgtcagggggggcacgtgggtggactggctgcagaagtacacgcccaggtcctcggcttccacccggctgatcttcagggtgaagtcggtgccggagccgctgccagaaaatctgtcgggcacgccgctgaaccggttggacaccttgtgaatcagcagcttggggctctggccgggcttctgcagataccagtgcaggtaggtgttgccgttccggtgcaccaggctctggctggatctgcagctgatgctggcctgatcgcccagagacacaggcaggctcagaggggtctgggtcagcaggatatcggggatcagcagaaaggcggggtggggcagctcgcacagcagcagagatgtcacgagcagcagcatctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcaccggttcaattgccgacccctccccccaacttctcggggactgtgggcgatgtgcgctctgcccgatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttacctattcaccgattttgattcccgctagc

SEQ ID NO: 12 - DNA, 1965 bp, synthetic construct
  source 1..1965: mol_type = other DNA
ggtaccccacggagcgagacatctcggccgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagatgctgctgctcgtgacatctctgctgctgtgcgagctgccccaccccgcctttctgctgatccccgatatcctgctgacccagacccctctgagcctgcctgtgtctctgggcgatcaggccagcatcagctgcagatccagccagagcctggtgcaccggaacggcaacacctacctgcactggtatctgcagaagcccggccagagccccaagctgctgattcacaaggtgtccaaccggttcagcggcgtgcccgacagattttctggcagcggctccggcaccgacttcaccctgaagatcagccgggtggaagccgaggacctgggcgtgtacttctgcagccagtccacccacgtgccccccctgacatttggcgccggaacaaagctggaactgaagggcagcacaagcggcagcggcaagcctggatctggcgagggaagcaccaagggcgaagtgaagctgcagcagagcggcccctctctggtggaacctggcgcctctgtgatgatctcctgcaaggccagcggcagctccttcaccggctacaacatgaactgggtgcgccagaacatcggcaagagcctggaatggatcggcgccatcgacccctactacggcggcaccagctacaaccagaagttcaagggcagagccaccctgaccgtggacaagagcagctccaccgcctacatgcacctgaagtccctgaccagcgaggacagcgccgtgtactactgcgtgtccggcatggaatactggggccagggcacaagcgtgaccgtgtcctctccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaataagaattctaactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgttaagctagc

SEQ ID NO: 13 - RNA, 20 nt, synthetic construct
  source 1..20: mol_type = other RNA
gggaatcaaaatcggtgaat

SEQ ID NO: 14 - RNA, 20 nt, synthetic construct
  source 1..20: mol_type = other RNA
gagaatcaaaatcggtgaat

SEQ ID NO: 15 - RNA, 20 nt, synthetic construct
  source 1..20: mol_type = other RNA
tcagggttctggatatctgt

SEQ ID NO: 16 - RNA, 20 nt, synthetic construct
  source 1..20: mol_type = other RNA
cgcgagcacagctaaggcca

SEQ ID NO: 17 - RNA, 20 nt, synthetic construct
  source 1..20: mol_type = other RNA
gagtagcgcgagcacagcta

SEQ ID NO: 18 - RNA, 20 nt, synthetic construct
  source 1..20: mol_type = other RNA
ggccgagatgtctcgctccg

SEQ ID NO: 19 - DNA, 19 bp, synthetic construct
  source 1..19: mol_type = other DNA
actgttcagacagacggat

SEQ ID NO: 20 - DNA, 86 bp, synthetic construct
  source 1..86: mol_type = other DNA
tgacaagtctgtctgcctattaataggggtacctaatgatttttatcaaaatcattaagttaaggtagatacacatcttgtcatat

SEQ ID NO: 21 - DNA, 80 bp, synthetic construct
  source 1..80: mol_type = other DNA
gggttgaagagcccctgacacccgctacacgcgagacgggcccttagttttagccacttataagtggctaaaactaagag

SEQ ID NO: 22 - DNA, 74 bp, synthetic construct
  source 1..74: mol_type = other DNA
cccaacttctcggggactgtgggcgatgtgcgctctgcccgggaatcaaaatcggtgcaccgattttgattctc

SEQ ID NO: 23 - DNA, 61 bp, synthetic construct
  source 1..61: mol_type = other DNA
ccccatggattactaaaaatagttttagtaattcaattccatctatgtgtagaacagtata

(19) United States
(12) Patent Application Publication
(10) Pub. No.: US 2026/0201420 A1
(43) Pub. Date: Jul. 16, 2026

(54) HOMOLOGY-INDEPENDENT TARGETED DNA INSERTION IN HUMAN T CELLS

(71) Applicant: The Board of Trustees of The Leland Stanford Junior University, Stanford, CA (US)

(72) Inventors: Crystal L. Mackall, Stanford, CA (US); Steven A. Feldman, Stanford, CA (US); Hyatt Balke-Want, Cologne (DE); Andrew Guy Mancini, Utrecht (NL)

(73) Assignee: The Board of Trustees of the Leland Stanford Junior University, Stanford, CA (US)

(21) Appl. No.: 19/135,745

(22) PCT Filed: Dec. 14, 2023
(86) PCT No.: PCT/US2023/084048

§ 371 Date: Jun. 4, 2025

Related U.S. Application Data
(60) Provisional application No. 63/432,879, filed on Dec. 15, 2022.
(60) Provisional application No. 63/508,393, filed on Jun. 15, 2023.

(51) Int. Cl. C12N 15/90; A61K 40/11; A61K 40/31; A61K 40/42; A61P 35/00; C07K 16/30; C12N 5/0783; C12N 9/22; C12N 13/00; C12N 15/11; C12N 15/87

(52) U.S. Cl. CPC C12N 15/907; A61K 40/11; A61K 40/31; A61K 40/4258; A61P 35/00; C07K 16/3084; C12N 5/0636; C12N 9/226; C12N 13/00; C12N 15/11; C12N 15/87; C12N 2310/20; C12N 2501/06; C12N 2501/727; C12N 2510/00

(57) ABSTRACT

The present disclosure provides methods and compositions for site directed CAR integration in primary human T cells using DNA delivered via homology-independent targeted insertion (HITI). The methods provide higher cell yield compared to homology-directed repair (HDR) mediated gene insertion. Post-HITI CRISPR EnrichMENT (CEMENT) using GMP grade reagents enriched CAR T cells to approximately 80% purity, resulting in therapeutically relevant dose ranges of CAR+ T cells.

### CROSS-REFERENCE TO RELATED APPLICATIONS

This application claims priority to U.S. Provisional Application No. 63/432,879, filed Dec. 15, 2022, and U.S. Provisional Application No. 63/508,393, filed Jun. 15, 2023, the disclosures of which are herein incorporated by reference in their entirety for all purposes.

### STATEMENT AS TO RIGHTS TO INVENTIONS MADE UNDER FEDERALLY SPONSORED RESEARCH AND DEVELOPMENT

This invention was made with Government support under contracts 5P30CA124435 awarded by the National Institutes of Health. The Government has certain rights in this invention.

### BACKGROUND

Treatment with CAR T cells has been proven to be effective for the treatment of various hematological disease¹⁻³. This has not only resulted in approval of this novel drug class for B-cell malignancies but fueled extensive efforts to investigate the efficacy of CAR T cells for the treatment of solid tumors⁴⁻⁸. Production of CAR T cells has mainly relied on viral vectors for transgene delivery⁹; however, major bottlenecks for executing clinical proof-of-concept trials are long lead times for production of viral vectors, and the large costs associated with GMP manufacture of these reagents.¹⁰ Therefore, non-viral manufacturing of CAR T cells has become an active area of research¹¹⁻¹⁶. Lately, transposition using PiggyBac or SleepingBeauty transposon systems have been applied for manufacturing of non-viral CAR T cells resulting in random genomic integration of CAR transgenes^{11,12,16}. During the manufacturing process, CAR T cells that are generated via transposition are commonly expanded using irradiated PBMCs or artificial antigen presenting cells (aAPCs)^{11,12} resulting in a prolonged culture period and chronic antigen stimulation of the transposed cells which increases the risk of manufacturing an exhausted CAR T cell product¹⁷. Furthermore, recent results from the CARTELL trial, which used PiggyBac manufactured anti-CD19 CAR T cells to treat B-cell malignancies, showed two cases of CAR T cell induced lymphoma, raising safety concerns when applying transposition for cell therapeutic applications^{13,18}. In contrast, CRISPR/Cas9 enables site-directed modifications of the genome. For large transgene insertions into primary human T cells HDR is typically used with AAV6 being the most common gene delivery reagent^{19,20}. Electroporation of CRISPR/Cas9 Ribonucleoprotein (RNP) along with either linearized or plasmid DNA has been proposed for targeted transgene knock-in into T cells but is associated with low efficiency and/or yield due to impaired T cell viability and expansion post editing^{21,22}. Therefore, a fully non-viral CRISPR knock-in platform for clinical scale manufacturing of CAR T cells has yet to be established.

Non-homologous end joining (NHEJ) is the primary DNA repair pathway after double-stranded DNA (dsDNA) breaks and acts independent of the cell cycle state. DNA ends are ligated by Ligase IV after Ku proteins recruit nucleases to trim and polymerases to fill in gaps respectively²³. In comparison, HDR relics on the division of a cell to provide a sister-chromatid chromosome copy created during S phase, which serves as a template for gene correction after a dsDNA break of the respective locus²⁴. Because HDR is not the predominant DNA repair pathway after dsDNA breaks, NHEJ mediated homology-independent targeted insertion (HITI) has more recently been explored for large transgene insertions in both dividing and resting cells²⁵. In addition, HITI resulted in more efficient targeted knock in when compared to HDR in adherent cell lines and embryonic stem cells. This effect was even more pronounced when using transgenes of large size (>5 kb)^{26,27}. To this point, HITI has been pre-clinically applied for the treatment of retinitis pigmentosa, Mucopolysaccharidosis type VI and Adrenoleukodystrophy using either AAV6 or AAV9 for transgene delivery^{25,28,29}. Due to its cell cycle independent integration HITI has the potential to expand the CRISPR knock-in toolbox for somatic cell and gene therapy³⁰. However, HITI has never been explored for CRISPR knock-in into T cells.

### BRIEF SUMMARY

The instant disclosure provides methods and compositions that are useful for enriching for T cells that express a chimeric antigen receptor (CAR).

In one aspect, the disclosure provides a method for enriching for T cells that express a chimeric antigen receptor (CAR), where the nucleotide sequences encoding the CAR are inserted at a target genomic locus following homology-independent targeted insertion (HITI).

In some embodiments, the method comprises: transfecting a plurality of T cells with
(a) a linear nucleic acid molecule comprising:
   (i) an expression cassette comprising a promoter operably linked to a sequence encoding a CAR and a sequence encoding a selectable marker; and
   (ii) a single protospacer adjacent motif (PAM) sequence that binds a guide RNA-CRISPR/Cas9 ribonucleoprotein (RNP), wherein the PAM is located 5′ or 3′ of the expression cassette of (i); and
(b) a guide RNA-CRISPR/Cas9 ribonucleoprotein (RNP);
   wherein the nucleic acid molecule is inserted into a target site in the genome of the T cell via non-homologous end joining (NHEJ); and isolating T cells that express the selectable marker, wherein the number of CAR+ T cells is increased compared to T cells that do not express the selectable marker.

In some embodiments, the nucleic acid molecule does not comprise sequences that are homologous to genomic sequences at the target genomic locus.

In some embodiments, the single PAM is located 5′ of the expression cassette of (i).

In some embodiments, the nucleic acid molecule is inserted on the positive or negative strand of the target genomic locus.

In some embodiments, the nucleic acid molecule comprises a plasmid having a plasmid backbone of less than 500 bp. In some embodiments, the plasmid comprises an insert size of at least 2000 bp. In some embodiments, the plasmid comprises an insert size between 2000 and 5000 bp.

In some embodiments, the promoter is an EF-1α promoter or EF-1α short (EFS) promoter. In some embodiments, the promoter comprises SEQ ID NO:1.

In some embodiments, the T cells are activated T cells. In some embodiments, the T cells are activated prior to transfection with the nucleic acid molecule.

In some embodiments, the T cells are incubated with an inhibitor of homology directed repair (HDR). In some embodiments, the inhibitor of HDR is selected from the group consisting of an ATM/ATR kinase inhibitor, a Chk1/Chk2 inhibitor, a BRCA1 inhibitor, a Rad51 inhibitor, and combinations thereof. In some embodiments, the inhibitor of HDR is an ATM/ATR kinase inhibitor.

In some embodiments, the selectable marker is a protein expressed on the cell surface and expression of the selectable marker is detected by contacting an antibody to the selectable marker. In some embodiments, the protein expressed on the cell surface is tEGFR or tNGFR. In some embodiments, the selectable marker is a protein that confers resistance to a drug or compound.

In some embodiments, the selectable marker is DHFR-FS, and the T cells are cultured with methotrexate (MTX) for a period of time to reduce the number of cells that do not express DHFR-FS. In some embodiments, the T cells are cultured with MTX beginning on day 1 after transfection until day 5 after transfection. In some embodiments, the T cells are cultured with MTX beginning on day 1 after transfection until day 4 after transfection, followed by culturing the T cells without MTX for 3 to 7 days. In some embodiments, the T cells are cultured with MTX beginning on day 5 after transfection until day 12 after transfection. In some embodiments, the T cells are cultured with MTX beginning on day 1 after transfection until day 5 after transfection, and are then cultured without MTX until day 12 after transfection, and the yield of CAR+ T cells at day 12 is similar to the yield of CAR+ T cells cultured with MTX beginning on day 5 after transfection until day 12 after transfection. In some embodiments, selection with MTX results in a frequency of CAR+ T cells that is equal to or greater than 70%.

In some embodiments, the transfection comprises electroporation.

In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1. IGF1R. CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

In some embodiments, the nucleic acid molecule is integrated into the T cell receptor alpha constant (TRAC), beta-2-microglobulin (B2M), or adeno-associated virus integration site 1 (AAVS1) genomic locus.

In some embodiments, the T cell is a primary human T cell. In some embodiments, the T cells are unstimulated T cells.

In another aspect, the disclosure provides a method for treating a tumor in a subject, the method comprising administering to the subject an effective amount of a genetically modified T cell comprising a nucleic acid molecule comprising:
(i) an expression cassette comprising a promoter operably linked to a sequence encoding a chimeric antigen receptor (CAR) and a sequence encoding a selectable marker; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i);
   wherein the nucleic acid molecule is inserted into a target site in the genome of the T cell via non-homologous end joining (NHEJ), thereby treating the tumor.

In some embodiments, the nucleic acid molecule does not comprise sequences that are homologous to genomic sequences at the target genomic locus.

In some embodiments, the single PAM is located 5′ of the expression cassette of (i).

In some embodiments, the nucleic acid molecule comprises a plasmid having a plasmid backbone of less than 500 bp. In some embodiments, the plasmid comprises an insert size between 2000 and 5000 bp.

In some embodiments, the promoter is an EF-1α promoter or EF-1α short (EFS) promoter. In some embodiments, the promoter comprises SEQ ID NO: 1.

In some embodiments, the selectable marker is DHFR-FS, and the T cells are cultured with methotrexate (MTX) for a period of time to reduce the number of cells that do not express DHFR-FS.

In some embodiments, the T cells are cultured with MTX beginning on day 1 after transfection until day 4 after transfection, followed by culturing the T cells without MTX for 3 to 7 days. In some embodiments, the T cells are cultured with MTX beginning on day 7 after transfection until day 14 after transfection.

In some embodiments, the selectable marker is a protein expressed on the cell surface and expression of the selectable marker is detected by contacting an antibody to the selectable marker. In some embodiments, the protein expressed on the cell surface is tEGFR or tNGFR.

In some embodiments, the CAR binds to an antigen selected from the group consisting of Her-2, B7-H3, GPC2, GD2, CD19, CD20, CD22. MAGE. BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7. CD244. CA-125, ETA, CEA, CD52, MUC5AC, c-Met, FAB, WT-1, PSMA, AFP, BCMA, Mesothelin, GPC3, MUC1 and CTAG1B.

In some embodiments, the nucleic acid molecule is integrated into the T cell receptor alpha constant (TRAC), beta-2-microglobulin (B2M), or adeno-associated virus integration site 1 (AAVS1) genomic locus.

In some embodiments, the T cells are activated prior to transfection with the nucleic acid molecule.

In some embodiments, 1×10⁶ to 1×10⁹ CAR+ T cells are administered to the subject. In some embodiments, 0.1×10⁶ to 5×10⁶ CAR+ T cells/kg of the subject's weight are administered to the subject. In some embodiments, the CAR+ T cells are administered in one or more doses.

In another aspect, the disclosure provides a genetically modified T cell comprising a nucleic acid molecule comprising:
(i) an expression cassette comprising a promoter operably linked to a sequence encoding a CAR and a sequence encoding DHFR-FS; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i).

In some embodiments, the promoter is an EF-1α promoter or EF-1α short (EFS) promoter. In some embodiments, the promoter comprises SEQ ID NO:1.

In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

In some embodiments, the T cell further comprises a guide RNA and CRISPR/Cas9 nuclease.

In some embodiments, the nucleic acid molecule is integrated into the T cell receptor alpha constant (TRAC), beta-2-microglobulin (B2M), or adeno-associated virus integration site 1 (AAVS1) genomic locus.

In some embodiments, the T cell is a primary human T cell. In some embodiments, the T cell is an activated T cell. In some embodiments, the T cell is an unstimulated T cell.

In another aspect, the disclosure provides a pharmaceutical composition comprising a genetically modified T cell (or a plurality genetically modified T cells) described herein.

### BRIEF DESCRIPTION OF THE DRAWINGS

FIG. 1: Comparison of Homology-Directed Recombination (HDR) versus Homology-independent-targeted-insertion (HITI) for targeted knock-in of a GD2-CAR into TRAC. a, Schematic overview of workflow for experiments in b-d, and plasmid designs for knock-in templates. b-d, head-to-head comparison of constructs HDR2c, HITI2c and HITI1c. 5×10⁶ cells were electroporated per condition on day 2 post activation using respective constructs (0.75 g of plasmid per 1×10⁶ cells) and analyzed via flow cytometry on day 10 (b, representative donor; c, pooled frequencies) and counted on the same day to assess GD2-CAR-T cell counts (d) (n=4 independent donors). e-h. HDR inhibitor induced modulation of GD2-CAR-T cell integration via HITI and HDR. e, Schematic related to f-h. f, Representative histograms of GD2-CAR expression after CRISPR knock-in with HDR2c or HITI1c templates either left untreated or treated with 1 μM of AZD0156 for 18 hours post electroporation. g+h, GD2-CAR expression (g) and GD2-CAR-T cell counts (h) normalized to untreated CRISPR knock-in samples after 18 hours of treatment with indicated concentrations of AZD0156 (n=3 independent donors). i-k, CRISPR knock-in of non-activated T cells using HITI1c and HDR2c for knock-in of the GD2-CAR. Indicated are knock-in frequencies (i), viability (j) and GD2-CAR-T cell yield (k). Cells were counted and analyzed via flow cytometry on day 10 (n=4 independent donors). p values were determined by paired two-tailed t tests. Error bars indicate standard deviation (SD).

FIG. 2: Optimization of Methotrexate (MTX) based selection of CRISPR knock-in GD2-CAR-DHFR-FS T cells. a, GD2-CAR-DHFR-FS plasmid design incorporating a gRNA cut site for linearization of the plasmid and dsDNA break in TRAC with correct transgene insertion indicated. b, Experimental layout for optimization of MTX enrichment. MTX treatment from day 7-14 has previously been reported to result in efficient enrichment in viral transduced CAR-DHFR-FS T cells and served as a reference. c, Titration of MTX in primary human T cells with efficient killing starting at 50 nM MTX (n=2 independent donors analyzed in technical duplicates). d. Comparison of knock-in frequency determined via flow cytometry on day 14 in GD2-CAR-DHFR-FS T cells either non-enriched, enriched from day 3-10 or from day 7-14 (n=5 independent donors). e, MTX time course after CRISPR knock-in starting on day 3 for up to 7 days with plateaued enrichment after 4 days of treatment. All samples were assessed via flow cytometry on day 14 (n=2 independent donors). f, Quadrant plots indicating TCR-a/b and GD2-CAR surface expression for two representatives out of five independent donors non-enriched, enriched from day 3-7 and from day 7-14. Flow cytometry was conducted on day 14. g, GD2-CAR-T cell yield at day 14. Fold changes were calculated based on number of activated T cells on day 2 (n=5 independent donors). Experiments in d and g were evaluated for statistical significance by paired, two-tailed t tests. Error bars indicate SD.

FIG. 3: HITI based CRISPR knock-in CAR-T cell manufacturing at clinical scale. a, Schematic workflow of leukapheresis processing to manufacture CRISPR KI CAR-T cells at clinical scale. Per Donor 1×10⁹ cells were activated and electroporated. Cultures were split up equally and either left untreated or treated with MTX for enrichment. b+c, Viability (b) and fold change (c) of respective cultures over time. d. Representative quadrant plots (day 14) showing GD2-CAR expression in TRAC positive cells for viral transduced CAR-T cells and TRAC negative cells for GD2 knock-in CAR-T cells. e, GD2-CAR frequency over time across all three donors. f, expansion of respective GD2-CAR-T cells for different time points normalized to the number of activated T cells. g, Total GD2-CAR-T cell counts for knock-in CAR-T cells (*=Donors with projected numbers after culture split on day 10). h, Frequency of viable and dead cells in edited and non-edited T cells after MTX treatment assessed via flow cytometry on day 7. All experiments were conducted with n=3 independent donors. Error bars indicate SD.

FIG. 4: Knock-in GD2-CAR-T cells do not show phenotypic differences and are not functionally inferior when compared to viral GD2-CAR-T cells. a, Changes of CD4/CD8 ratio after processing of leukopaks and over time. b+c. Memory marker (b) and exhaustion marker (c) expression of viral vs. GD2 knock-in CAR-T cells as determined via flow cytometry on day 14 (pooled data from n=3 independent donors). d, GD2 antigen levels of co-cultured tumor cell lines. Representative histograms from n=3 independent experiments. e. Intracellular cytokine (TNF-a, IL-2, IFN-g) and activation marker (CD107a, CD69) expression after 6 hours of co-culture with respective GD2 expressing tumor cell lines. Shown here is the marker positive cell frequency gated on CD8+ CAR+ T cells (pooled data from n=2 independent donors tested in technical triplicates). f, Concentration dependent tumor cell killing after 48 hours of co-culture with indicated E:T ratios (pooled data from n=3 independent donors). g, Tumor cell killing over time in GD2 antigen expressing tumor cell lines at a E:T ratio of 1:10 (pooled data from n=2 independent donors tested in technical triplicates). Error bars indicate SD.

FIG. 5: Knock-in GD2-CAR-T cells efficiently control growth of the SY5Y metastatic Neuroblastoma in vivo model. a, Schematic of SY5Y tumor cell injection (1×10⁶ on day 0 via tail vein injection) in NSG mice. Confirmed tumor engraftment on day 7 via bioluminescent imaging and consecutive GD2-CAR-T cell treatment on day 7 using 5×10⁶ GD2-CAR-T cells applied via tail vein injection followed by weekly imaging of tumor bioluminescence. b, Bioluminescent images of treated mice over time with color encoded radiance (p/sec/cm²/sr). c, Total flux values (p/s) of all animals over time. Statistical significance was evaluated using two-way ANOVA multiple comparisons along with Dunnett's test for indicated time points. d. Weight of all treated animals over time without relevant changes over baseline. e, Kaplan-Meier Survival analysis of treated animals. Statistical significance was evaluated using Mantel-Cox test. Error bars indicate SD.

FIG. 6: Genomic characterization of CRISPR knock-in CAR-T cells. a, On-target copy number estimation using ddPCR. Genomic DNA from scale up experiments (n=3 independent donors) was analyzed in technical duplicates using primers/probe to target Albumin (reference gene, copy number=2) and primers/probe to target the left insertion site of the GD2-CAR into TRAC. Copy number values were normalized to the frequency of CAR+ cells as determined via flow cytometry. b, Source of predicted off-target sites. c-e, Quantification of indels in predicted off-target sites and TRAC using CRISPAltRations for samples obtained from Donor 3 of large-scale experiments. Editing was binarily classified using a thresholded Fishers Exact test (p<0.05) with limitations (>0.5% indels in treatment; <0.4% indels in control: >5,000 reads) for edited samples with (c) knock-out, (d) knock-in without enrichment and (e) knock-in after enrichment (red circle=significant; blue circle=not significant). Indel frequencies were plotted against non-electroporated Mock control samples to highlight pre-existing indels and noise. Quadrants display the limits of classification (bottom left—treatment % indels<0.5; top right—control % indels>0.4%; top left—all limitations met and classifiable; bottom right—no limitations met). The top left quadrant contains classifiable events that occur in edited samples and indicates only on-target editing in these samples. f+g, Representative insertion site analysis for Donor 3 samples of non-enriched (f) and enriched (g) GD2 knock-in CAR-T cells using TLA. GD2 CAR sequences were inserted into the TRAC locus on chromosome 14 without evidence for off-target insertion.

FIG. 7: Design and concentration optimization of CRISPR knock-in plasmid constructs. a+b, Plasmid designs for HDR based GD2-CAR knock-in relying on the endogenous TRAC promoter for transgene expression (a) or on the exogenous EFS promoter (b) in reverse orientation. Both plasmid constructs have two gRNA cut sites. c, GD2-CAR knock-in frequency assessed via flow cytometry on day 7 after electroporation of 1×10⁶ cells on the Lonza 4D (day 2) using 0.75 μg of respective plasmid DNA. d, GD2-CAR-T cell yield on day 7 normalized to number of electroporated T cells. (n=2 independent donors). e+f, HDR plasmid optimization comparing 2 cut sites vs. uncut plasmid for CRISPR knock-in. Shown are knock-in frequencies (e) and yield (f) of knock-in CAR-T cells assessed on day 7 after electroporation of 5×10⁶ cells on day 2 using 0.75 μg of plasmid DNA per 1×10⁶ cells and the Maxcyte GTx (n=2 independent donors). Statistical significance assessed by paired, two-tailed t tests. g, Cell viability after electroporation with indicated amounts of plasmid per 1×10⁶ cells. h, GD2-CAR expression after electroporation with indicated amount of plasmid (n=2 independent donors). Error bars indicate SD.

FIG. 8: CRISPR knock-in using HITI1c integrates electroporated plasmid DNA and can be applied in a versatile manner. a, IN&OUT PCR of genomic DNA extracted from Mock, HITI1c and HDR2c samples using primers targeting the endogenous TRAC sequence outside homology arms of HDR2c and sequences within knock-in templates. b, Mapped Sanger Sequencing results from HITI1c after IN&OUT PCR from a showing minimal insertions at the left and minimal deletions at the right junction. c, Beta-2 Microglobulin surface expression after knock-out using three different gRNA constructs (n=2 independent donors). d-f, GD2-CAR knock-in into TRAC and B2M. Quadrant flow plots (d). GD2-CAR frequency (e) and yield (f) across three independent donors assessed on day 10. Error bars indicate SD.

FIG. 9: Optimization of surface-marker based enrichment after CRISPR knock-in. a+b, Comparison of post enrichment purity of GD2-CAR-tEGFR knock-in T cells using column free (Stemcell) and column based (Miltenyi) magnetic selection. CAR+ T cells were enriched on day 14. a, Representative quadrant flow plots from pre and post enrichment samples. b, Post enrichment Purity. c, Comparison of mid culture (day 9) and harvest (day 14) enrichment. Indicated are the pre and post enrichment purity of GD2-CAR-tEGFR knock-in T cells assessed on day 14. d, Post enrichment Viability (determined on day 14) for mid culture and harvest enriched GD2-CAR-tEGFR knock-in T cells. e, GD2-CAR-T cell yield normalized to electroporated number of T cells (day 14). f, Total T cell, and CAR+ T cell counts (day 14). All experiments were conducted with n=2 independent donors and d-f were analyzed using technical duplicates. Error bars indicate SD.

FIG. 10: Comparison of clinically established enrichment platforms reveals increased cell yields when DHFR-FS is knocked-in along with an GD2-CAR. a. GD2-CAR frequencies pre and post enrichment as determined via flow cytometry on day 14. GD2-CAR-tNGFR and GD2-CAR-tEGFR knock-in cells were enriched on day 9 using column based magnetic selection. b. Viability on day 14. c, total GD2-CAR-T cells counts on day 14 post enrichment. All experiments were conducted with n=2 independent donors. Differences were evaluated for statistical significance by paired, two-tailed t tests. Error bars indicate SD.

FIG. 11: Feasibility of HITI based CRISPR knock-in for clinical manufacturing. a, Enriched CD3+ T cell counts from adult and pediatric patient leukapheresis treated with viral CAR-T cells across different trials and manufactured at two sites. Dashed line indicates number of cells activated per condition (+/−MTX) in CRISPR knock-in scale up experiments. b, Day 2 T cells counts (post activation induced contraction) normalized to number of activated T cells for viral GD2-CAR-T cell manufacturing and our proposed CRISPR knock-in CAR-T cell manufacturing process. c, Overview of T cell numbers, reagent volumes and concentrations used for large scale electroporations. d+e, Post manufacturing viability (d) and normalized cell counts (e) of CRISPR knock-in CAR-T cells from non-enriched and MTX enriched donors (n=2 independent donors, counts from technical duplicates) either cultivated in media supplemented with IL-7/IL-15 or without cytokines. Statistical analysis performed with repeated measures ANOVA. Error bars indicate SD.

FIG. 12: Gating strategies. Gating strategies for CRISPR GD2-CAR knock-in T cells into TRAC (a), for viability of edited vs. non-edited T cells after knock-in GD2-CAR-DHFR-FS and MTX selection on day 7 (b), for phenotype, memory and exhaustion marker characterization (c) and activation marker and intracellular cytokines post co-culture with GD2 expressing cell lines (d).

FIG. 13: CAR and tumor antigen expression. CAR expression levels of viral transduced GD2-CAR and CRISPR knock-in GD2-CAR-DHFR-FS as indicated by histograms (a), median fluorescence intensity (MFI, b) and coefficient of variation (c) (n=3 independent donors from FIG. 3). Differences were evaluated for statistical significance by paired, two-tailed t tests. d, GD2 antigen levels on tumor cell lines described in FIG. 4. Molecule count/cell determined via Quantibrite beads (n=3 independent experiments). e+f, IL2 (e) and IFNg (f) secretion 24 hours after co-culture with respective GD2 antigen expressing tumor cell lines assessed via ELISA (n=2 independent donors, each donor was analyzed using technical triplicates). g, Intracellular cytokine (TNF-a, IL-2, IFN-g) and activation marker (CD107a, CD69) expression after 6 hours of co-culture with respective GD2 expressing tumor cell lines. Shown here is the marker positive cell frequency gated on CD4+ CAR+ T cells (n=2 independent donors, each donor was analyzed using technical triplicates). Error bars indicate SD.

FIG. 14: HITI/CEMENT enables CRISPR knock-in and enrichment of functional GPC2 knock-in CAR-T cells. a, Representative quadrant flow plot showing GPC2 CAR expression on day 10 in TRAC knock-out cells without MTX enrichment (left) or after enrichment in 50 nM MTX from day 3-7 (right). b, GPC2 CAR expression on day 10 after HITI mediated knock-in without (−MTX) or with enrichment (+MTX). c, GPC2 CAR yields on day 14 relative to electroporated cells (n=3 independent donors).

FIG. 15: rhAmpSeq sequencing quality control metrics and CRISPAltRations results for Donors 1 and 2. a, Genomic distribution of predicted off-target sites indicating no cutting within an Exon. b, Percentage of total reads that passed QC, were merged and mapped exceeded 95%, and primer dimers were found in <1% of samples. c, Total read counts per sample and d, per target indicating sufficient coverage. For OT 30 and 31 (both located on chromosome Y) results from the female Donor 1 were excluded from the analysis. e-g, CRISPAltRations results as shown in FIG. 6 c-e for Donors 1 and 2 of the large-scale experiments. Error bars indicate SD.

FIG. 16: TLA confirms on-target insertion. a-d, Genome-wide insertion site analysis indicates targeted insertion into TRAC locus at chromosome 14 across non-enriched (a+c) and enriched (b+d) samples from two additional, independent donors.

### DETAILED DESCRIPTION

### Introduction

The instant disclosure provides methods and compositions for producing chimeric antigen receptor (CAR) T cells using homology-independent targeted insertion (HITI). The methods and compositions provide the following advantages: i) production of autologous and fully non-viral transgenic CAR T cells at therapeutic doses; ii) a larger CAR transgene size than transgene sizes possible using homology directed repair (HDR); iii) significantly higher yields of CAR T cells compared to HDR; iv) enrichment of correctly targeted CAR T cells; and v) manufacturing of CAR T cells at therapeutically relevant dose levels.

The methods and compositions combine HITI with CRISPR/Cas9 based gene editing to insert (“knock-in”) a CAR transgene into a specific genomic locus of a T cell.

### General

The practice of the present disclosure employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, Molecular Cloning: A Laboratory Manual, 2nd edition (1989), Current Protocols in Molecular Biology (F. M. Ausubel, et al. eds., (1987), the series Methods in Enzymology (Academic Press. Inc.): PCR 2: A Practical Approach (M. J. MacPherson. B. D. Hames and G. R. Taylor eds. (1995), Antibodies, A Laboratory Manual, and Animal Cell Culture (R. I. Freshney, ed. (1987).

Oligonucleotides that are not commercially available can be chemically synthesized, e.g., according to the solid phase phosphoramidite triester method first described by Beaucage and Caruthers, Tetrahedron Let. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### Terminology

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For purposes of the present disclosure, the following terms are defined.

The terms “a.” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” in relation to a reference numerical value can include a range of values plus or minus 10% from that value. For example, the amount “about 10” includes amounts from 9 to 11. including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

The term “genome editing” refers to a type of genetic engineering in which DNA is inserted, replaced, or removed from a target DNA (e.g., the genome of a cell) using one or more nucleases. The nucleases create specific double-strand breaks (DSBs) at desired locations in the genome, and harness the cell's endogenous mechanisms to repair the induced break by homology-directed repair (HDR) (e.g., homologous recombination) or by nonhomologous end joining (NHEJ).

The terms “genetic modification,” “genetic edit,” and “genome edit” can be used interchangeably and refer to a change in the nucleic acid sequence of a target polynucleotide (e.g., the genomic DNA of a cell), such that the nucleic acid sequence of the modified DNA is different from the native, endogenous, previously modified, or wild-type sequence of the target DNA. The term encompasses mutations and variants of the target DNA sequence, and includes insertions, replacements, or deletions of the target polynucleotide sequence, including insertion of a CAR sequence at a target genomic DNA locus.

The term “DNA nuclease” refers to an enzyme capable of cleaving the phosphodiester bonds between the nucleotide subunits of DNA, and may be an endonuclease or an exonuclease. According to the present disclosure, the DNA nuclease may be an engineered (e.g., programmable or targetable) DNA nuclease which can be used to induce genome editing of a target DNA sequence. Any suitable DNA nuclease can be used including, but not limited to, CRISPR-associated protein (Cas) nucleases, other endo- or exo-nucleases, variants thereof. fragments thereof, and combinations thereof.

The term “double-strand break” or “double-strand cut” refers to the severing or cleavage of both strands of the DNA double helix. The DSB may result in cleavage of both stands at the same position leading to “blunt ends” or staggered cleavage resulting in a region of single-stranded DNA at the end of each DNA fragment, or “sticky ends”. A DSB may arise from the action of one or more DNA nucleases.

The term “nonhomologous end joining” or “NHEJ” refers to a pathway that repairs double-strand DNA breaks in which the break ends are directly ligated without the need for a homologous template.

The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically. degenerate codon substitutions may be achieved by generating sequences in which the third position of one or more selected (or all) codons is substituted with mixed-base and/or deoxyinosine residues (Batzer et al., Nucleic Acid Res. 19:5081 (1991), Ohtsuka et al., J. Biol. Chem. 260:2605-2608 (1985); and Rossolini et al., Mol. Cell. Probes 8:91-98 (1994)).

The term “guide RNA” refers to a nucleic acid molecule comprising an RNA molecule, where the RNA comprises a direct repeat stem loop sequence and a spacer sequence that can bind to a DNA target sequence via RNA-DNA hybridization.

The terms “encode” or encodes” refer to a nucleic acid sequence comprising an open reading frame that can be transcribed by an RNA polymerase and translated into a polypeptide or protein of the disclosure (e.g. a chimeric antigen receptor or selectable marker). The term also includes nucleic acid sequences that are transcribed to produce an RNA that is not translated into a polypeptide or protein, such as a nucleic acid sequence encoding a guide array of the disclosure.

As used herein the term “non-targeting control guide” is a crRNA with a spacer that was randomly generated and BLASTED against the human transcriptome to ensure there were no matches to any known human RNA transcripts.

The term “gene” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The term “cassette” refers to a combination of genetic sequence elements that may be introduced as a single element and may function together to achieve a desired result. A cassette typically comprises polynucleotides in combinations that are not found in nature. A cassette can be inserted into a vector, such as an expression vector.

The term “operably linked” refers to two or more genetic sequence elements, such as a polynucleotide coding sequence and a promoter sequence, placed in relative positions in a polynucleotide, cassette or vector that permit the proper biological functioning of the elements, such as the promoter binding an RNA polymerase that transcribes the coding sequence.

The terms “vector” and “expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators).

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3. John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide).

As used herein, the term “heterologous” refers to biological material that is introduced, inserted, or incorporated into a recipient (e.g., host) organism that originates from another organism. Typically, the heterologous material that is introduced into the recipient organism (e.g., a host cell) is not normally found in that organism. Heterologous material can include, but is not limited to, nucleic acids, amino acids, peptides, proteins, and structural elements such as genes, promoters, and cassettes. A host cell can be, but is not limited to, a bacterium, a yeast cell, a mammalian cell, or a plant cell. The introduction of heterologous material into a host cell or organism can result, in some instances, in the expression of additional heterologous material in or by the host cell or organism. As a non-limiting example, the transformation of a yeast host cell with an expression vector that contains DNA sequences encoding a bacterial protein may result in the expression of the bacterial protein by the yeast cell. The incorporation of heterologous material may be permanent or transient. Also, the expression of heterologous material may be permanent or transient.

The terms “reporter” and “selectable marker” can be used interchangeably and refer to a gene product that permits a cell expressing that gene product to be identified and/or isolated from a mixed population of cells. Such isolation might be achieved through the selective killing of cells not expressing the selectable marker, which may be, as a non-limiting example, an antibiotic resistance gene or a protein conferring resistance to a drug or compound (e.g., DHFR confers resistance to MTX). Alternatively, the selectable marker may permit identification and/or subsequent isolation of cells expressing the marker as a result of the expression of a fluorescent protein such as GFP or the expression of a cell surface marker which permits isolation of cells by fluorescence-activated cell sorting (FACS), magnetic-activated cell sorting (MACS), or analogous methods. Suitable cell surface markers include tEGFR and tNGFR. Suitable markers and techniques are known in the art.

The terms “culture,” “culturing,” “grow,” “growing,” “maintain,” “maintaining,” “expand,” “expanding,” etc., when referring to cell culture itself or the process of culturing, can be used interchangeably to mean that a cell (e.g., yeast cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

The terms “subject.” “individual,” and “patient” are used interchangeably herein to refer to a vertebrate, preferably a mammal, more preferably a human Mammals include, but are not limited to, murines, simians, humans, farm animals, sport animals, and pets. Tissues, cells and their progeny of a biological entity obtained in vivo or cultured in vitro are also encompassed.

As used herein, the term “administering” includes oral administration, topical contact. administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by a medical professional of ordinary skill in the art. The specific amount may vary depending on one or more of: the particular agent chosen, the host cell type, the location of the host cell in the subject, the dosing regimen to be followed, whether it is administered in combination with other compounds, timing of administration, and the physical delivery system in which it is carried.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the patient. Non-limiting examples of pharmaceutically acceptable carrier include water, NaCl, normal saline solutions, lactated Ringer's, normal sucrose, normal glucose, and cell culture media. One of skill in the art will recognize that other pharmaceutical carriers are useful in the present disclosure.

### Methods for Enriching CAR T Cells

In one aspect, the disclosure provides methods for enriching CAR T cells. In some embodiments, the method comprises transfecting a plurality of T cells with
(a) a linear nucleic acid molecule comprising:
(i) an expression cassette comprising a promoter operably linked to a sequence encoding a CAR and a sequence encoding a selectable marker; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i); and
(b) a guide RNA-CRISPR/Cas9 ribonucleoprotein (RNP);
wherein the nucleic acid molecule is inserted into a target site in the genome of the T cell via non-homologous end joining (NHEJ); and isolating T cells that express the selectable marker.

In some embodiments, the number of CAR+ T cells is increased compared to T cells that do not express the selectable marker.

In some embodiments, the CAR transgene is inserted into a target site in the genome of the T cell via NHEJ and not via HDR. Thus, in some embodiments, the nucleic acid molecule comprises sequences encoding a CAR, but does not comprise sequences that are homologous to genomic sequences at the target genomic locus.

In some embodiments, the single PAM is located 5′ of the expression cassette.

In some embodiments, the nucleic acid molecule is inserted on the positive or negative strand of the target genomic locus. In some embodiments, the nucleic acid molecule is inserted on the negative strand of the target genomic locus to avoid potential interference with an endogenous promoter at the target site in the genome.

In some embodiments, the CAR transgene is integrated into a so called “safe-harbor” locus in the genome of the T cell. A safe-harbor locus or safe harbor site is a genomic location where new genes or genetic elements can be introduced without disrupting the expression or regulation of adjacent genes. In some embodiments, the CAR transgene is integrated into the T cell receptor alpha constant (TRAC) genomic locus, the beta-2-microglobulin (B2M) genomic locus, or the adeno-associated virus integration site 1 (AAVS1) genomic locus. Other examples of safe harbor loci include but are not limited to the human CCR5 and hROSA26 genetic loci. Additional safe harbor sites are described in Pellenz S, et al., New Human Chromosomal Sites with “Safe Harbor” Potential for Targeted Transgene Insertion. Hum Gene Ther. 2019 July; 30(7):814-828. doi: 10.1089/hum.2018.169.

In some embodiments, the plurality of T cells comprises primary T cells, such as human primary T cells. In some embodiments, the plurality of T cells comprises unstimulated or unactivated T cells. In some embodiments, the plurality of T cells comprises activated T cells. In some embodiments, the plurality of T cells are activated prior to transfection with the nuclei acid molecule and guide RNA-CRISPR/Cas9 ribonucleoprotein (RNP). In some embodiments, transfection of the T cells comprises electroporation.

### Nucleic Acids, Plasmids and Vectors

In some embodiments, the nucleic acid molecule is a recombinant DNA molecule comprising a plasmid or vector having a relatively small origin of replication, which allows for relatively larger transgene insert sizes. In some embodiments, the nucleic acid molecule comprises a plasmid or vector having an origin of replication comprising less than about 500 bp, e.g., less than about 500, 450, 400, 350, or 300 bp. In some embodiments, the plasmid or vector comprises a sequence encoding a selectable marker, wherein the sequence encoding the selectable marker is less than about 200 bp, e.g., less than about 200, 190, 180, 170, 160, 150, or 140 bp. In some embodiments, the plasmid or vector comprises an origin of replication and sequences encoding a selectable marker, wherein the origin or replication sequence and the sequences encoding the selectable marker combined (e.g., the plasmid backbone without an insert sequence) are less than about 1000 bp, e.g., less than about 1000, 900, 800, 700, 600, or 500 bp. In some embodiments, the plasmid or vector comprises an insert size greater than or equal to 1000 bp, e.g., greater than or equal to 1000, 1500, 2000, 2500, 3000, 3500, 4000, 4500, 5000, 5500, 6000, 6500, 7000, 8000, or 9000 bp, or greater than or equal to 10 kb, 11 kb, 12 kb, 13 kb, 14 kb, 15 kb, 16 kb, 17 kb, 18 kb, 19 kb or 20 kb. In some embodiments, the plasmid comprises an insert size of at least 2000 bp. In some embodiments, the plasmid comprises an insert size between 2000 and 5000 bp. Examples of suitable plasmids and expression vectors are described in US Patent Publication 2021/0010021 A1.

In some embodiments, the nucleic acid molecule comprises a non-viral expression cassette that does not contain any bacterial plasmid DNA sequences, such as a minicircle DNA. Examples of minicircle DNA vectors that can be used to integrate contiguous sequences greater than 10 kb are described in U.S. Pat. No. 9,233,174 B2.

In some embodiments, the nucleic acid molecule comprises an expression cassette comprising a promoter operably linked to a sequence encoding a CAR. In some embodiments, the promoter is an EF-1α promoter or EF-1α short (EFS) promoter. In some embodiments, the EFS promoter comprises the nucleic acid sequence of SEQ ID NO:1. In some embodiments, the EF-1α promoter comprises the nucleic acid sequence of SEQ ID NO:2.

Any of a number of transcription and translation control elements, including promoter, transcription enhancers, and transcription terminators, may be used in the expression vector. Useful promoters can be derived from viruses, or any organism, e.g., prokaryotic or eukaryotic organisms. Promoters may also be inducible (i.e., capable of responding to environmental factors and/or external stimuli that can be artificially controlled) or constitutive. Suitable promoters include, but are not limited to: RNA polymerase II promoters (e.g., EF-1α promoter or EF-1α short (EFS) promoter, pGAL7 and pTEF1), RNA polymerase III promoters (e.g., RPR-tetO, SNR52, and tRNA-tyr), the SV40 early promoter, mouse mammary tumor virus long terminal repeat (LTR) promoter; adenovirus major late promoter (Ad MLP): a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc. Suitable terminators include, but are not limited to SNR52 and RPR terminator sequences, which can be used with transcripts created under the control of a RNA polymerase III promoter. Additionally, various primer binding sites may be incorporated into a vector to facilitate vector cloning, sequencing, and genotyping. Other suitable promoter, enhancer, terminator, and primer binding sequences will readily be known to one of skill in the art.

### Inhibitors of Homology Directed Repair

In some embodiments, the T cells are incubated with an inhibitor of homology directed repair (HDR). In some embodiments, the T cells are incubated with an inhibitor of HDR after transfection with the nuclei acid molecule. In some embodiments, the inhibitor of HDR is selected from the group consisting of an ATM/ATR kinase inhibitor, a Chk1/Chk2 inhibitor, a BRCA1 inhibitor, a Rad51 inhibitor, and combinations thereof. In some embodiments, the ATM/ATR kinase inhibitors is selected from the group consisting of AZD0156, Ceralasertib (AZD6738), AZ31, VX-803, AZD1390, AZ32, M4076, and AZ20. In some embodiments, the Chk1/Chk2 inhibitor is selected from the group consisting of Rabusertib (LY2603618), BML-277, CHIR-124, PF-477736, MK-8776, SAR-020106, PD0166285, Prexasertib (LY2606368), and AZD7762. In some embodiments, the BRCA1 inhibitor is selected from BRCA1-IN-1 or Bractoppin. In some embodiments, the Rad51 inhibitor is selected from the group consisting of B02, B02-iso (or an analog thereof), RI-1, and CAM833. Additional Rad51 inhibitors are available from MCE® medchemexpress.

### Selectable Markers

In some embodiments, the nucleic acid molecule comprises an expression cassette comprising a promoter operably linked to a sequence encoding a selectable marker. The selectable marker can be used to enrich (i.e., increase the frequency) T cells that integrate the sequence encoding the CAR into the genome. The selectable marker can be a protein expressed on the cell surface of the CAR T cell, or an intracellular protein expressed by the CAR T cell.

In some embodiments, the selectable marker is a protein expressed on the cell surface of the CART cell, and expression of the selectable marker is detected by contacting an antibody to the selectable marker. In some embodiments, the protein expressed on the cell surface of the CAR T cell is tEGFR or tNGFR. In some embodiments, the antibody used to detect tEGFR is the clinically approved antibody Cetuximab. In some embodiments, the antibody used to detect tEGFR is AY13 (PE/Biotin, Biolegend cat. no. #352904/3352934). In some embodiments, the antibody used to detect tNGFR is ME20.4 (PE/Biotin, Biolegend cat. no. #345106/#345122). When using a cell surface selectable marker such as tEGFR or tNGFR, enrichment can be determined by column separation such as LS-columns. In some embodiments, the antibody that binds to the selectable marker can be conjugated to biotin and detected by binding to streptavidin, for example by using the QuadroMACS magnetic column separation system along with Streptavidin Microbeads (Miltenyi Biotec) or using the EasySep Biotin Positive Selection kit for column-free separation (STEMCELL Technologies). In some embodiments, the enrichment using a cell surface selectable marker is determined 14 days after T cell activation.

In some embodiments, the selectable marker is an intracellular protein expressed by the CAR T cell. In some embodiments, the selectable marker is an intracellular protein that confers resistance to a drug or compound. In some embodiments, the selectable marker is a dihydrofolate reductase (DHFR), an intracellular protein. In some embodiments, the DHFR comprises two amino acid substitutions, L22F and F31S, in the amino acid sequence (referred to as “DHFR-FS”). DHFR-FS confers resistance against the FDA approved drug methotrexate (MTX). Thus, in some embodiments, the selected marker is DHFR-FS and the drug or compound is MTX. In some embodiments, the T cells are cultured in the presence of MTX for a period of time to reduce the number of cells that do not express DHFR-FS.

In some embodiments, the T cells are activated prior to culturing with MTX. In some embodiments, the T cells are cultured with MTX beginning on day 3 up to day 7 post-activation, where the T cells are activated on day 0 (e.g., cultured with MTX beginning on day 3, 4, 5, 6, or 7 post-activation). In some embodiments, the T cells are transfected with the nucleic acid molecule on day 2 after activation, and cultured with MTX beginning on days 1 to 5 post-transfection (e.g., cultured with MTX beginning on day 1, 2, 3, 4, or 5, post-transfection). In some embodiments, the T cells are cultured with MTX for 1, 2, 3, 4, 5, 6 or 7 days. In some embodiments, the T cells are cultured with MTX to reduce the number of cells that do not express the CAR (CAR negative T cells), and are then cultured without MTX for at least 1 day to determine the percentage of viable (living) cells. In some embodiments, the T cells are cultured with MTX to reduce the number of cells that do not express the CAR (CAR negative T cells), and are then cultured without MTX for 1 to 7 days, e.g., cultured without MTX for 1, 2, 3, 4, 5, 6, or 7 days. It will be understood that the duration the T cells are cultured without MTX can varying depending on the desired outcomes, considering the balance between T cell yield and T cell exhaustion due to extended ex vivo culture.

In some embodiments, the T cells are cultured with the drug or compound to increase the frequency of CAR+ T cells prior to administering the CAR T cells to a patient. In some embodiments, the T cells express the selectable marker DHFR-FS and the T cells are cultured in the presence of MTX for 1 to up to 7 days (e.g., cultured with MTX for 1, 2, 3, 4, 5, 6 or 7 days), and then cultured without MTX for at least 1 day (e.g., for 1, 2, 3, 4, 5, 6, or 7 days). Following the culture period without MTX, the number of viable (living) CAR T cells is determined prior at administering the T cells to a patient. In some embodiments, the number of viable T cells is greater than or equal to 70% prior to administering the T cells to a patient.

In some embodiments, the T cells are cultured with the drug or compound beginning earlier after T cell activation than standard protocols to decrease the amount of time that the T cells are cultured in vitro, which can improve clinical outcomes. For example, previous protocols began treatment with MTX one week after T cell activation and continued MTX treatment until the end of the culture period at 14 days post-activation (ref. 40). The instant disclosure provides a shortened, optimized time period of culture in MTX, beginning on day 3 after T cell activation (day 1 after transfection) for a duration of 4 days, ending on day 7 after T cell activation (day 5 after transfection). In some embodiments, the T cells are then cultured without MTX for an additional 7 days (day 14 after activation) before determining the frequency of T cells that express expression the CAR. The optimized protocol produced CAR T cell frequencies equal to or greater than 70%.

In some embodiments, the T cells express the selectable marker DHFR-FS and the T cells are cultured in the presence of MTX beginning on day 3 to day 7 after activation (day 1 after transfection until day 5 after transfection), and are then cultured without MTX until day 14 after activation (day 12 after transfection), and the yield of CAR+ T cells at day 14 is similar to the yield of CAR+ T cells cultured with MTX beginning on day 7 after activation until day 14 after activation.

In some embodiments, the T cells express the selectable marker DHFR-FS and the T cells are cultured in the presence of MTX beginning on day 3 to day 7 after activation (day 1 after transfection until day 5 after transfection), and are then cultured without MTX until day 10 after activation (day 8 after transfection).

In some embodiments the T cells are activated with Dynabeads™ coated with anti-CD3 and anti-CD28 antibodies (Thermo Fisher). In some embodiments the T cells are activated with human T Cell Transact™ via CD3 and CD28 (Miltenyi Biotec B.V. & Co.).

In some embodiments, the T cells are enriched using the tEGFR or tNGFR selectable markers. In some embodiments, the enrichment for tEGFR and tNGFR happens as a single step at one time point. In some embodiments, the enrichment timepoint occurs near the middle of the culture period, e.g., at day 9 after activation. In some embodiments, the enrichment timepoint occurs near the end of the culture period, e.g., at day 14 after activation.

### CAR-T Cells

In one aspect, the T cells of the disclosure comprise a nucleic acid sequence encoding a CAR that is expressed on the cell surface of the CAR T cell. CAR T cells express a receptor that binds an antigen on a target cell, such as a tumor cell, and activates native T cell functionality to target and kill the target cell. First-generation CAR T cell therapeutic receptors comprise an extracellular antigen binding domain and an intracellular T cell activating domain. Second-generation CAR T cell therapeutic receptors include both a costimulatory domain and a T cell activation domain on their intracellular side. The costimulatory domain improves the therapeutic response of T cells by increasing T cell proliferation or cytotoxicity. In some embodiments, the CAR comprises an antigen binding scFv or nanobody. In some embodiments, the CAR comprises a CD8 transmembrane domain. In some embodiments, the CAR comprises the intramembrane signaling domains CD28 and CD3ζ (CD247). In some embodiments, the CAR comprises the intramembrane signaling domains 4-1BB (CD137) and CD3ζ. In some embodiments, the CAR comprises a P2A self-cleaving peptide. In some embodiments, the CAR plasmid construct comprises an EF-1a short (EFS) promoter and a signal peptide upstream (Y) of the CAR coding sequence. In some embodiments, the CAR plasmid construct comprises a DHFR-FS selectable marker located downstream (3′) of the CAR coding sequence.

In some embodiments, the CAR binds to an antigen expressed by a tumor. In some embodiments, the CAR binds to an antigen selected from the group consisting of Her-2, B7-H3, GPC2, GD2. CD19, CD20, CD22, MAGE, BAGE, CAGE, GAGE, HAGE, LAGE. PAGE, PRAME, NY-ESO-1, NY-SEO-1, tyrosinase, Melan-A/MART, gp100, TRP-1, TRP-2, CD30, EGFR. EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA. AFP, BCMA, Mesothelin, GPC3, MUC1 and CTAG1B. In some embodiments, the CAR binds to a GD2 antigen.

### CRISPR/Cas System

The CRISPR/Cas system of genome modification includes a Cas nuclease (e.g., Cas9 or Cpf1 nuclease) or a variant or fragment or combination thereof and a DNA-targeting RNA (e.g., guide RNA (gRNA)). The gRNA may contain a guide sequence that targets the Cas nuclease to the target genomic DNA and a scaffold sequence that interacts with the Cas nuclease (e.g., tracrRNA). The system may optionally include a donor repair template. In other instances, a fragment of a Cas nuclease or a variant thereof with desired properties (e.g., capable of generating single- or double-strand breaks and/or modulating gene expression) can be used. The donor repair template can include a nucleotide sequence encoding a reporter polypeptide such as a fluorescent protein or an antibiotic resistance marker, and homology arms that are homologous to the target DNA and flank the site of gene modification.

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called tracrRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease may require both the crRNA and the tracrRNA for site-specific DNA recognition and cleavage. This system has now been engineered such that the crRNA and tracrRNA, if needed, can be combined into one molecule (the “single guide RNA” or “sgRNA”), and the crRNA equivalent portion of the guide RNA can be engineered to guide the Cas (e.g., Cas9) nuclease to target any desired sequence (see, e.g., Jinek et al. (2012) Science. 337:816-821; Jinek et al. (2013) eLife, 2:e00471; Segal (2013) eLife, 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

The Cas nuclease can direct cleavage of one or both strands at a location in a target DNA sequence.

Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Csc1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, fragments thereof, mutants thereof, derivatives thereof, and combinations thereof. There are three main types of Cas nucleases (type I, type II, and type III), and 10 subtypes including 5 type 1, 3 type II, and 2 type III proteins (see, e.g., Hochstrasser and Doudna, Trends Biochem Sci, 2015:40(1):58-66). Type II Cas nucleases include Cas1, Cas2, Csn2, Cas9, and Cpf1. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. Furthermore, the amino acid sequence of Acidaminococcus sp. BV3L6 is set forth, e.g., in NBCI Ref. Seq. No. WP_021736722.1. Some CRISPR-related endonucleases that are useful in the present disclosure are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifldobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradirhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cpf1” refers to an RNA-guided double-stranded DNA-binding nuclease protein that is a type II Cas nuclease. Wild-type Cpf1 contains a RuvC-like endonuclease domain similar to the RuvC domain of Cas9, but does not have an HNH endonuclease domain and the N-terminal region of Cpf1 does not have the alpha-helix recognition lobe possessed by Cas9. The wild-type protein requires a single RNA molecule, as no tracrRNA is necessary. Wild-type Cpf1 creates staggered-end cuts and utilizes a T-rich protospacer-adjacent motif (PAM) that is 5′ of the guide RNA targeting sequence. Cpf1 enzymes have been isolated, for example, from Acidaminococcus and Lachnospiraceae.

“Cas9” refers to an RNA-guided double-stranded DNA-binding nuclease protein or nickase protein that is a type 11 Cas nuclease. Wild-type Cas9 nuclease has two functional domains, e.g., RuvC and HNH, that cut different DNA strands. The wild-type enzyme requires two RNA molecules (e.g., a crRNA and a tracrRNA), or alternatively, a single fusion molecule (e.g., a gRNA comprising a crRNA and a tracrRNA). Wild-type Cas9 utilizes a G-rich protospacer-adjacent motif (PAM) that is 3′ of the guide RNA targeting sequence and creates double-strand cuts having blunt ends. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Fubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

Useful variants of the Cas9 nuclease can include a single inactive catalytic domain, such as a RuvC⁻ or HNH⁻ enzyme. The Cas9 nuclease can be codon-optimized for the host cell or host organism.

For genome editing methods, the Cas nuclease can be a Cas9 fusion protein such as a polypeptide comprising the catalytic domain of a restriction enzyme (e.g., FokI) linked to dCas9. The FokI-dCas9 fusion protein (fCas9) can use two guide RNAs to bind to a single strand of target DNA to generate a double-strand break.

In some embodiments, a nucleotide sequence encoding the Cas nuclease is present in a recombinant nucleic acid molecule described above. Useful expression vectors containing a nucleotide sequence encoding a Cas9 enzyme are also commercially available from, e.g., Addgene, Life Technologies, Sigma-Aldrich, and Origene.

Guide RNA (gRNA) Molecules

The gRNAs for use in the CRISPR/Cas system of the present disclosure typically include a crRNA sequence that is complementary to a target nucleic acid sequence and may include a scaffold sequence (e.g., tracrRNA) that interacts with a Cas nuclease (e.g., Cas9) or a variant or fragment thereof, depending on the particular nuclease being used. In some embodiments, the gRNA binds to a sequence in a “safe harbor” locus in the genome of the T cell, as described herein. In some embodiments, the gRNA binds to a sequence in the T cell receptor alpha constant (TRAC) genomic locus, the beta-2-microglobulin (B2M) genomic locus, or the adeno-associated virus integration site 1 (AAVS1) genomic locus.

The gRNA can comprise any nucleic acid sequence having sufficient complementarity with a target polynucleotide sequence (e.g., target DNA sequence) to hybridize with the target sequence and direct sequence-specific binding of a nuclease to the target sequence. The gRNA may recognize a protospacer adjacent motif (PAM) sequence that may be near or adjacent to the target DNA sequence. The target DNA site may lie immediately 5′ of a PAM sequence, which is specific to the bacterial species of the Cas9 used. For instance, the PAM sequence of Streptococcus pyogenes-derived Cas9 is NGG; the PAM sequence of Neisseria meningitidis-derived Cas9 is NNNNGATT; the PAM sequence of Streptococcus thermophilus-derived Cas9 is NNAGAA; and the PAM sequence of Treponema denticola-derived Cas9 is NAAAAC. In some embodiments, the PAM sequence can be 5′-NGG, wherein N is any nucleotide; 5′-NRG, wherein N is any nucleotide and R is a purine; or 5′-NNGRR, wherein N is any nucleotide and R is a purine. For the S. pyogenes system. the selected target DNA sequence should immediately precede (i.e., be located 5′ of) a 5′NGG PAM. wherein N is any nucleotide, such that the guide sequence of the DNA-targeting RNA (e.g., gRNA) base pairs with the opposite strand to mediate cleavage at about 3 base pairs upstream of the PAM sequence.

In other instances, the target DNA site may lie immediately 3′ of a PAM sequence, e.g., when the Cpf1 endonuclease is used. In some embodiments, the PAM sequence is 5′-TTTN, where N is any nucleotide. When using the Cpf1 endonuclease, the target DNA sequence (i.e., the genomic DNA sequence having complementarity for the gRNA) will typically follow (i.e., be located 3′ of) the PAM sequence. Two CP1-family nucleases, AsCpf1 (from Acidaminococcus) and LbCpf1 (from Lachnospiraceae) are known to function in human cells. Both AsCpf1 and LbCpf1 cut 19 bp after the PAM sequence on the targeted strand and 23 bp after the PAM sequence on the opposite strand of the DNA molecule.

In some embodiments, the degree of complementarity between a guide sequence of the gRNA (i.e., crRNA sequence) and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner). ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a crRNA sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some instances, a crRNA sequence is about 20 nucleotides in length. In other instances, a crRNA sequence is about 15 nucleotides in length. In other instances, a crRNA sequence is about 25 nucleotides in length.

The nucleotide sequence of a modified gRNA can be selected using any of the web-based software described above. Considerations for selecting a DNA-targeting RNA include the PAM sequence for the nuclease (e.g., Cas9 or Cpf1) to be used, and strategies for minimizing off-target modifications. Tools, such as the CRISPR Design Tool, can provide sequences for preparing the gRNA, for assessing target modification efficiency, and/or assessing cleavage at off-target sites.

In some embodiments, the length of the gRNA molecule is about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more nucleotides in length. In some instances, the length of the gRNA is about 100 nucleotides in length. In other instances, the gRNA is about 90 nucleotides in length. In other instances, the gRNA is about 110 nucleotides in length.

### Genetically Modified T Cells

In another aspect, the disclosure provides a genetically modified T cell or a plurality of genetically modified T cells. In some embodiments, the genetically modified T cell(s) comprises a nucleic acid molecule comprising:
(i) an expression cassette comprising a promoter operably linked to a sequence encoding a CAR and a sequence encoding a selectable marker; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i).

In some embodiments, the promoter is an EF-1α promoter or EF-1α short (EFS) promoter. In some embodiments, the EFS promoter comprises the nucleic acid sequence of SEQ ID NO:1. In some embodiments, the EF-1α promoter comprises the nucleic acid sequence of SEQ ID NO:2.

In some embodiments, the single PAM is located 5′ of the expression cassette.

In some embodiments, the CAR binds to an antigen selected from the group consisting of Her-2. B7-H3, GPC2, GD2. CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4. CSPG4, B7-H4, NKG2D, CS1. CD138, EpCAM. EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

In some embodiments, the selectable marker is a protein expressed on the cell surface of the genetically modified T cell(s), or an intracellular protein expressed by the genetically modified T cell(s).

In some embodiments, the selectable marker is an intracellular protein that confers resistance to a drug or compound. In some embodiments, the selectable marker is a dihydrofolate reductase (DHFR), an intracellular protein. In some embodiments, the DHFR comprises two amino acid substitutions, L22F and F31S, in the amino acid sequence (referred to as “DHFR-FS”).

In some embodiments, the selectable marker is a protein expressed on the cell surface of the genetically modified T cell(s). In some embodiments, the protein expressed on the cell surface of the genetically modified T cell(s) is tEGFR or tNGFR.

In some embodiments, the nucleic acid molecule is inserted on the positive or negative strand of the target genomic locus. In some embodiments, the nucleic acid molecule is inserted on the negative strand of the target genomic locus to avoid potential interference with an endogenous promoter at the target site in the genome.

In some embodiments, the nucleic acid molecule is integrated into a so called “safe-harbor” locus in the genome of the T cell, as described herein. In some embodiments, the nucleic acid molecule is integrated into the T cell receptor alpha constant (TRAC) genomic locus, the beta-2-microglobulin (B2M) genomic locus, or the adeno-associated virus integration site 1 (AAVS1) genomic locus.

In some embodiments, the genetically modified T cell(s) is a primary human T cell. In some embodiments, the genetically modified T cell(s) is an activated T cell. In some embodiments, the genetically modified T cell(s) is an unstimulated T cell.

In some embodiments, the genetically modified T cell(s) further comprises a guide RNA and CRISPR/Cas9 nuclease ribonucleoprotein (RNP).

### Pharmaceutical Compositions

In another aspect, the disclosure provides a pharmaceutical composition comprising a plurality of genetically modified T cell(s) described herein as an active ingredient. In some embodiments, the pharmaceutical composition is formulated for delivery to a subject or patient. Representative formulations for genetically modified T cell(s), including CAR T cells, include cryogenically preserved T cells. In some embodiments, the pharmaceutical composition is provided in a cryogenic infusion bag or cryogenic vial.

The pharmaceutical compositions of the present disclosure, including cells and/or progeny thereof that have had their genomes edited by the methods and/or compositions of the present disclosure, can be administered as a single dose or as multiple doses, for example two doses administered at an interval of about one month, about two months, about three months, about six months or about 12 months. The dose of the pharmaceutical compositions can be administered to the subject in a single dose, or split doses over several days. Other suitable dosage schedules can be determined by a medical practitioner.

In some embodiments, the dose of the active ingredient (e.g., CAR T cells) administered to the subject comprises about 1×10⁶ to 1×10⁹ viable CAR+ T cells (e.g., about 1×10⁶, 5×10⁶, 1×10⁷, 5×10⁷, 1×10⁸, 5×10⁸, 1×10⁹, or 5×10⁹ viable CAR+ T cells). In some embodiments, the dose of the active ingredient (e.g., CAR T cells) administered to the subject comprises about 0.1×10⁶ to 5×10⁶ viable CAR+ T cells/kg of the subject's weight. Representative doses of marketed T cells products include 2×10⁶ viable CAR T cells/kg body weight, with a maximum dose of 2×10⁸ viable CAR T cells for brexucabtagene autoleucel (Tecartus™) and axicabtagene ciloleucel (Yescarta®), 0.6-6.0×10⁸ viable CAR T cells for Tisagenlecleucel (Kymriah®), and 3.0-4.6×10⁸ viable CART cells for idecabtagene vicleucel (Abecma®). See van der Walle C F, et al., Formulation Considerations for Autologous T Cell Drug Products. Pharmaceutics. 2021 Aug. 23:13(8):1317. doi: 10.3390/pharmaceutics13081317. Other suitable dosages of active ingredients comprising genetically modified CAR T cells of the disclosure can be determined by a medical practitioner.

The pharmaceutical compositions and formulations can be combined with a pharmaceutically acceptable carrier or excipient for administration to a subject or patient. In some embodiments, the pharmaceutically acceptable carrier comprises a carrier or excipient that can be included in the compositions of the disclosure and that causes no significant adverse toxicological effect on the patient. Non-limiting examples of pharmaceutically acceptable carrier include water. NaCl. normal saline solutions, lactated Ringer's, normal sucrose, normal glucose, human serum albumin (HSA), DMSO, and cell culture media. In some embodiments, the excipient comprises 2.5% (+/−25%) w/v HSA and 5% (+/−20%) w/v DMSO (see See van der Walle C F, et al., Formulation Considerations for Autologous T Cell Drug Products. Pharmaceutics. 2021 Aug. 23; 13(8):1317. doi: 10.3390/pharmaceutics13081317). One of skill in the art will recognize that other pharmaceutical carriers are useful in the present disclosure. Suitable pharmaceutical carriers are known in the art, and are described, for example, in the ASHP Handbook on Injectable Drugs, Trissel, 18th ed. (2014) and Handbook of Phannaceutical Excipients Ninth edition, Paul J Sheskey, Bruno C Hancock, Gary P Moss, David J Goldfarb, eds, (2020).

The pharmaceutical compositions can be delivered to a subject via any medically acceptable route, including local or systemic administration. For example, in some embodiments, the pharmaceutical composition is administered by injection, such as intravenous, subcutaneous, intramuscular, or intraperitoneal administration.

Prevention or treatment can further comprise administering agents and/or performing procedures to prevent or treat concomitant or related conditions. As non-limiting examples, it may be necessary to administer drugs to suppress immune rejection of transplanted cells, or prevent or reduce inflammation or infection. A medical professional will readily be able to determine the appropriate concomitant therapies.

### Methods of Treatment

Also provided are methods for treating a cancer or tumor in a subject. In some embodiments, the method comprises administering to a subject an amount of a pharmaceutical composition of the disclosure to the subject that is effective to treat the cancer or tumor. In some embodiments, the method comprises administering a plurality of genetically modified T cells of the disclosure to a subject in an amount effective to treat the cancer or tumor. In some embodiments, the method comprises administering a therapeutic amount of genetically modified T cells to the subject, the genetically modified T cells comprising:
a nucleic acid molecule comprising:
(i) an expression cassette comprising a promoter operably linked to a sequence encoding a chimeric antigen receptor (CAR) and a sequence encoding a selectable marker; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i);
wherein the nucleic acid molecule is inserted into a target site in the genome of the T cell via non-homologous end joining (NHEJ), thereby treating the cancer or tumor.

In some embodiments, the plurality of genetically modified T cells that are administered to the subject express the same CAR, e.g., an anti-GD2 CAR.

In some embodiments, the nucleic acid molecule does not comprise sequences that are homologous to genomic sequences at the target genomic locus.

In some embodiments, the single PAM is located 5′ of the expression cassette of (i).

In some embodiments, the nucleic acid molecule comprises a plasmid having a plasmid backbone of less than 500 bp. In some embodiments, the plasmid comprises an insert size between 2000 and 5000 bp.

In some embodiments, the promoter is an EF-1α promoter or EF-1α short (EFS) promoter. In some embodiments, the EFS promoter comprises the nucleic acid sequence of SEQ ID NO:1. In some embodiments, the EF-1α promoter comprises the nucleic acid sequence of SEQ ID NO:2.

In some embodiments, the selectable marker is used to enrich (i.e., increase the frequency) for T cells that integrate and express the sequence encoding the CAR into the genome prior to administering the genetically modified T cells to the subject. In some embodiments, the selectable marker is DHFR-FS, and the T cells are cultured with methotrexate (MTX) for a period of time to reduce the number of cells that do not express DHFR-FS.

In some embodiments, the T cells are activated prior to culturing with MTX. In some embodiments, the T cells are cultured with MTX beginning on days 3 to 7 post-activation, where the T cells are activated on day 0 (e.g., cultured with MTX beginning on day 3, 4, 5, 6, or 7 post-activation). In some embodiments, the T cells are transfected with the nucleic acid molecule on day 2 after activation, and cultured with MTX beginning on days 1 to 5 post-transfection (e.g., cultured with MTX beginning on day 1, 2, 3, 4, or 5, post-transfection). In some embodiments, the T cells are cultured with MTX for 1, 2, 3, 4, 5, 6 or 7 days. In some embodiments, the T cells are cultured with MTX to reduce the number of cells that do not express the CAR (CAR negative T cells), and are then cultured without MTX for at least 1 day to determine the percentage of viable (living) cells, prior to administration to the subject. In some embodiments, the T cells are cultured with MTX to reduce the number of cells that do not express the CAR (CAR negative T cells), and are then cultured without MTX for 1 to 7 days, e.g., cultured without MTX for 1, 2, 3, 4, 5, 6, or 7 days, prior to administration to the subject.

In some embodiments, the T cells express the selectable marker DHFR-FS and the T cells are cultured in the presence of MTX for 1 to up to 7 days (e.g., cultured with MTX for 1, 2, 3, 4, 5, 6 or 7 days), and then cultured without MTX for at least 1 day (e.g., for 1, 2, 3, 4, 5, 6, or 7 days). Following the culture period without MTX, the number of viable (living) CAR T cells is determined prior at administering the T cells to a patient. In some embodiments, the number of viable T cells is greater than or equal to 70%, greater than or equal to 75%, or greater than or equal to 80%, prior to administering the T cells to a subject.

In some embodiments, the T cells are cultured in the presence of MTX, beginning on day 3 after T cell activation (day 1 after transfection) for a duration of 4 days, ending on day 7 after T cell activation (day 5 after transfection). In some embodiments, the T cells are then cultured without MTX for an additional 7 days (day 14 after activation), and the frequency of T cells that express the CAR is determined before administering the T cells to the subject.

In some embodiments, the T cells express the selectable marker DHFR-FS and the T cells are cultured in the presence of MTX beginning on day 3 to day 7 after activation (day 1 after transfection until day 5 after transfection), and are then cultured without MTX until day 14 after activation (day 12 after transfection), prior to administering the T cells to a subject.

In some embodiments, the T cells express the selectable marker DHFR-FS and the T cells are cultured in the presence of MTX beginning on day 3 until day 7 after activation (day 1 after transfection until day 5 after transfection), and are then cultured without MTX until day 10 after activation (day 8 after transfection) prior to administering the T cells to a subject.

In some embodiments the T cells are activated with Dynabeads™ coated with anti-CD3 and anti-CD28 antibodies (Thermo Fisher). In some embodiments the T cells are activated with human T Cell Transact™ via CD3 and CD28 (Miltenyi Biotec B.V. & Co.).

In some embodiments, the selectable marker is a protein expressed on the cell surface. In some embodiments, the selectable marker protein expressed on the cell surface is tEGFR or tNGFR.

In some embodiments, the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

In some embodiments, the nucleic acid molecule is integrated into the T cell receptor alpha constant (TRAC), beta-2-microglobulin (B2M), or adeno-associated virus integration site 1 (AAVS1) genomic locus.

In some embodiments, the T cells are activated prior to transfection with the nucleic acid cassette.

In some embodiments. 1×10⁶ to 1×10⁹ CAR+ T cells (e.g., about 1×10⁶, 5×10⁶, 1×10⁷, 5×10⁷, 1×10⁸, 5×10⁸, 1×10⁹, or 5×10⁹ viable CAR+ T cells) are administered to the subject. In some embodiments, 0.1×10⁶ to 5×10⁶ CAR+ T cells/kg of the subject's weight are administered to the subject.

In some embodiments, the CAR+ T cells are administered to the subject in one or more doses, as described above.

In some embodiments, the cancer is selected from a leukemia, a lymphoma, a melanoma, a neuroendocrine tumor, a carcinoma, or a sarcoma. In some embodiments, the cancer is a colorectal carcinoma, prostate cancer, glioblastoma, or gastric cancer.

In some embodiments, the cancer or tumor can be selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T cell leukemia, Aggressive NK-cell leukemia, AIDS-Related Cancers, AIDS-related lymphoma, Alveolar soft part sarcoma, Ameloblastic fibroma. Anal cancer, Anaplastic large cell lymphoma, Anaplastic thyroid cancer, Angioimmunoblastic T cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia. Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T cell lymphoma, Ependymoblastoma, Ependymoma, Epithelioid sarcoma, Erythroleukemia. Esophageal cancer, Esthesioneuroblastoma. Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma. Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma. Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma. Lymphoepithelioma, Lymphoid leukemia, Lymphoma, Macroglobulinemia. Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology. Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma. Precursor T-lymphoblastic lymphoma, Primary central nervous system lymphoma, Primary effusion lymphoma, Primary Hepatocellular Cancer. Primary Liver Cancer. Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma. Secondary neoplasm. Seminoma, Serous tumor, Sertoli-Leydig cell tumor, Sex cord-stromal tumor, Sezary Syndrome, Signet ring cell carcinoma, Skin Cancer, Small blue round cell tumor. Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T cell acute lymphoblastic leukemia, T cell large granular lymphocyte leukemia. T cell leukemia, T cell lymphoma, T cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma. Visual Pathway Glioma, Vulvar Cancer, Waldenstrom's macroglobulinemia, Warthin's tumor, Wilms' tumor, and combinations thereof.

In some embodiments, the CAR binds an antigen expressed by a cancer or tumor cell selected from the group consisting of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor. Adrenocortical carcinoma, Adult T cell leukemia, Aggressive NK-cell leukemia, AIDS-Related Cancers, AIDS-related lymphoma, Alveolar soft part sarcoma. Ameloblastic fibroma, Anal cancer, Anaplastic large cell lymphoma, Anaplastic thyroid cancer, Angioimmunoblastic T cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma. Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site. Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma. Endodermal sinus tumor, Endometrial cancer, Endometrial Uterine Cancer, Endometrioid tumor, Enteropathy-associated T cell lymphoma, Ependymoblastoma, Ependymoma, Epithelioid sarcoma, Erythroleukemia, Esophageal cancer, Esthesioneuroblastoma. Ewing Family of Tumor. Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer. Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma. Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma. Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides. Mycosis fungoides, Myelodysplastic Disease, Myelodysplastic Syndromes, Myeloid leukemia. Myeloid sarcoma, Myeloproliferative Disease, Myxoma, Nasal Cavity Cancer, Nasopharyngeal Cancer, Nasopharyngeal carcinoma. Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T cell acute lymphoblastic leukemia, T cell large granular lymphocyte leukemia, T cell leukemia, T cell lymphoma, T cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof.

### Kits

In another aspect, the present disclosure provides kit for modifying one or more target nucleic acids of interest at one or more target loci within a genome of a host cell, such as a primary human T cell. In some embodiments, the kit comprises a nucleic acid molecule of the disclosure.

In some embodiments, the kit contains one or more reagents. In some instances, the reagents are useful for transforming a host cell with a nucleic acid molecule described herein. In some embodiments the kit comprises and antibody for detecting a cell surface marker described herein, or a drug or compound useful for selecting genetically modified T cells described herein (e.g., MTX). In some embodiments, the kit further comprises instructions for transforming or electroporating the host cell with a nucleic acid molecule, and/or instructions for selecting or enriching for genetically modified T cells described herein.

### Applications

The compositions and methods provided by the present disclosure are useful for any number of applications. As non-limiting examples, target genome editing or screening of genetically modified T cells according to the compositions and methods of the present disclosure can be used for increasing the yield of CAR T cells, increasing the size of transgenes integrated into the T cell genome, post-editing enrichment of CAR T cells using GMP grade reagents resulting in a more pure product with at least 80% CAR+ cells, and HITI mediated transgene insertion allows manufacturing of CRISPR knock-in CAR T cells at therapeutically relevant dose levels in the range of 5.5×10⁸-3.6×10⁹ CAR+ T cells.

### EXAMPLES

The following Example(s) describe representative, non-limiting methods of the disclosure.

### Example 1

This example describes methods for producing genetically modified T cells comprising a therapeutically relevant CAR transgene using HITI mediated site directed integration into the T cell receptor alpha constant (TRAC) genomic locus.

CAR-T cells are a novel drug class with impressive efficacy in refractory B-cell and plasma cell malignancies¹⁻³. This success is fueling efforts to extend their efficacy to earlier lines of therapy⁴ and for treatment of solid tumors⁵⁻⁹. All current commercially available CAR-T cells use viral vector based transgene delivery¹⁰. Access to these therapies is inadequate to meet clinical need, in part due to high costs and supply chain limitations related to the manufacturing and qualifying GMP grade vectors. Innovation in early stage trials is also limited by long lead times and high costs for production of viral vectors¹¹. Non-viral gene delivery¹²⁻¹⁷ could diminish the cost and complexity of cell manufacturing for CAR T cell therapies. CRISPR/Cas9 with adeno-associated virus 6 (AAV6) can deliver site-directed modifications of the genome via HDR, and this approach may reduce the risk of insertional mutagenesis compared to random insertions delivered via retroviruses and simultaneously knockout a gene of interest^{18,19}. In search of a viral free CAR-T cell manufacturing platform. investigators electroporated CRISPR/Cas9 Ribonucleoprotein (RNP) with linearized or plasmid DNA for targeted transgene knock-in into T cells, but this has been associated with low efficiency and yield due to impaired T cell viability and expansion post editing, which has shown to impact clinical scale manufacturing^{20,21}. More recently, truncated Cas9 target sequences (tCTS) added to ssDNA have enabled efficient knock-in of an anti-BCMA CAR without impairing cell yields²³. However, access to ssDNA for large clinical trials and manufacturing post approval is limited. Additional fully non-viral CRISPR knock-in platforms are needed to ensure adequate access to meet the increasing demand for engineered T cells for therapeutic use.

HDR relies on cell division to provide a sister-chromatid chromosome copy created during S phase, which serves as the template for gene correction after a dsDNA break of the respective locus²². Because HDR is not the predominant DNA repair pathway utilized following dsDNA breaks, NHEJ mediated HITI has more recently been explored for large transgene insertions in both dividing and resting cells²³. Non-homologous end joining (NHEJ) is the primary DNA repair pathway utilized following double-stranded DNA (dsDNA) breaks and acts independent of the cell cycle state. DNA ends are ligated by Ligase IV after Ku proteins recruit nucleases to trim and polymerases to fill in gaps respectively²⁴. HITI resulted in more efficient targeted knock-in when compared to HDR in adherent cell lines and embryonic stem cells, and the increased efficiency was more pronounced when using transgenes of large size (>5 kb)^{25,26}. In vivo gene correction via HITI has been applied pre-clinically for treatment of retinitis pigmentosa. Mucopolysaccharidosis type VI and Adrenoleukodystrophy using either AAV6 or AAV9^{23,27,28}. Due to its cell cycle independent integration, HITI could expand the CRISPR knock-in toolbox for somatic cell and gene therapy²⁹, but it has not been explored for CRISPR knock-in into T cells.

Here, we tested HITI mediated site directed integration of a therapeutically relevant GD2-CAR transgene into the T cell receptor alpha constant (TRAC) locus using plasmid DNA and CRISPR/Cas9 in primary human T cells. Compared to HDR mediated knock-in, HITI yielded at least 2-fold more GD2-CAR-T cells. When combined with CEMENT, HITI GD2-knock-in CAR-T cells were enriched 2.3-8 fold using dihydrofolate reductase^{L22F/F31S} (DHFR-FS), which confers resistance to the FDA approved drug methotrexate (MTX). Using a starting population of 5×10⁸ T cells and a 14-day process, HITI/CEMENT generated GD2-CAR-T cell numbers across 3 independent donors ranging from 5.5×10⁸-3.6×10⁹, sufficient to meet doses administered in all current commercial CAR products. HITI/CEMENT GD2 knock-in CAR-T cells showed an acceptable safety profile as assessed using ddPCR based copy number analysis, unbiased evaluation of off-target sites³² and genome-wide insertion site analysis³³, were functionally equivalent to viral transduced GD2-CAR-T cells and mediated tumor control of an in vivo model of metastatic neuroblastoma. This work has the potential for immediate clinical translation and could yield significant efficiencies for manufacturing of autologous engineered immune cell products.

### Results

HITI Outcompetes HDR for Knock-In into Human T Cells

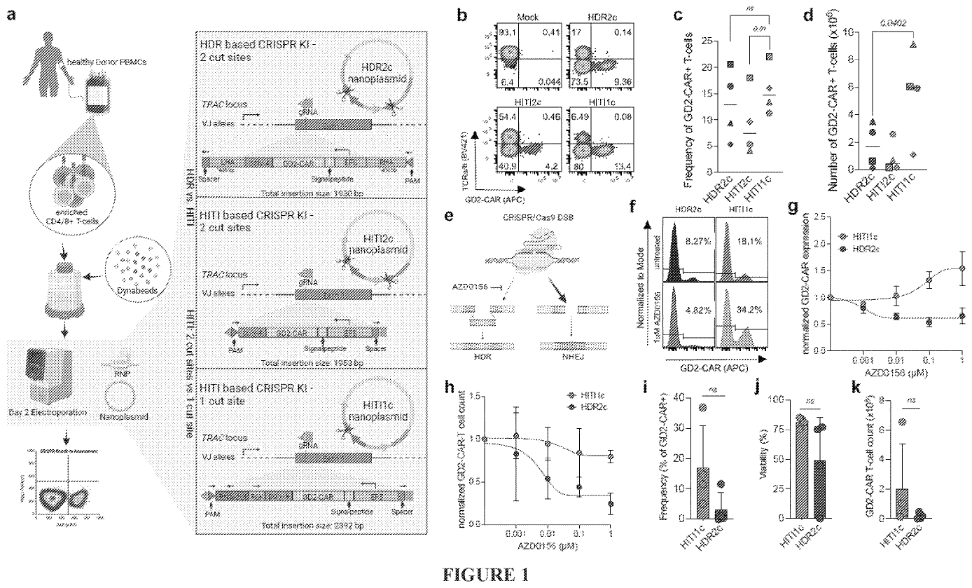

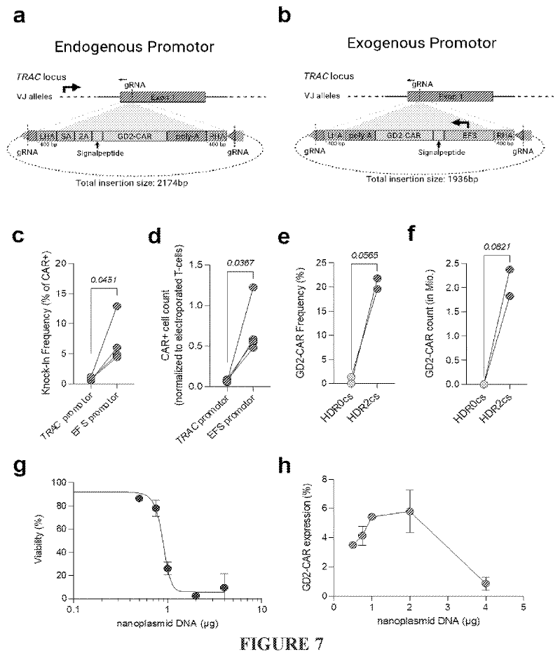

We compared insertion efficiency of HITI vs. HDR using plasmid DNA to deliver a GD2-CAR sequence into TRAC, which has been credentialled as a locus for targeted insertion (FIG. 1 a)^{15,18,20,31}. To maximize insertion efficiency and CAR expression in this system, we first optimized several elements. To determine whether the endogenous TRAC promoter is sufficient to drive GD2-CAR transgene expression, we used HDR mediated knock-in of a plasmid containing a splice acceptor followed by a 2A cleavage site and the desired GD2-CAR as previously described¹⁸ or a plasmid harboring an EF-1α short (EFS) promoter followed by the GD2-CAR. To prevent competition between the EFS and the endogenous TRAC promoter, the second construct was designed on the reverse strand (FIG. 7 a and b)¹⁸. Both flow cytometry and GD2-CAR T cell counts indicated enhanced expression using an exogenous promoter (FIG. 7 c and d). Next, we assessed the optimal number of cut sites for HDR and HITI constructs since both no (HDR0c) and two cut sites (HDR2c) have been proposed^{21,31}. Two cut sites were effective whereas HDR0c did not result in any detectable CAR insertion (FIG. 7 e and f). We also demonstrated that 0.75 μg plasmid DNA per 1×10⁶ electroporated T cells maximized knock-in efficiency without impairing T cell viability (FIG. 7 g and h), consistent with previous data using 0.5-1 μg linearized DNA per 1×10⁶ electroporated T cells¹⁵.

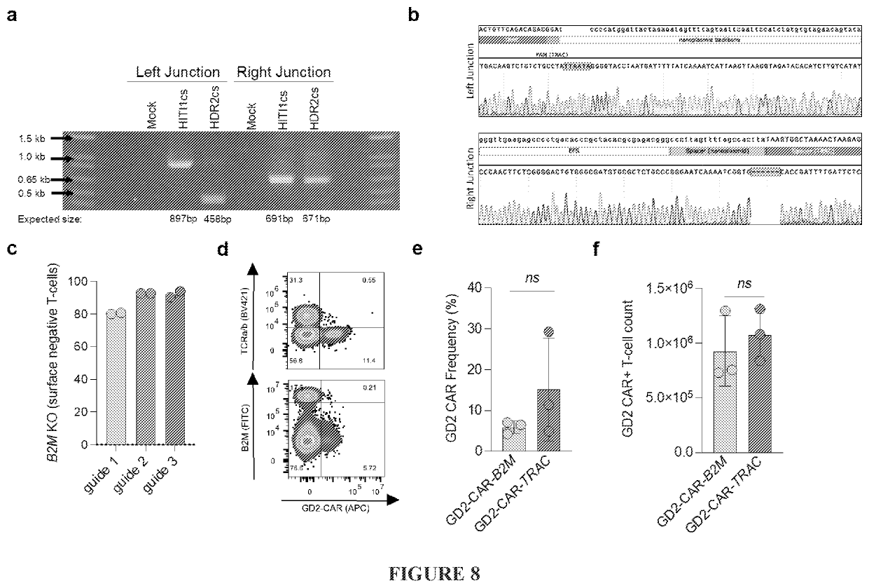

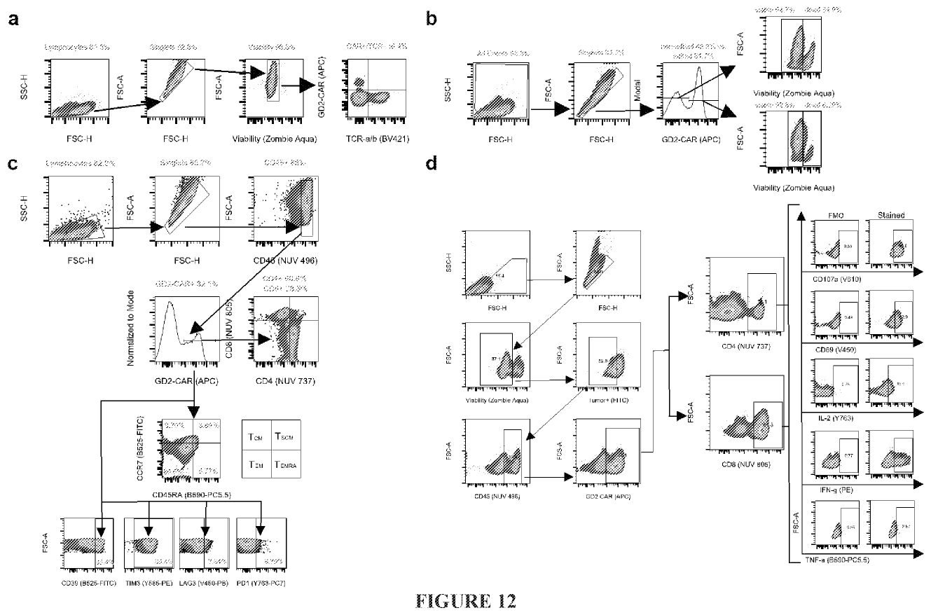

Using an optimized nanoplasmid DNA concentration, we compared HDR vs. HITI mediated knock-in. For HITI we used two iterations with either one (HITI1c) or two cut sites (HITI2c). Consistent with previous reports, one cut site resulted in higher knock-in frequencies (mean frequencies HITI1c=15.7% vs. HITI2c=9.3%, p=0.01) and yielded ~6-fold more GD2-CAR T cells^{23,26}. On average, we observed ~3-fold higher GD2-CAR T cell yields and comparable insertion rates when using HITI1c vs. HDR2c (FIG. 1 b-d, FIG. 12 a). We confirmed on target insertion of HDR2c and HITI1c constructs using IN&OUT PCR, which showed successful integration of the HITI1c plasmid construct with 6 bp Indels at both junctions (FIG. 8 a and b).

Next, we aimed to characterize HITI1c vs. HDR2c by modifying the experimental context. To promote HITI, we incubated T cells post electroporation with different concentrations of the ATM kinase inhibitor AZD0156, which has been described to inhibit HDR³². We observed decreased knock-in efficiency of HDR2c when AZD0156 was added, but higher insertion rates for HITI1c, validating the reliance on NHEJ in the HITI system. Nonetheless, we observed decreased GD2-CAR T cell yields in HITI1c samples after AZD0156 treatment compared to untreated controls (FIG. 1 f-h), and thus chose not include AZD0156 in the following experiments.

Given the cell cycle independent activity of the NHEJ repair pathway, we compared targeted genomic integration of the GD2-CAR using HITI1c and HDR2c in non-activated T cells and found higher insertion rates, viability and CAR+ T cell yield after knock-in with HITI1c (FIG. 1 i-k). To validate that HITI mediated knock-in is versatile, we explored knock-in of our GD2-CAR into B2M (FIG. 8 c-f). resulting in GD2-CAR T cell yields comparable to targeted insertion into TRAC.

### Optimization of CEMENT Using Clinical Grade Enrichment

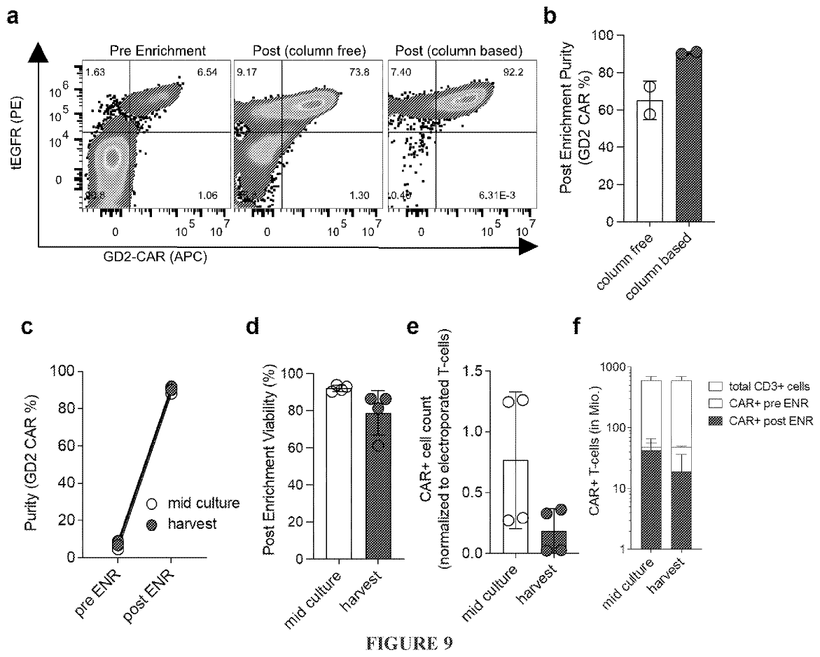

To enhance purity of HITI GD2 knock-in CAR-T cells, we optimized CEMENT by comparing three potentially clinically applicable enrichment systems, which incorporate human proteins to diminish the risk of immunogenicity and utilize GMP grade reagents or clinically approved drugs. We tested immunomagnetic enrichment targeting tEGFR and tNGFR as previously described^{9,33}, and drug based selection of cells expressing DHFR-FS, which confers resistance to the approved drug methotrexate (MTX)^{34,35}. To optimize conditions for surface marker based selection we compared column based versus column free magnetic selection and optimized the timing of surface marker based enrichment. We noted higher purities using a column based magnetic selection (FIG. 9 a and b) and higher viabilities and yields when enriching on day 9 of our culture (FIG. 9 c-f).

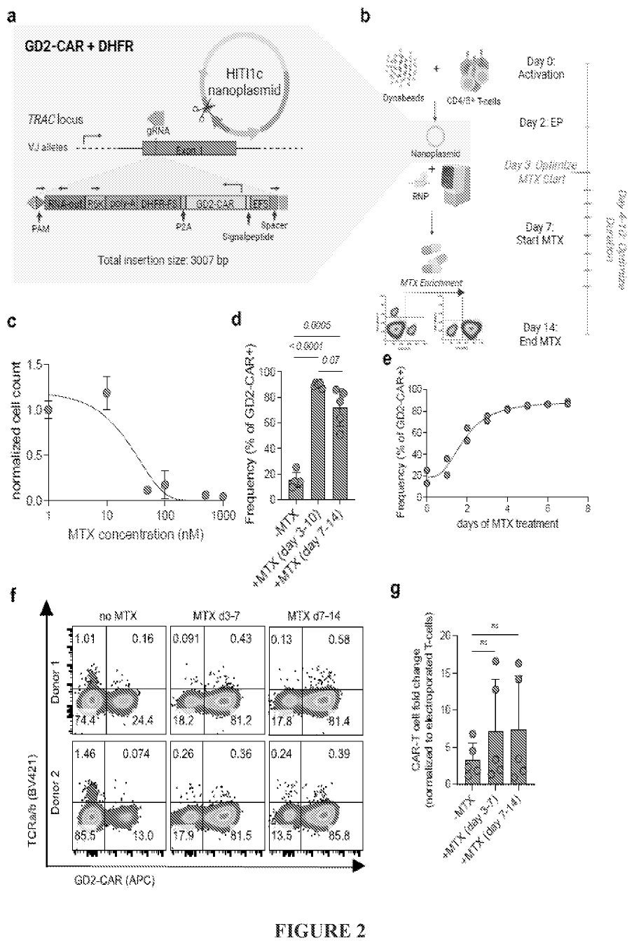

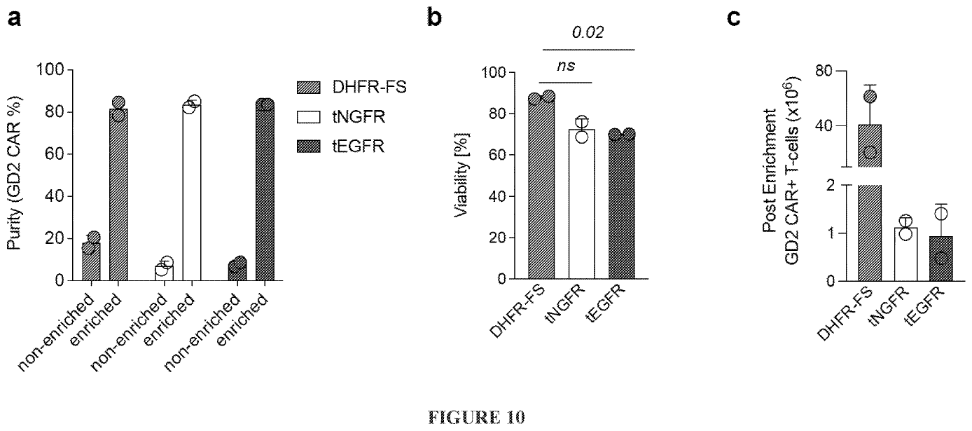

We next optimized timing and duration of selection using MTX (FIG. 2 a and b), first by exposing primary human T cells post activation to varying concentrations of MTX and confirming that MTX exerts cytostatic effects in dividing T cells at 50 nM (FIG. 2 c). We added MTX to the test condition on day 3 or day 7 as reported previously³⁴ (FIG. 2 b) and observed improved enrichment of GD2-CAR-T cells when MTX was added on day 3 (range 86.6-91.4%) instead of day 7 (range 50.8-85.7%) (FIG. 2 d). We next shortened the duration of MTX exposure, and observed increased GD2-CAR frequencies with treatment durations of up to 4 days, again resulting in ^{~}80% purity (range 76-81.5%). followed by a plateau for days 5-7 (FIG. 2 e). We next compared MTX based enrichment (optimized day 3-7 schedule) with surface marker based enrichment using tEGFR or tNGFR of GD2 knock-in CAR-T cells. The platforms were comparable in terms of purity and viability assessed 14 days after T cell activation. However, we observed ^{~}40-fold higher GD2-CAR-T cell recovery following DHFR-FS based enrichment (FIG. 10 a-c). Together, our optimized CEMENT procedure using MTX enrichment of GD2-CAR-DHFR-FS knock-in cells comprises a 14-day process to manufacture feeder-cell-free, non-viral CRISPR knock-in CAR-T cells, and shortened MTX exposure compared to previously published schedules for enrichment of viral transduced and megaTAL/AAV6 modified T-cells^{49, 51} (FIG. 2 f and g).

### HITI and CEMENT Enable Dose Relevant Manufacturing

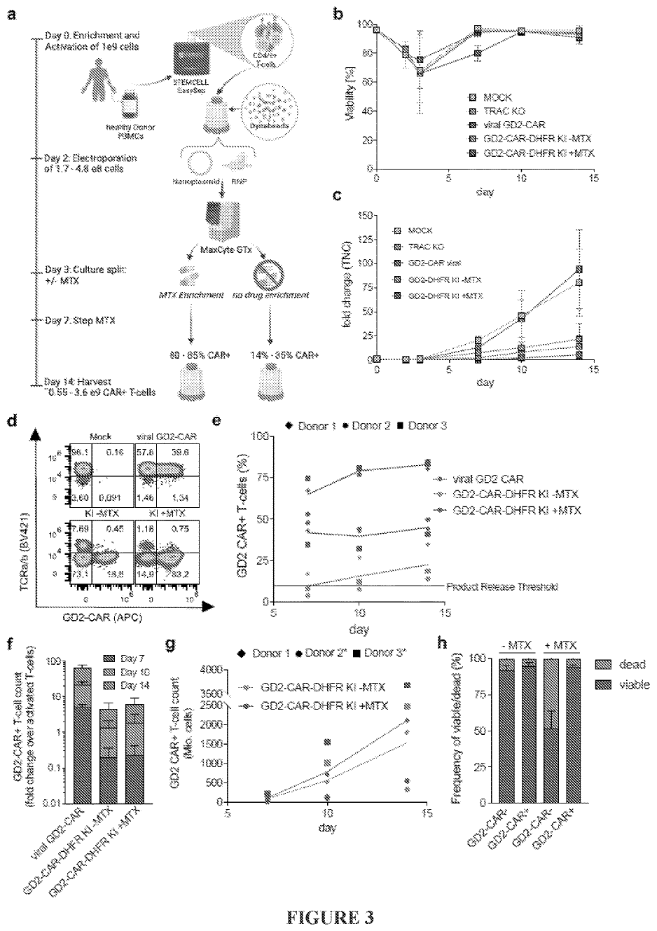

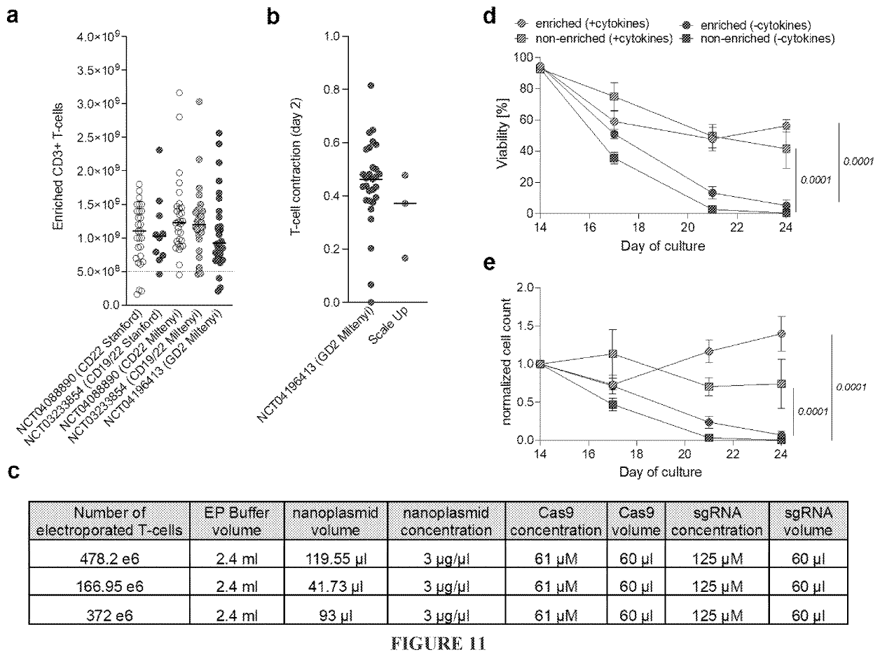

Given the high frequencies and yield achieved using HITI mediated CAR knock-in and DHFR-FS based enrichment at laboratory scale, we tested our novel manufacturing platform at clinical scale. Based on the numbers of enriched T cells obtained from processed leukapheresis from diseased patients treated on ongoing clinical trials at our institutions (NCT04196413, NCT04088890, NCT03233854)^{5,36,37} (FIG. 11a), we activated 1×10⁹ enriched T cells using CD3/CD28 Dynabeads and cultivated T cells in G-Rex. On day 2. activated T cells were electroporated using Maxcyte's GTx with the GMP compatible CL1.1 processing assembly to knock-in the GD2-CAR-DHFR-FS HITI1c construct into TRAC. T cell counts on day 2 prior to electroporation ranged from 1.7-4.8×10⁸ due to contraction and overlapped with historical day 2 cell counts obtained from patients treated on NCT04196413 which uses a similar activation process prior to retroviral vector based GD2-CAR delivery (FIGS. 11b and c)⁵. On day 3, HITI knock-in GD2-CAR-T cells were equally split and maintained in media containing 50 nM MTX or no drug. On day 7, media was replaced in the MTX enriched cultures and CAR-T cells were harvested on day 14 (FIG. 3 a). Samples obtained during manufacturing revealed that cell viabilities recovered to >90% by day 7 in all conditions apart from MTX enriched cultures, where cell viability reached >90% by day 10 (FIG. 3 b). Total fold change of T cells indicated expansion in edited cell samples (FIG. 3 c).

Flow cytometry of cells analyzed on days 7, 10 and 14 showed targeted insertion with HITI GD2-CAR-T cells and successful enrichment after MTX treatment (FIG. 3 d). On day 14, CAR frequencies ranged from 14-35% (non-enriched) and 80-85% (enriched) respectively (FIG. 3 e) with 3-4 fold expansion of HITI GD2-CAR-T cells compared to the number of initially activated T cells, on average resulting in a total of 2.1×10⁹ (range: 5.5×10⁸-3.6×10⁹) GD2-CAR-T cells in the enriched cultures (FIG. 3 f and g). These results demonstrate successful scale up of HITI mediated targeted transgene insertion and CEMENT. Last, we explored the effects of MTX on viability of edited vs. non-edited T cells using a reversed gating strategy (FIG. 12 b) and observed that only non-edited cells were compromised in their viability at the end of MTX treatment (day 7) (FIG. 3 h), demonstrating that DHFR-FS successfully confers resistance against MTX. In addition, an extended culture using media either supplemented with or without cytokines (IL-7+IL-15) did not show any cytokine independent outgrowth of DHFR-FS expressing cells independent of MTX exposure (FIG. 11 d and e).

### Functional Comparison of Knock-In and Transduced CAR-T Cells

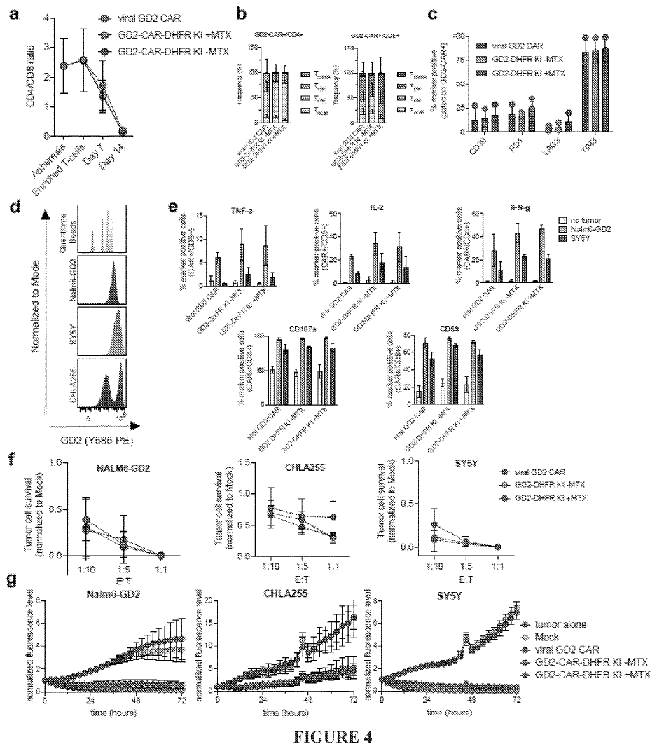

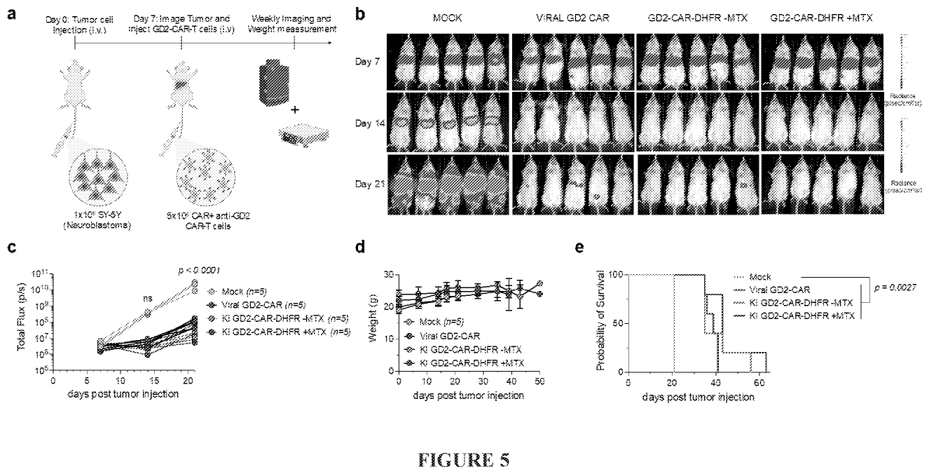

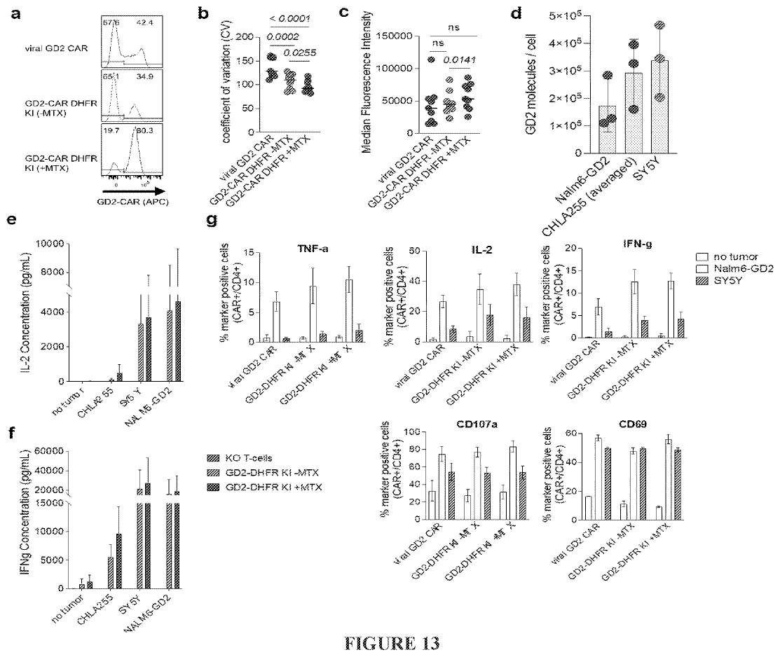

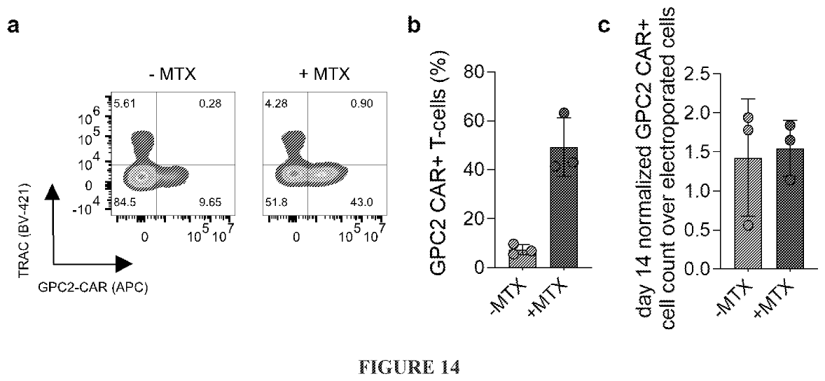

To understand how knock-in compares to viral transduced GD2-CAR-T cells, we analyzed CD4/8 ratio, memory phenotype and exhaustion profile and observed no significant differences (FIG. 4 a-c, FIG. 12 c). We observed a clear bimodal distribution of CAR expression for the knock-in CAR-T cells with a slightly higher MFI and a significantly lower coefficient of variation compared to virally transduced GD2-CAR-T cells (FIG. 13 a-c). To compare functionality of GD2-CAR-T cells generated by viral transduction vs HITI/CEMENT, we performed co-culture experiments using GD2+ tumor cell lines (FIG. 4 d, FIG. 13 d), and observed similar activation marker expression, cytokine production and tumor cell killing at low E:T ratio (1:10) by viral and knock-in GD2-CAR-T cells (FIG. 4 e-g, FIG. 13 e-g). We next tested performance of HITI/CEMENT CAR-T cells in an in vivo model of metastatic neuroblastoma. We injected 1×10⁶ SY5Y tumor cells via tail vein injection and confirmed tumor lesions in liver and bone marrow on day 7. Hereafter, 5×10⁶ CAR+ T cells were injected via tail vein injection. Tumor burden was tracked by weekly live imaging and weight measurements to assess toxicity (FIG. 5 a). While Mock T cell treated tumors did not show any disease control, all treatment arms showed comparable and significantly delayed tumor growth without evidence of toxicity (FIG. 5 b-e). Furthermore, we extended our work to generate Glypican-2 (GPC2) knock-in CAR-T cells using HITI/CEMENT resulting in knock-in efficiencies ranging from 5.5%-9.7% for non-enriched and from 41.4%-63.2% for enriched GPC2 knock-in CAR-T cells as determined on day 10 (FIG. 14 a and b). GPC2 knock-in CAR-T cell counts showed a mean 1.54-fold increase over electroporated enriched T-cells by day 14 (FIG. 14 c).

### Off-Target Genomic Toxicity Assessment in Knock-In CAR-T Cells

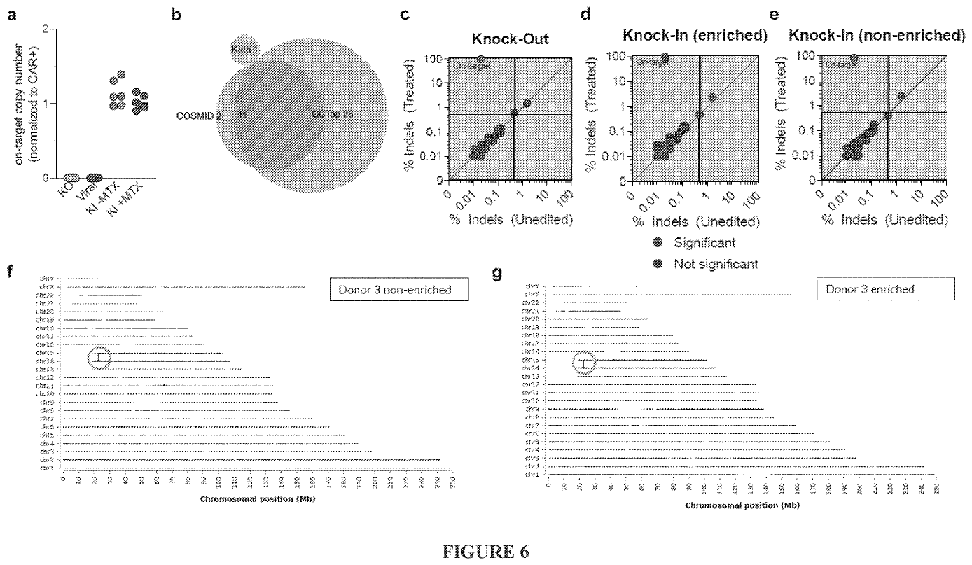

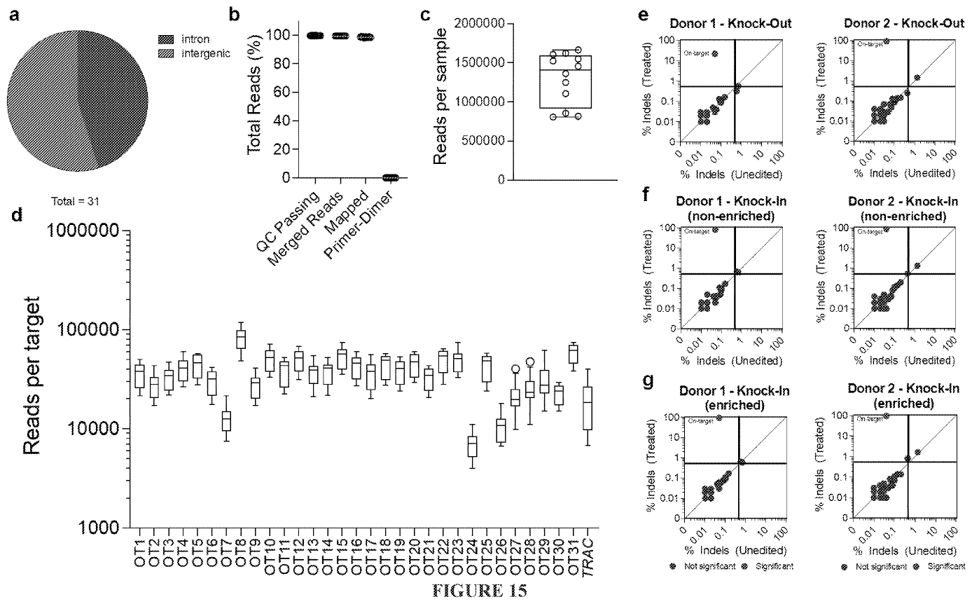

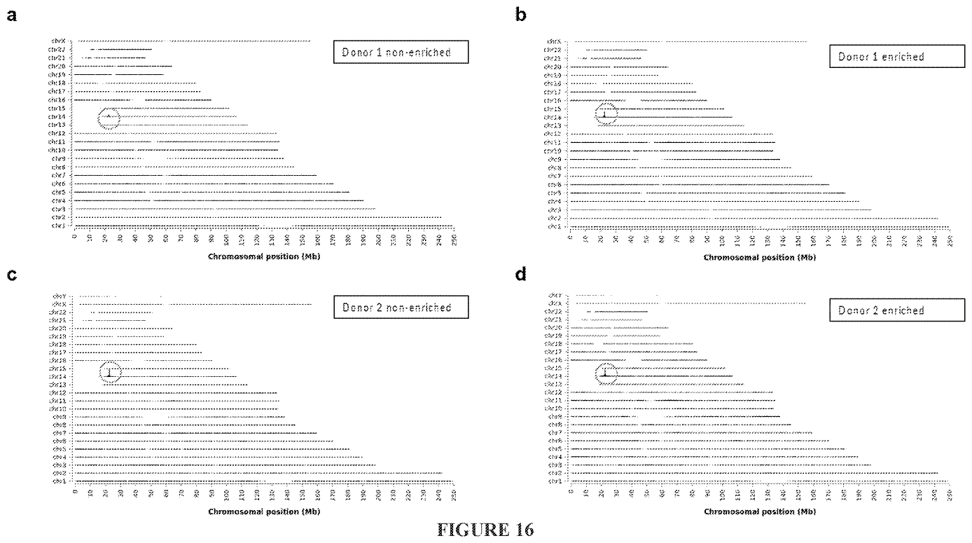

We assessed copy number levels of GD2-CAR utilizing ddPCR, which showed an average copy number for GD2 knock-in CAR-T cells of 1.08 (FIG. 6 a). To identify off-target cut sites introduced by the CRISPR/Cas9 RNP, we utilized a gRNA previously optimized for off-target cutting as assessed via GUIDE-Seq¹⁵ and we also predicted additional off-target cut sites by applying the in silico prediction tools COSMID and CCTop^{38,39} (FIG. 6 b). All predicted cut sites are located outside exonic sequences (FIG. 15 a) and were quantified using rhAmpSeq³⁰. We conducted off-target evaluation in Mock, Knock-out, Knock-In-MTX and Knock-In+MTX samples of all three donors studied during our large-scale experiment. Quality control confirmed high quality sequencing data with a median coverage of 36,090× and 99.1% of sequenced sample sites exceeding 5,000× coverage (FIG. 15 b-d). To classify off-target editing binarily, we applied a thresholded Fishers Exact test on paired treatment/control data with a limit of detection of 0.5% indels, as previously described¹⁵ and compared edited samples against Mock control conditions for each respective donors. We identified significant and biologically relevant editing at the on-target site, i.e. TRAC, but no off-target editing was detected, indicating that neither CRISPR knock-in via HITI nor the MTX based enrichment procedure increased the risk of off-target editing (FIG. 6 c-e, FIG. 15 e-g). To confirm that our approach results in site directed knock-in, we performed targeted locus amplification (TLA) as described previously³³ using non-enriched and enriched samples from all three large scale runs. This unbiased genome-wide insertion site analysis has been widely accepted in the field^{20, 52} and confirmed that our HITI/CEMENT approach inserts without off-target integration (FIG. 6 f and g, FIG. 16).

### DISCUSSION

This work provides a new approach for clinical scale manufacturing to deliver CAR knock-in to human T cells using a fully non-viral CRISPR/Cas9 based platform. Here, we show for the first time that plasmid DNA mediated HITI is feasible for targeted transgene insertion into primary human T cells and demonstrate that HITI combined with CEMENT results in clinically relevant CAR-T cell yields, providing an efficient and genotoxicity-free clinical scale manufacturing process (FIG. 1 d, FIG. 8 b, FIGS. 3 and 6). The process utilizes reagents available in sufficient quantities at reasonable cost to support both early proof-of-concept trials and potentially commercial manufacturing of CAR-T cell products. Our approach utilized plasmid DNA as a delivery platform, which based upon our experience with research grade templates provided from vendors, is available at least 4-times quicker than dsDNA and is ^{~}20-fold cheaper. Furthermore, a recent report indicates plasmid DNA to be more efficient in transgene delivery than dsDNA³¹. Plasmid DNA can be manufactured in batches beyond 1 g, raising the prospect that one batch of plasmid could provide sufficient template DNA to manufacture CRISPR knock-in CAR-T cells for a few thousand patients⁴⁰. A clinical trial (NCT03970382) published by Foy et al. indicated the feasibility of utilizing nanoplasmid DNA as donor template within a clinical manufacturing context. The authors used nanoplasmid DNA to manufacture non-viral CRISPR knock-in neoantigen-specific TCR (neoTCR) T-cell products via HDR, which after optimizing their manufacturing process and incorporating a pre-commercial electroporation device, resulted in acceptable knock-in frequencies and yields of their CD8+ neoTCR T-cells⁵².

Here, we used a HDR construct with two internal RNP cut sites as has been proposed recently^{21,31}. During our studies, two other groups however published data, successfully utilizing nanoplasmid DNA based HDR templates without internal RNP cut sites^{47,52}. When comparing optimized HITI vs. HDR in our system utilizing internal template cut sites and an external promoter for comparable transgene expression between HITI and HDR templates, we observed that insertion efficiencies were more consistent with HITI1c and independent of the cell cycle state based upon similar efficiencies with activated and non-activated T cells (FIG. 1 b, c, i). Further, yield of GD2-CAR-T cells was higher when using HITI1c versus HDR2c (FIG. 1 d and k). The basis for this finding remains unclear, but could be explained by the mechanism of transgene insertion. While for HITI the plasmid DNA provided during electroporation is incorporated directly into the genome, for HDR the plasmid DNA serves as a template for the endogenous HDR machinery and leaving remaining post-insertion exogenous HDR plasmid DNA that could exert toxic effects on successfully edited T cells and thereby reducing CAR-T cell yield (FIG. 8 a and b). An alternative approach to improve CAR-T cell yields has recently been proposed²³ and relies on the combination of tCTS⁵³ and ssDNA donor templates, which are known to be less toxic than dsDNA templates²⁰. By adding tCTS to the ends of ssDNA templates, RNP will bind to the ssDNA and via the nuclear localization sequence of Cas9 promote shutting of the donor DNA into the nucleus helping to improve the historically low insertion efficiencies of ssDNA. However, tCTS have not been successful when integrated into donor DNA templates of other groups^{15,47}, and we anticipate that access to large yields of ssDNA will be limited.

Our data also demonstrates that delivery of DHFR-FS protein provides a powerful tool for MTX based enrichment of edited T cells, generating GD2 CAR-T cell products with purities of at least 75% and confirms that post editing DHFR-FS expression renders primary human T cells resistant to nanomolar doses of MTX as has been shown with megaTAL/AAV6 edited human CD4+ T-cells enriched for cells successfully edited at the CCR5 locus (FIG. 3 h)⁵¹. While our scale up work used healthy donors for manufacturing, the contraction seen on day 2 across all three donors used in our study aligned well with changes in cell numbers observed using viral-mediated CAR-T cell manufacturing on day 2 within NCT04196413⁵ (FIG. 12 b). Of interest, one of the donors used in our study showed a massive contraction resulting in only 17% of activated T cells remaining prior to electroporation (FIG. 12 c). Despite this, the HITI/CEMENT approach yielded a clinically relevant number of CAR-T cells for this donor (FIG. 3 g). Together, the data demonstrate that HITI CAR-T cells are comparable in function to viral GD2-CAR-T cells (FIGS. 4 and 5), do not show significant evidence for genotoxicity and are feasible to support clinical-scale manufacturing (FIG. 6 and FIGS. 15 and 16).

In summary, this data demonstrates that HITI expands the toolbox for precise genome engineering of human T cells. HITI delivered higher yields of anti-GD2-CAR-T cells sufficient for clinical application. We chose nanoplasmid DNA as our delivery platform due to its lower cost per product compared to linearized DNA templates. Our approach provides new avenues to explore HITI within the context of activator-free ex vivo manufacturing of CAR-T cells or in situ generation of CAR-T cells^{54,55}.

### Methods

### Isolation and Culture of Primary Human T Cells

Fresh Leukopaks were obtained from STEMCELL Technologies and processed for negative selection using the EasySep Human T Cell Isolation Kit. Cells were activated using Dynabeads Human T-Activator CD3/CD28 (Thermo Fisher) at a 1:1 ratio and cultivated in TexMACS media supplemented with human IL-7 at 12.5 ng/ml and IL-15 at 12.5 ng/ml (all Miltenyi Biotec) as well as 3% human male AB Serum (Access Cell Culture). The culture volume was expanded over time to maintain cells at a concentration of ^{~}1.5×10⁶/ml using G-Rex 24-well and 6-well plates or G-Rex 100M according to manufacturer's instructions (Wilson-Wolf). Small molecule inhibitors AZD0156 (Selleck Chemicals) and Methotrexate (Sigma Aldrich) were resuspended in DMSO and added to the cell culture media as indicated.

### Plasmid Design and Production

Plasmid DNA was optimized for gene therapeutic application and consisted of two components: R6K origin of replication and an anti-Levansucrase antisense RNA to allow for an antibiotic-free selection. This ^{~}430 bp backbone prevents transgene silencing after genomic insertion^{40,43,44}. Knock-In templates were synthesized at Genscript and flanking cut sites for NheI and KpnI were used to clone synthetic genes into plasmid backbones. To introduce RNP cut sites within the plasmid DNA, the genomic target of the respective RNP was included into the above mentioned synthetic gene.

sgRNA Design

All gRNA sequences used in this study have been previously published^{15,16,45,46} and target: TRAC 5′-GGGAATCAAAATCGGTGAAT-3′, instead of 5-GAGAATCAAAATCGGTGAAT-3′, 5′-TCAGGGTICTGGATATCTGT-3′ and B2M 5′-CGCGAGCACAGCTAAGGCCA-3′, 5′-GAGTAGCGCGAGCACAGCTA-3′, 5′-GGCCGAGATGTCTCGCTCCG-3′

### Electroporation

On day 2, unless otherwise indicated, Dynabeads were magnetically removed and cells were counted prior to electroporation on the Maxcyte GTx. For electroporation cells were washed once in Electroporation Buffer (Maxcyte) and then resuspended at 2×10⁸/ml for small scale experiments or in 2.4 ml for large-scale electroporation. Wildtype Cas9 (61 μM, IDT) and sgRNA (125 μM, IDT) were mixed vol 1:1 resulting in a molar ratio of 2:1 and incubated for 10 min. at room temperature. Hereafter indicated amounts of plasmid DNA (3 mg/ml) were added to the RNP and incubated for at least 10 min. to allow the RNP to cut the plasmid DNA. For small-scale experiments 5×10⁶ T cells were resuspended in 25 μl and 1.25 μl of RNP and respective amounts of plasmid DNA were added. Cells were then transferred into OC-25×3 processing assemblies and electroporated using the Expanded T cell 4 protocol for activated T cells or the Resting T cell 14-3 protocol for electroporation of non-activated T cells, which were stimulated after electroporation with Dynabeads at a 1:1 ratio. Small scale experiments for GPC2 CAR knock-in were conducted using the OC100×2 processing assembly with a final reaction volume of 100 μl. For large scale experiments the GMP compatible CL1.1 assembly was used. Post electroporation cells were rested in electroporation buffer either in the processing assembly (OC-25×3, OC-100×2) or in G-Rex 6-well plates (CL1.1) for 30 min. before being transferred into final G-Rex vessels.

### Viral Transduction

Our clinical grade GD2-CAR retroviral vector⁵ was spinoculated on Retronectin (Takara) coated plates for 2 hours at 3200 rpm on day 2. Hereafter, Dynabeads were removed from activated T cells and T cells were transduced at a MOI of 10 for 24 hrs.

### Post Editing CEMENT

To enable enrichment of the desired CAR+ knock-in population we optimized CEMENT based selection by comparing three different clinically relevant enrichment markers: Dihydrofolate Reductase^{L22F/F31S} (DHFR-FS), truncated Epidermal Growth Factor Receptor (tEGFR), and truncated Nerve Growth Factor Receptor (tNGFR)^{9,17,33}. All three enrichment markers were successfully co-inserted along with the GD2-CAR resulting in total transgene sizes of ^{~}2.5-3 kb. CRISPR knock-in CAR-T cells were either enriched using the FDA approved drug MTX which was diluted from a 10 mM stock in media to result in a 50 nM final concentration. Successfully edited cells expressing the DHFR-FS protein are resistant to MTX while cells that did not harbor the CAR transgene stop proliferating. For comparison of column-based versus column-free surface marker selection GD2-CAR-tEGFR+ cells were incubated with a primary antibody targeting tEGFR (Biolegend, clone: AY13) conjugated to Biotin. Hereafter successfully labeled T cells were either enriched using LS-columns and the QuadroMACS magnetic column separation system along with Streptavidin Microbeads (all Miltenyi Biotec) or using the EasySep Biotin Positive Selection kit for column-free separation (STEMCELL Technologies).

### Cell Counts and Viability

Cell counts and Viability were obtained using the Nexcelom Cellometer Auto 2000. Samples were mixed with AO/PI dye at a 1:1 volume ratio and then analyzed using the setting Immune Cells—Low RBC.

### Flow Cytometry and Intracellular Staining

For flow cytometry 3-5×10⁵ cells were stained for 30 min. at 4 C using commercially available antibodies as listed in Table 1. For GD2-CAR and GPC2-CAR detection, an anti-14g2a idiotype or recombinant human Glypican-2 protein (R&D systems) were fluorescently labeled with the DyLight 650 Microscale Antibody Labeling kit (Thermo Fisher). For intracellular staining CAR+ T cells were co-cultured with respective tumor cell lines at a 1:4 ratio. Prior to co-cultivation the protein transport inhibitor Monensin (BD) and the anti-CD107a antibody (Biolegend) were added to T cell samples. 6 hours after co-culture a surface staining was performed (30 min. at 4 C), hereafter co-cultured cells were fixed for 50 min. at 4 C and permeabilized to allow for intracellular staining over night at 4 C using the Fixation/Permeabilization Solution Kit (BD Biosciences) as described previously³⁷. Flow cytometry was performed on the CytoFLEX LX (Beckman Coulter).

TABLE 1
Antibodies used in the disclosure:

| Antibodies | Clone | Fluorophore | Source | Identifier |
|---|---|---|---|---|
| Viability dye | NA | Zombie Aqua | Biolegend | cat. no. #423102 |
| TCR-a/b | IP26 | BV421 | Biolegend | cat. no. #306722 |
| Anti-14G2A | 1A7 | APC | NIH | Sen et al., 1998 |
| idiotype antibody | | | | |
| EGFR | AY13 | PE/Biotin | Biolegend | cat. no. |
| | | | | #352904/3352934 |
| NGFR | ME20.4 | PE/Biotin | Biolegend | cat. no. |
| | | | | #345106/#345122 |
| CD4 | OKT4 | BU737 | BD | cat. no. #750977 |
| | | | Biosciences | |
| CD8 | SK1 | BUV805 | BD | cat. no. |
| | | | Biosciences | #564912/612889 |
| CD45 | HI30 | BUV496 | BD | cat. no. #750179 |
| | | | Biosciences | |
| CD45RA | HI100 | PerCP | Biolegend | cat. no. #304156 |
| CCR7 | G043H7 | FITC | Biolegend | cat. no. #353216 |
| CD39 | A1 | FITC | Biolegend | cat. no. #328206 |
| PD1 | EH12.1 | PE-Cy7 | BD | cat. no. 561272 |
| | | | Biosciences | |
| LAG3 | 11C3C65 | BV421 | Biolegend | cat. no. #369314 |
| TIM3 | F38-2E2 | PE | Biolegend | cat. no. #345006 |
| CD107a | H4A3 | BV605 | Biolegend | cat. no. #328634 |
| CD69 | FN50 | BV421 | Biolegend | cat. no. #310930 |
| IFN-g | 4S.B3 | PE | Biolegend | cat. no. #502509 |
| IL-2 | MQ-17H12 | PE-Cy7 | Biolegend | cat. no. #500326 |
| TNF-a | MAb11 | PerCP | Biolegend | cat. no. #502924 |

### In Vitro Killing and ELISA Assays

GD2-CAR-T cells from scale up experiments were harvested and co-cultured in 96-well plates at a 1:1, 1:5 and 1:10 ratio (normalized for CAR+ T cells) with tumor cell lines Nalm6-GD2, CHLA255, SY5Y, or SMS-SAN expressing GFP using 50,000 cells. Co-cultures were monitored for fluorescence signal using the Incucyte (Sartorius). For ELISA assays co-cultures were conducted at a 1:1 ratio and supernatant was collected 24 hours later after centrifugation at 300 g for 10 minutes. ELISA MAX human IL-2 and IFN-g kits were purchased from Biolegend and co-culture supernatants were processed according to manufacturer's instructions. Hereafter, plates were analyzed using the Thermo Scientific Varioskan Lux.

### Tumor Cell Line Culture and In Vivo Studies

Nalm6-GD2, CHLA255, SY5Y, and SMS-SAN tumor cell lines were cultivated in RPMI (Gibco) supplemented with 10% FBS (Sigma Aldrich) and 1% Penicillin/Streptomycin (Gibco). For in vivo studies SY5Y tumor cells were resuspended at 5×10⁶/ml in PBS and 200 μl (=1×10⁶ tumor cells) were injected via tail vein injection into six- to ten-week-old female NOD-SCID γ^{c-/-} (NSG) mice. Mice were bred in house under Stanford University APLAC-approved protocols as described previously⁴⁷. 7 days later 5×10⁶ GD2-CAR+ T cells were injected via tail vein injection. Mice were euthanized when they manifested hunched posture, persistent scruffy co unless at, paralysis, impaired mobility, greater than 20% weight loss, if tumors interfered with normal bodily functions or if they exceeded limits designated in APLAC-approved protocols. Firefly luciferase expression was used to detect tumor activity over time. Bioluminescence images were taken after administration of 3 μg of D-Luciferin (15 μg/ml) by intraperitoneal injection. Images were either acquired on an IVIS imaging system 5 min. after injection using 30 sec exposure times and medium binning or applying the auto-exposure setting. For data analysis all images were collected in a single file and analyzed on the Living Image software (Perkin Elmer).

### Genomic DNA Extraction, IN&OUT PCR and Sanger Sequencing

Genomic DNA was extracted using the PureLink Genomic DNA Mini Kit (Invitrogen). Primers flanking the insertion sites of HITI1c and binding outside the respective homology arm sequence of HDR2c as well as primers binding within the inserted sequences were designed using Primer3Plus (www.primer3plus.com). Phusion Hot Start Flex 2× Mastermix (NEB) was used to amplify genomic regions of interest and DNA gel electrophoresis was conducted using the E-Gel Power Snap System (Thermo Scientific). Samples were shipped to ELIM BIOPHARM for Sanger sequencing. Sequences were aligned for analysis using snapgene.

Post Enrichment and in Process Cell Counts from Clinical Products

In process cell counts were derived from post enrichment or day 2 samples of patients treated within NCT04196413, NCT04088890 and NCT03233854^{5,36,37}. Manufacturing was either conducted in-house at the Stanford Laboratory for Cell and Gene Medicine (LCGM) or at Miltenyi Biotec. All clinical studies and their amendments were approved by the Stanford University Institutional Review Board.

ddPCR and Copy Number Assessment

Genomic DNA was extracted using the PureLink Genomic DNA Mini Kit (Invitrogen). Primers flanking the insertion sites of HITI1c and binding outside the respective homology arm sequence of HDR2c as well as primers binding within the inserted sequences were designed using Primer3Plus (www.primer3plus.com). Phusion Hot Start Flex 2× Mastermix (NEB) was used to amplify genomic regions of interest and DNA gel electrophoresis was conducted using the E-Gel Power Snap System (Thermo Scientific). Samples were shipped to ELIM BIOPHARM for Sanger sequencing. Sequences were aligned for analysis using snapgene.

In process cell counts were derived from post enrichment or day 2 samples of patients treated within NCT04196413. NCT04088890 and NCT03233854^{5,36,37}. Manufacturing was either conducted in-house at the Stanford Laboratory for Cell and Gene Medicine (LCGM) or at Miltenyi Biotec. All clinical studies and their amendments were approved by the Stanford University Institutional Review Board.

Extracted genomic DNA was digested using HindIII (NEB) and 10-66 ng of digested DNA were analyzed per sample. Samples were prepared using the Bio-Rad ddPCR Supermix for probes (no dUTP). Primer/Probe assays for Albumin (reference) and for detection of the TRAC-GD2-CAR insertion site (target) were designed using Primer3Plus (www.primer3plus.com) and purchased from IDT. For thermal cycling conditions we followed vendor recommendations (Bio-Rad, #1863024) and used an optimized annealing temperature at 58° C. Droplets were generated using the QX200 manual droplet generator (Bio-Rad) and analyzed post PCR in the QX200 Droplet Reader (Bio-Rad). Samples were analyzed with QX Manager software using the automated threshold for digital cut-off. Copy numbers were normalized to the Albumin reference as well as GD2-CAR % and calculated as follows:

$$normalized CN = \frac{target copies \times 2}{reference copies \times fraction of CAR expressing cells}$$

### Off-Target Site Prediction and Quantification

Genomic DNA was extracted using the PureLink Genomic DNA Mini Kit (Invitrogen). Primers flanking the insertion sites of HITI1c and binding outside the respective homology arm sequence of HDR2c as well as primers binding within the inserted sequences were designed using Primer3Plus (www.primer3plus.com). Phusion Hot Start Flex 2× Mastermix (NEB) was used to amplify genomic regions of interest and DNA gel electrophoresis was conducted using the E-Gel Power Snap System (Thermo Scientific). Samples were shipped to ELIM BIOPHARM for Sanger sequencing. Sequences were aligned for analysis using snapgene.

In process cell counts were derived from post enrichment or day 2 samples of patients treated within NCT04196413. NCT04088890 and NCT03233854^{5,36,37}. Manufacturing was either conducted in-house at the Stanford Laboratory for Cell and Gene Medicine (LCGM) or at Miltenyi Biotec. All clinical studies and their amendments were approved by the Stanford University Institutional Review Board.

Extracted genomic DNA was digested using HindIII (NEB) and 10-66 ng of digested DNA were analyzed per sample. Samples were prepared using the Bio-Rad ddPCR Supermix for probes (no dUTP). Primer/Probe assays for Albumin (reference) and for detection of the TRAC-GD2-CAR insertion site (target) were designed using Primer3Plus (www.primer3plus.com) and purchased from IDT. For thermal cycling conditions we followed vendor recommendations (Bio-Rad, #1863024) and used an optimized annealing temperature at 58° C. Droplets were generated using the QX200 manual droplet generator (Bio-Rad) and analyzed post PCR in the QX200 Droplet Reader (Bio-Rad). Samples were analyzed with QX Manager software using the automated threshold for digital cut-off. Copy numbers were normalized to the Albumin reference as well as GD2-CAR % and calculated as follows:

Off-target sites were either described previously¹⁵, or predicted using COSMID³⁸ and CCTop³⁹. For COSMID the algorithm was set to allow for 3 mismatches and 1 Indel as described by Wiebking et al.⁴⁸ Additional off-target sites were predicted using CCTop which was adjusted to allow for a total of 4 mismatches, with 2 mismatches being concatenated. Primer pools were designed for multiplex PCR of off-target sites using the rhAmpSeq Design Tool (IDT) (https://www.idtdna.com/rhampseqdesigntool) and respective sites were amplified using the rhAmpSeq library kit (IDT) according to manufacturer's instructions. Samples were pooled from all three independent donors to generate an equimolar ratio. The final sample was then sequenced at Novogene using MiSeq v2 chemistry (Illumina). Editing events were quantified using CRISPAltRations utilizing default Cas9 parameterization, as described previously^{15,49}. Binary classification of off-target editing was performed using a thresholded Fishers Exact Test (p<0.05) with limitations for site classification (% indels in treatment>0.5%; % indels in control<0.4%; >5,000 reads per site)¹⁵. Products and tools supplied by IDT are for research use only and not intended for diagnostic or therapeutic purposes. Purchaser and/or user are solely responsible for all decisions regarding the use of these products and any associated regulatory or legal obligations.

### Insertion Site Analysis

For whole genome mapping of anti-GD2 CAR integration, edited cells were crosslinked and shipped to Cergentis B.V. for digestion, reverse crosslinking with ligation, PCR, subsequent sequencing, and data analysis. NGS reads were aligned to the CAR sequence and the human genome (hg19 sequence) as described previously³³.

### Data Analysis and Software

Data analysis was performed using Microsoft Excel and GraphPad Prism. Statistical tests were conducted in GraphPad Prism and are indicated in the respective figure legend. Plasmid and gRNA sequences were designed and confirmed in SnapGene (Dotmatics). We used FlowJo (FlowJo LLC) to analyze .fcs files derived from flow cytometry experiments. Living Image (PerkinElmer) was used for analysis of images derived from in vivo treatment of mice. Schematic illustrations were created in BioRender.

### REFERENCES

1. Maude S L, Laetsch T W, Buechner J, Rives S, Boyer M, Bittencourt H, et al. Tisagenlecleucel in Children and Young Adults with B-Cell Lymphoblastic Leukemia. N Engl J Med. 2018:378(5):439-48.
2. Wang M, Munoz J. Goy A, Locke F L, Jacobson C A, Hill B T, et al. KTE-X19 CAR T-Cell Therapy in Relapsed or Refractory Mantle-Cell Lymphoma. N Engl J Med. 2020; 382(14):1331-42.
3. Munshi N C, Anderson L D, Shah N, Madduri D, Berdeja J, Lonial S. et al. Idecabtagene Vicleucel in Relapsed and Refractory Multiple Myeloma. N Engl J Med. 2021; 384(8):705-16.
4. Locke F L, Miklos D B, Jacobson C A, Perales M-A, Kersten M-J, Oluwole 00, et al. Axicabtagene Ciloleucel as Second-Line Therapy for Large B-Cell Lymphoma. N Engl J Med. 2021 Dec. 11; 386(7):640-54.
5. Majzner R G, Ramakrishna S, Yeom K W, Patel S, Chinnasamy H, Schultz L M, et al. GD2-CAR T cell therapy for H3K27M-mutated diffuse midline gliomas. Nature. 2022; 603(7903):934-41.
6. Theruvath J, Sotillo E, Mount C W, Graef C M, Delaidelli A. Heitzeneder S, et al. Locoregionally administered B7-H3-targeted CAR T cells for treatment of atypical teratoid/rhabdoid tumors. Nat Med. 2020; 26(5):712-9.
7. Srivastava S, Salter A I, Liggitt D, Yechan-Gunja S, Sarvothama M, Cooper K, et al. Logic-Gated ROR1 Chimeric Antigen Receptor Expression Rescues T Cell-Mediated Toxicity to Normal Tissues and Enables Selective Tumor Targeting. Cancer Cell. 2019 Mar. 18; 35(3):489-503.e8.
8. Larson R C, Kann M C, Bailey S R, Haradhvala N J, Llopis P M, Bouffard A A, et al. CAR T cell killing requires the IFNγR pathway in solid but not liquid tumours. Nature. 2022:604(7906):563-70.
9. Vitanza N A, Johnson A J, Wilson A L, Brown C, Yokoyama J K, Kiinkele A, et al. Locoregional infusion of HER2-specific CAR T cells in children and young adults with recurrent or refractory CNS tumors: an interim analysis. Nat Med. 2021; 27(9):1544-52.
10. Lyu L, Feng Y, Chen X, Hu Y. The global chimeric antigen receptor T (CAR-T) cell therapy patent landscape. Nat Biotechnol. 2020; 38(12):1387-94.
11. Ran T, Eichmiller S B, Schmidt P, Schlander M. Cost of decentralized CAR T-cell production in an academic nonprofit setting. Int J Cancer. 2020; 147(12):3438-45.
12. Kebriaci P, Singh H, Huls M H, Figliola M J, Bassett R, Olivares S, et al. Phase I trials using Sleeping Beauty to generate CD19-specific CAR T cells. J Clin Invest. 2016:1-14.
13. Magnani C F, Gaipa G, Lussana F, Belotti D, Gritti G, Napolitano S, et al. Sleeping Beauty-engineered CAR T cells achieve antileukemic activity without severe toxicities. J Clin Invest. 2020:130(11):6021-33.
14. Bishop D C, Clancy L E, Simms R, Burgess J, Mathew G, Moezzi L, et al. Development of CAR T-cell lymphoma in 2 of 10 patients effectively treated with piggyBac-modified CD19 CAR T cells. Blood. 2021 Oct. 21; 138(16):1504-9.
15. Kath J, Du W, Pruene A, Braun T, Thommandru B. Turk R, et al. Pharmacological interventions enhance virus-free generation of TRAC-replaced CAR T cells. Mol Ther—Methods Clin Dev. 2022:25(June):311-30.
16. Mueller K P. Piscopo N J, Forsberg M H, Saraspe L A, Das A, Russell B, et al. Production and characterization of virus-free, CRISPR-CAR T cells capable of inducing solid tumor regression. J Immunother cancer. 2022:10(9):1-22.
17. Madison B. Patil D, Richter M. Li X, Cranert S. Wang X, et al. Cas-CLOVER is a novel high-fidelity nulcease for safe and robust generation of TSCM-enriched allogeneic CAR-T cells. Submiss. 2021; 29(September):329.
18. Eyquem J, Mansilla-Soto J, Giavridis T, van der Stegen S J C, Hamieh M, Cunanan K M, et al. Targeting a CAR to the TRAC locus with CRISPR/Cas9 enhances tumour rejection. Nature. 2017; 543(7643):113-7.
19. Dai X, Park J J, Du Y, Kim H R, Wang G, Errami Y, et al. One-step generation of modular CAR-T cells with AAV-Cpf1. Nat Methods. 2019:16(3):247-54.
20. Roth T L, Puig-Saus C, Yu R. Shifrut E, Canevale J, Li P J, et al. Reprogramming human T cell function and specificity with non-viral genome targeting. Nature. 2018:559(7714):405-9.
21. Jing R. Jiao P, Chen J. Meng X, Wu X, Duan Y, et al. Cas9-Cleavage Sequences in Size-Reduced Plasmids Enhance Nonviral Genome Targeting of CARs in Primary Human T Cells. Small Methods. 2021 Jul. 1:5(7):2100071.
22. Zhang J, Hu Y, Yang J. Li W, Zhang M, Wang Q. et al. Non-viral, specifically targeted CAR-T cells achieve high safety and efficacy in B-NHL. Nature. 2022; 609(March 2021).
23. Shy B R, Vykunta V S, Ha A, Talbot A, Roth T L, Nguyen D N, et al. High-yield genome engineering in primary cells using a hybrid ssDNA repair template and small-molecule cocktails. Nat Biotechnol. 2022.
24. Schrank B, Gautier J. Assembling nuclear domains: Lessons from DNA repair. J Cell Biol. 2019 Jul. 19; 218(8):2444-55.
25. Suzuki K, Tsunekawa Y. Hemandez-Benitez R, Wu J, Zhu J, Kim E J, et al. In vivo genome editing via CRISPR/Cas9 mediated homology-independent targeted integration. Nature. 2016; 540(7631):144-9.
26. Chang H H Y, Pannunzio N R, Adachi N, Lieber M R. Non-homologous DNA end joining and alternative pathways to double-strand break repair. Nat Rev Mol Cell Biol. 2017; 18(8):495-506.
27. Kelly J J, Saee-Marand M, Nystrom N N, Evans M M. Chen Y, Martinez F M, et al. Safe harbor-targeted CRISPR-Cas9 homology-independent targeted integration for multimodality reporter gene-based cell tracking. Sci Adv. 2021; 7(4).
28. He X, Tan C, Wang F, Wang Y, Zhou R, Cui D. et al. Knock-in of large reporter genes in human cells via CRISPR/Cas9-induced homology-dependent and independent DNA repair. Nucleic Acids Res. 2016 May 19:44(9):e85-c85.
29. Tomabene P. Ferla R, Llado-Santaeularia M, Centrulo M, Dell'Anno M, Esposito F, et al. Therapeutic homology-independent targeted integration in retina and liver. Nat Commun. 2022; 13(1):1963.
30. Hong S-A, Sco J H. Wi S. Jung E S, Yu J, Hwang G-H, et al. In vivo gene editing via homology-independent targeted integration for adrenoleukodystrophy treatment. Mol Ther. 2022:30(1):119-29.
31. Suzuki K, Izpisua Belmonte J C. In vivo genome editing via the HITI method as a tool for gene therapy. J Hum Genet. 2018:63(2):157-64.
32. Shapiro J, Iancu O. Jacobi A M, McNeill M S, Turk R, Rettig G R, et al. Increasing CRISPR Efficiency and Measuring Its Specificity in HSPCs Using a Clinically Relevant System. Mol Ther—Methods Clin Dev. 2020; 17(June):1097-107.
33. de Vree P J P, de Wit E, Yilmaz M. van de Heijning M, Klous P. Verstegen MJAM, et al. Targeted sequencing by proximity ligation for comprehensive variant detection and local haplotyping. Nat Biotechnol. 2014; 32(10):1019-25.
34. Williams J. Vector Design for Improved DNA Vaccine Efficacy, Safety and Production. Vaccines. 2013; 1(3):225-49.
35. Cames A E, Williams J A. Plasmid Fermentation Process for DNA Immunization Applications BT—DNA Vaccines: Methods and Protocols. In: Rinaldi M, Fioretti D, Iurescia S, editors. New York, NY: Springer New York: 2014. p. 197-217.
36. Suschak J J, Dupuy L C, Shoemaker C J, Six C. Kwilas S A, Spik K W, et al. Nanoplasmid Vectors Co-expressing Innate Immune Agonists Enhance DNA Vaccines for Venezuelan Equine Encephalitis Virus and Ebola Virus. Mol Ther—Methods Clin Dev. 2020:17(June):810-21.
37. Ren J, Liu X, Fang C, Jiang S, June C H, Zhao Y. Multiplex Genome Editing to Generate Universal CART Cells Resistant to PD1 Inhibition. Clin Cancer Res. 2017 Apr. 30; 23(9):2255-66.
38. Xu H. Wang B. Ono M, Kagita A, Fujii K. Sasakawa N, et al. Targeted Disruption of HLA Genes via CRISPR-Cas9 Generates iPSCs with Enhanced Immune Compatibility. Cell Stem Cell. 2019; 24(4):566-578.e7.
39. Bister A, Ibach T, Haist C, Gerhorst G, Smorra D, Soldierer M, et al. Optimized NGFR-derived hinges for rapid and efficient enrichment and detection of CAR T cells in vitro and in vivo. Mol Ther—Oncolytics. 2022:26:120-34.
40. Spiegel J Y, Patel S, Muffly L, Hossain N M, Oak J, Baird J H, et al. CAR T cells with dual targeting of CD19 and CD22 in adult patients with recurrent or refractory B cell malignancies: a phase 1 trial. Nat Med. 2021; 27(8):1419-31.
41. Labanieh L, Majzner R G, Klysz D, Sotillo E, Fisher C J, Vilches-Moure J G, et al. Enhanced safety and efficacy of protease-regulated CAR-T cell receptors. Cell. 2022:185(10):1745-1763.e22.
42. Baird J H, Frank M J, Craig J, Patel S, Spiegel J Y, Sahaf B, et al. CD22-directed CAR T-cell therapy induces complete remissions in CD19-directed CAR-refractory large B-cell lymphoma. Blood. 2021; 137(17):2321-5.
43. Cradick T J, Qiu P, Lee C M, Fine E J, Bao G. COSMID: A web-based tool for identifying and validating CRISPR/Cas off-target sites. Mol Ther—Nucleic Acids. 2014:3(12):e214.
44. Stemmer M, Thumberger T, del Sol Keyer M, Wittbrodt J. Mateo J L. CCTop: An Intuitive, Flexible and Reliable CRISPR/Cas9 Target Prediction Tool. PLoS One. 2015 Apr. 24; 10(4):e0124633.
45. Wiebking V, Patterson J O, Martin R, Chanda M K, Lee C M, Srifa W. et al. Metabolic engineering generates a transgene-free safety switch for cell therapy. Nat Biotechnol. 2020; 38(12):1441-50.
46. Kurgan G, Turk R, Li H, Roberts N, Rettig G R, Jacobi A M, et al. CRISPAltRations: a validated cloud-based approach for interrogation of double-strand break repair mediated by CRISPR genome editing. Mol Ther—Methods Clin Dev. 2021:21(June):478-91.
47. Oh S A, Senger K, Madireddi S. Akhmetzyanova I, Ishizuka I E, Tarighat S, et al. High-efficiency nonviral CRISPR/Cas9-mediated gene editing of human T cells using plasmid donor DNA. J Exp Med. 2022 Apr. 22; 219(5):e20211530.
48. Chen C C, Kass E M, Yen W F, Ludwig T. Moynahan M E, Chaudhuri J, et al. ATM loss leads to synthetic lethality in BRCA1 BRCT mutant mice associated with exacerbated defects in homology-directed repair. Proc Natl Acad Sci USA. 2017; 114(29):7665-70.
49. Jonnalagadda M, Brown C E, Chang W C, Ostberg J R, Forman S J, Jensen M C. Efficient selection of genetically modified human T cells using methotrexate-resistant human dihydrofolate reductase. Gene Ther. 2013; 20(8):853-60.
50. Ercikan-Abali E A, Mineishi S, Tong Y, Nakahara S, Waltham M C, Banerjee D, et al. Active site-directed double mutants of dihydrofolate reductase. Cancer Res. 1996 September; 56(18):4142-5.
51. Paul B, Ibarra G S R, Hubbard N, Einhaus T, Astrakhan A. Rawlings D J, et al. Efficient Enrichment of Gene-Modified Primary T Cells via CCR5-Targeted Integration of Mutant Dihydrofolate Reductase. Mol Ther—Methods Clin Dev. 2018:9(June):347-57.
52. Foy S P, Jacoby K, Bota D A, Hunter T. Pan Z. Stawiski E, et al. Non-viral precision T cell receptor replacement for personalized cell therapy. Nature. 2023; 615(7953):687-96.
53. Nguyen D N, Roth T L, Li P J, Chen P A, Apathy R, Mamedov M R, et al. Polymer-stabilized Cas9 nanoparticles and modified repair templates increase genome editing efficiency. Nat Biotechnol. 2020; 38(1):44-9.
54. Smith T T, Stephan S B, Moffett H F, McKnight L E, Ji W, Reiman D, et al. In situ programming of leukaemia-specific t cells using synthetic DNA nanocarriers. Nat Nanotechnol. 2017; 12(8):813-22.
55. Ghassemi S, Durgin J S, Nunez-Cruz S, Patel J, Leferovich J, Pinzone M, et al. Rapid manufacturing of non-activated potent CAR T cells. Nat Biomed Eng. 2022; 6(2):118-28.

### EXEMPLARY EMBODIMENTS

Exemplary embodiments provided in accordance with the presently disclosed subject matter include, but are not limited to, the claims and the following embodiments:

Embodiment 1. A method for enriching for T cells that express a chimeric antigen receptor (CAR) inserted at a target genomic locus following homology-independent targeted insertion (HITI), comprising:
transfecting a plurality of T cells with
(a) a linear nucleic acid molecule comprising:
(i) an expression cassette comprising a promoter operably linked to a sequence encoding a CAR and a sequence encoding a selectable marker; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i); and
(b) a guide RNA-CRISPR/Cas9 ribonucleoprotein (RNP):
wherein the nucleic acid molecule is inserted into a target site in the genome of the T cell via non-homologous end joining (NHEJ); and
isolating T cells that express the selectable marker, wherein the number of CAR+ T cells is increased compared to T cells that do not express the selectable marker.

Embodiment 2. The method of embodiment 1, wherein the nucleic acid molecule does not comprise sequences that are homologous to genomic sequences at the target genomic locus.

Embodiment 3. The method of embodiment 1 or 2, wherein the single PAM is located 5′ of the expression cassette of (i).

Embodiment 4. The method of any one of embodiments 1 to 3, wherein the nucleic acid molecule is inserted on the positive or negative strand of the target genomic locus.

Embodiment 5. The method of any one of embodiments 1 to 4, wherein the nucleic acid molecule comprises a plasmid having a plasmid backbone of less than 500 bp.

Embodiment 6. The method of any one of embodiments 1 to 5, wherein the plasmid comprises an insert size of at least 2000 bp.

Embodiment 7. The method of embodiment 6, wherein the plasmid comprises an insert size between 2000 and 5000 bp.

Embodiment 8. The method of any one of embodiments 1 to 7, wherein the promoter is an EF-1α promoter or EF-1α short (EFS) promoter.

Embodiment 9. The method of any one of embodiments 1 to 8, wherein the promoter comprises SEQ ID NO:1.

Embodiment 10. The method of any one of embodiments 1 to 9, wherein the T cells are activated T cells.

Embodiment 11. The method of any one of embodiments 1 to 10, wherein the T cells are activated prior to transfection with the nucleic acid molecule.

Embodiment 12. The method of any one of embodiments 1 to 11, wherein the T cells are incubated with an inhibitor of homology directed repair (HDR).

Embodiment 13. The method of embodiment 12, wherein the inhibitor of HDR is selected from the group consisting of an ATM/ATR kinase inhibitor, a Chk1/Chk2 inhibitor, a BRCA1 inhibitor, a Rad51 inhibitor, and combinations thereof.

Embodiment 14. The method of embodiment 13, wherein the inhibitor of HDR is an ATM/ATR kinase inhibitor.

Embodiment 15. The method of any one of embodiments 1 to 14, wherein the selectable marker is a protein expressed on the cell surface and expression of the selectable marker is detected by contacting an antibody to the selectable marker.

Embodiment 16. The method of embodiment 15, wherein the protein expressed on the cell surface is tEGFR or tNGFR.

Embodiment 17. The method of any one of embodiments 1 to 14, wherein the selectable marker is a protein that confers resistance to a drug or compound.

Embodiment 18. The method of embodiment 17, wherein the selectable marker is DHFR-FS, and the T cells are cultured with methotrexate (MTX) for a period of time to reduce the number of cells that do not express DHFR-FS.

Embodiment 19. The method of embodiment 18, wherein the T cells are cultured with MTX beginning on day 1 after transfection until day 5 after transfection.

Embodiment 20. The method of embodiment 18, wherein the T cells are cultured with MTX beginning on day 1 after transfection until day 4 after transfection, followed by culturing the T cells without MTX for 3 to 7 days.

Embodiment 21. The method of embodiment 18, wherein the T cells are cultured with MTX beginning on day 5 after transfection until day 12 after transfection.

Embodiment 22. The method of any one of embodiments 18 to 21, wherein the frequency of CAR+ T cells is equal to or greater than 70%.

Embodiment 23. The method of embodiment 19, wherein the T cells are cultured with MTX beginning on day 1 after transfection until day 5 after transfection, and are then cultured without MTX until day 12 after transfection, and the yield of CAR+ T cells at day 12 is similar to the yield of CAR+ T cells cultured with MTX beginning on day 5 after transfection until day 12 after transfection.

Embodiment 24. The method of any one of embodiments 1 to 23, wherein the transfection comprises electroporation.

Embodiment 25. The method of any one of embodiments 1 to 24, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

Embodiment 26. The method of any one of embodiments 1 to 25, wherein the nucleic acid molecule is integrated into the T cell receptor alpha constant (TRAC), beta-2-microglobulin (B2M), or adeno-associated virus integration site 1 (AAVS1) genomic locus.

Embodiment 27. The method of any one of embodiments 1 to 26, wherein the T cell is a primary human T cell.

Embodiment 28. The method of any one of embodiments 1 to 27, wherein the T cells are unstimulated T cells.

Embodiment 29. A method for treating a tumor in a subject, comprising administering to the subject an effective amount of a genetically modified T cell comprising:
a nucleic acid molecule comprising:
(i) an expression cassette comprising a promoter operably linked to a sequence encoding a chimeric antigen receptor (CAR) and a sequence encoding a selectable marker; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i);
wherein the nucleic acid molecule is inserted into a target site in the genome of the T cell via non-homologous end joining (NHEJ), thereby treating the tumor.

Embodiment 30. The method of embodiment 29, wherein the nucleic acid molecule does not comprise sequences that are homologous to genomic sequences at the target genomic locus.

Embodiment 31. The method of embodiment 29 or 30, wherein the single PAM is located 5′ of the expression cassette of (i).

Embodiment 32. The method of any one of embodiments 29 to 31, wherein the nucleic acid molecule comprises a plasmid having a plasmid backbone of less than 500 bp.

Embodiment 33. The method of any one of embodiments 29 to 32, wherein the plasmid comprises an insert size between 2000 and 5000 bp.

Embodiment 34. The method of any one of embodiments 29 to 33, wherein the promoter is an EF-1α promoter or EF-1α short (EFS) promoter.

Embodiment 35. The method of any one of embodiments 29 to 34, wherein the promoter comprises SEQ ID NO:1.

Embodiment 36. The method of any one of embodiments 29 to 35, wherein the selectable marker is DHFR-FS, and the T cells are cultured with methotrexate (MTX) for a period of time to reduce the number of cells that do not express DHFR-FS.

Embodiment 37. The method of embodiment 36, wherein the T cells are cultured with MTX beginning on day 1 after transfection until day 4 after transfection, followed by culturing the T cells without MTX for 3 to 7 days.

Embodiment 38. The method of embodiment 36, wherein the T cells are cultured with MTX beginning on day 7 after transfection until day 14 after transfection.

Embodiment 39. The method of any one of embodiments 29 to 35, wherein the selectable marker is a protein expressed on the cell surface and expression of the selectable marker is detected by contacting an antibody to the selectable marker.

Embodiment 40. The method of embodiment 39, wherein the protein expressed on the cell surface is tEGFR or tNGFR.

Embodiment 41. The method of any one of embodiments 29 to 40, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR. EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

Embodiment 42. The method of any one of embodiments 29 to 41, wherein the nucleic acid molecule is integrated into the T cell receptor alpha constant (TRAC), beta-2-microglobulin (B2M), or adeno-associated virus integration site 1 (AAVS1) genomic locus.

Embodiment 43. The method of any one of embodiments 29 to 42, wherein the T cells are activated prior to transfection with the nucleic acid molecule.

Embodiment 44. The method of any one of embodiments 29 to 43, wherein 1×10⁶ to 1×10⁹ CAR+ T cells are administered to the subject.

Embodiment 45. The method of any one of embodiments 29 to 43, wherein 0.1×10⁶ to 5×10⁶ CAR+ T cells/kg of the subject's weight are administered to the subject.

Embodiment 46. The method of any one of embodiments 29 to 45, wherein the CAR+ T cells are administered in one or more doses.

Embodiment 47. A genetically modified T cell comprising:
a nucleic acid molecule comprising:
(i) an expression cassette comprising a promoter operably linked to a sequence encoding a CAR and a sequence encoding DHFR-FS; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i).

Embodiment 48. The genetically modified T cell of embodiment 47, wherein the promoter is an EF-1α promoter or EF-1α short (EFS) promoter.

Embodiment 49. The genetically modified T cell of embodiment 48, wherein the promoter comprises SEQ ID NO: 1.

Embodiment 50. The genetically modified T cell of any one of embodiments 47 to 49, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR. EGFRvIII. FAP, CD33, CD123. PD-L1, IGF1R, CD4, CSPG4, B7-H4, NKG2D, CS1. CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

Embodiment 51. The genetically modified T cell of any one of embodiments 47 to 50, wherein the T cell further comprises a guide RNA and CRISPR/Cas9 nuclease.

Embodiment 52. The genetically modified T cell of any one of embodiments 47 to 51, wherein the nucleic acid molecule is integrated into the T cell receptor alpha constant (TRAC), beta-2-microglobulin (B2M), or adeno-associated virus integration site 1 (AAVS1) genomic locus.

Embodiment 53. The genetically modified T cell of any one of embodiments 47 to 52, wherein the T cell is a primary human T cell.

Embodiment 54. The genetically modified T cell of any one of embodiments 47 to 53, wherein the T cell is an activated T cell.

Embodiment 55. The genetically modified T cell of any one of embodiments 47 to 54, wherein the T cell is an unstimulated T cell.

Embodiment 56. A pharmaceutical composition comprising the genetically modified T cell of any one of embodiments 47 to 55.

It is understood that the examples and embodiments described herein are for illustrative purposes only and that various modifications or changes in light thereof will be suggested to persons skilled in the art and are to be included within the spirit and purview of this application and scope of the appended claims. All references, patent applications, patents and sequence accession numbers are hereby incorporated by reference in their entirety.

Informal Sequence Listing
EFS (SEQ ID NO: 1):
5′-gggcagagcgcacatcgcccacagtccccgagaagttggggggagg
ggtcggcaattgaaccggtgcctagagaaggtggcgcggggtaaactggg
aaagtgatgtcgtgtactggctccgcctttttcccgaggggggggagaac
cgtatataagtgcagtagtcgccgtgaacgttctttttcgcaacgggttt
gccgccagaacacag-3′
EFla (SEQ ID NO: 2):
5′-ggctccggtgcccgtcagtgggcagagcgcacatcgcccacagtcc
ccgagaagttggggggaggggtcggcaattgaaccggtgcctagagaagg
tggcgcggggtaaactgggaaagtgatgtcgtgtactggctccgcctttt
tcccgaggggggggagaaccgtatataagtgcagtagtcgccgtgaacgt
tctttttcgcaacgggtttgccgccagaacacaggtaagtgccgtgtgtg
gttcccgcgggcctggcctctttacgggttatggcccttgcgtgccttga
attacttccacctggctgcagtacgtgattcttgatcccgagcttcgggt
tggaagtgggtgggagagttcgaggccttgcgcttaaggagccccttcgc
ctcgtgcttgagttgaggcctggcctgggcgctggggccgccgcgtgcga
atctggtggcaccttcgcgcctgtctcgctgctttcgataagtctctagc
catttaaaatttttgatgacctgctgcgacgctttttttctggcaagata
gtcttgtaaatggggccaagatctgcacactggtatttcggtttttgggg
ccgcgggcggcgacggggcccgtgcgtcccagcgcacatgttcggcgagg
cggggcctgcgagcgcggccaccgagaatcggacgggggtagtctcaagc
tggccggcctgctctggtgcctggcctcgcgccgccgtgtatcgccccgc
cctgggcggcaaggctggcccggtcggcaccagttgcgtgagcggaaaga
tggccgcttcccggccctgctgcagggagctcaaaatggaggacgcggcg
ctcgggagagcggggggtgagtcacccacacaaaggaaaagggcctttcc
gtcctcagccgtcgcttcatgtgactccactgagtaccgggcgccgtcca
ggcacctcgattagttctcgtgcttttggagtacgtcgtctttaggttgg
ggggaggggttttatgcgatggagtttccccacactgagtgggtggagac
tgaagttaggccagcttggcacttgatgtaattctccttggaatttgccc
tttttgagtttggatcttggttcattctcaagcctcagacagtggttcaa
agtttttttttccatttcaggtgtcgtga-3′
HITI1c Knock-In template sequence; cut_site-EFS-
Signalpeptide-14g2a-CD8TM-4-1BB-
CD3z-STOP-bGH_ployA; (SEQ ID NO: 3):
ggtaccCCTATTCACCGATTTTGATTCCCGggcagagcgcacatcgccca
cagtccccgagaagttggggggaggggtcggcaattgaaccggtgcctag
agaaggtggcgcggggtaaactgggaaagtgatgtcgtgtactggctccg
cctttttcccgagggtgggggagaaccgtatataagtgcagtagtcgccg
tgaacgttctttttcgcaacgggtttgccgccagaacacagATGCTGCTG
CTCGTGACATCTCTGCTGCTGTGCGAGCTGCCCCACCCCGCCTTTCTGCT
GATCCCCGATATCCTGCTGACCCAGACCCCTCTGAGCCTGCCTGTGTCTC
TGGGCGATCAGGCCAGCATCAGCTGCAGATCCAGCCAGAGCCTGGTGCAC
CGGAACGGCAACACCTACCTGCACTGGTATCTGCAGAAGCCCGGCCAGAG
CCCCAAGCTGCTGATTCACAAGGTGTCCAACCGGTTCAGCGGCGTGCCCG
ACAGATTTTCTGGCAGCGGCTCCGGCACCGACTTCACCCTGAAGATCAGC
CGGGTGGAAGCCGAGGACCTGGGCGTGTACTTCTGCAGCCAGTCCACCCA
CGTGCCCCCCCTGACATTTGGCGCCGGAACAAAGCTGGAACTGAAGGGCA
GCACAAGCGGCAGCGGCAAGCCTGGATCTGGCGAGGGAAGCACCAAGGGC
GAAGTGAAGCTGCAGCAGAGCGGCCCCTCTCTGGTGGAACCTGGCGCCTC
TGTGATGATCTCCTGCAAGGCCAGCGGCAGCTCCTTCACCGGCTACAACA
TGAACTGGGTGCGCCAGAACATCGGCAAGAGCCTGGAATGGATCGGCGCC
ATCGACCCCTACTACGGCGGCACCAGCTACAACCAGAAGTTCAAGGGCAG
AGCCACCCTGACCGTGGACAAGAGCAGCTCCACCGCCTACATGCACCTGA
AGTCCCTGACCAGCGAGGACAGCGCCGTGTACTACTGCGTGTCCGGCATG
GAATACTGGGGCCAGGGCACAAGCGTGACCGTGTCCTCTCCAGCGCCGCG
ACCACCAACACCGGCGCCCACCATCGCGTCGCAGCCCCTGTCCCTGCGCC
CAGAGGCGTGCCGGCCAGCGGCGGGGGGCGCAGTGCACACGAGGGGGCTG
GACTTCGCCTGTGATATCTACATCTGGGCGCCCTTGGCCGGGACTTGTGG
GGTCCTTCTCCTGTCACTGGTTATCACCCTTTACTGCAAACGGGGCAGAA
AGAAACTCCTGTATATATTCAAACAACCATTTATGAGACCAGTACAAACT
ACTCAAGAGGAAGATGGCTGTAGCTGCCGATTTCCAGAAGAAGAAGAAGG
AGGATGTGAACTGAGAGTGAAGTTCAGCAGGAGCGCAGACGCCCCCGCGT
ACCAGCAGGGCCAGAACCAGCTCTATAACGAGCTCAATCTAGGACGAAGA
GAGGAGTACGATGTTTTGGACAAGAGACGTGGCCGGGACCCTGAGATGGG
GGGAAAGCCGAGAAGGAAGAACCCTCAGGAAGGCCTGTACAATGAACTGC
AGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGC
CACCAAGGACACCTACGACGCCCTTCACATGCAGGCCCTGCCCCCTCGCT
AATAAGAATTCTAACTAGAGCTCGCTGATCAGCCTCGACTGTGCCTTCTA
GTTGCCAGCCATCTGTTGTTTGCCCCTCCCCCGTGCCTTCCTTGACCCTG
GAAGGTGCCACTCCCACTGTCCTTTCCTAATAAAATGAGGAAATTGCATC
GCATTGTCTGAGTAGGTGTCATTCTATTCTGGGGGGTGGGGTGGGGCAGG
ACAGCAAGGGGGAGGATTGGGAAGAGAATAGCAGGCATGCTGGGGAGCGG
CCGCGTTAAgctagc
HITI2c Knock-In template sequence; cut_site-EFS-
Signalpeptide-14g2a-CD8TM-4-1BB-
CD3z-STOP-bGH_ployA-cut_site; (SEQ ID NO: 4):
ggtaccCCTATTCACCGATTTTGATTCCCgggcagagcgcacatcgccca
cagtccccgagaagttggggggaggggtcggcaattgaaccggtgcctag
agaaggtggcgggggtaaactgggaaagtgatgtgtgtactggctccgcc
tttttcccgagggtgggggagaaccgtatataagtgcagtagtcgccgtg
aacgttctttttcgcaacgggtttgccgccagaacacagatgctgctgct
cgtgacatctctgctgctgtgcgagctgccccaccccgcctttctgctga
tccccgatatcctgctgacccagacccctctgagcctgcctgtgtctctg
ggcgatcaggccagcatcagctgcagatccagccagagcctggtgcaccg
gaacggcaacacctacctgcactggtatctgcagaagcccggccagagcc
ccaagctgctgattcacaaggtgtccaaccggttcagcggcgtgcccgac
agattttctggcagcggctccggcaccgacttcaccctgaagatcagccg
ggtggaagccgaggacctgggcgtgtacttctgcagccagtccacccacg
tgccccccctgacatttggcgccggaacaaagctggaactgaagggcagc
acaagcggcagcggcaagcctggatctggcgagggaagcaccaagggcga
agtgaagctgcagcagagcggcccctctctggtggaacctggcgcctctg
tgatgatctcctgcaaggccagcggcagctccttcaccggctacaacatg
aactgggtgcgccagaacatcggcaagagcctggaatggatcggcgccat
cgacccctactacggcggcaccagctacaaccagaagttcaagggcagag
ccaccctgaccgtggacaagagcagctccaccgcctacatgcacctgaag
tccctgaccagcgaggacagcgccgtgtactactgcgtgtccggcatgga
atactggggccagggcacaagcgtgaccgtgtcctctccagcgccgcgac
caccaacaccggcgcccaccatcgcgtcgcagcccctgtccctgcgccca
gaggcgtgccggccagcggggggggcgcagtgcacacgagggggctggac
ttcgcctgtgatatctacatctgggcgcccttggccgggacttgtggggt
ccttctcctgtcactggttatcaccctttactgcaaacggggcagaaaga
aactcctgtatatattcaaacaaccatttatgagaccagtacaaactact
caagaggaagatggctgtagctgccgatttccagaagaagaagaaggagg
atgtgaactgagagtgaagttcagcaggagcgcagacgcccccgcgtacc
agcagggccagaaccagctctataacgagctcaatctaggacgaagagag
gagtacgatgttttggacaagagacgtggccgggaccctgagatgggggg
aaagccgagaaggaagaaccctcaggaaggcctgtacaatgaactgcaga
aagataagatggcggaggcctacagtgagattgggatgaaaggcgagcgc
cggaggggcaaggggcacgatggcctttaccagggtctcagtacagccac
caaggacacctacgacgcccttcacatgcaggccctgccccctcgctaat
aagaattctaactagagctcgctgatcagcctcgactgtgccttctagtt
gccagccatctgtttgtttgcccctcccccgtgccttccttgaccctgga
aggtgccactcccactgtcctttcctaataaaatgaggaaattgcatcgc
attgtctgagtaggtgtcattctattctggggggggggggggcaggacag
caagggggaggattgggaagagaatagcaggcatgctggggagcggccgc
gttaaCCTATTCACCGATTTTGATTCCCgctagc
HDR2c; cut site-LHA-bGH polyA-STOP-CD3z-4-1BB-
CD8TM-14g2a-Signalpeptide-EFS-
RHA-cut_site; (SEQ ID NO: 5):
ggtaccCCTATTCACCGATTTTGATTCCCTATTAAATAAAAGAATAAGCA
GTATTATTAAGTAGCCCTGCATTTCAGGTTTCCTTGAGTGGCAGGCCAGG
CCTGGCCGTGAACGTTCACTGAAATCATGGCCTCTTGGCCAAGATTGATA
GCTTGTGCCTGTCCCTGAGTCCCAGTCCATCACGAGCAGCTGGTTTCTAA
GATGCTATTTCCCGTATAAAGCATGAGACCGTGACTTGCCAGCCCCACAG
AGCCCCGCCCTTGTCCATCACTGGCATCTGGACTCCAGCCTGGGTTGGGG
CAAAGAGGGAAATGAGATCATGTCCTAACCCTGATCCTCTTGTCCCACAG
ATATCCAGAACCCTGACCCTGCCGTGTACCAGCTGAGAGACTCTAAATCC
AGTGACAAGTCTGTCTGCCTttaacgcggccgctccccagcatgcctgct
attctcttcccaatcctcccccttgctgtcctgccccaccccacccccca
gaatagaatgacacctactcagacaatgcgatgcaatttcctcattttat
taggaaaggacagtgggagtggcaccttccagggtcaaggaaggcacggg
ggaggggcaaacaacagatggctggcaactagaaggcacagtcgaggctg
atcagcgagctctagttagaattcttattagcgagggggcagggcctgca
tgtgaagggcgtcgtaggtgtccttggtggctgtactgagaccctggtaa
aggccatcgtgccccttgcccctccggcgctcgcctttcatcccaatctc
actgtaggcctccgccatcttatctttctgcagttcattgtacaggcctt
cctgagggttcttccttctcggctttccccccatctcagggtcccggcca
cgtctcttgtccaaaacatcgtactcctctcttcgtcctagattgagctc
gttatagagctggttctggccctgctggtacgcgggggcgtctgcgctcc
tgctgaacttcactctcagttcacatcctccttcttcttcttctggaaat
cggcagctacagccatcttcctcttgagtagtttgtactggtctcataaa
tggttgtttgaatatatacaggagtttctttctgccccgtttgcagtaaa
gggtgataaccagtgacaggagaaggaccccacaagtcccggccaagggc
gcccagatgtagatatcacaggcgaagtccagccccctcgtgtgcactgc
gccccccgccgctggccggcacgcctctgggcgcagggacaggggctgcg
acgcgatggtgggcgccggtgttggtggtcgcggcgctggagaggacacg
gtcacgcttgtgccctggccccagtattccatgccggacacgcagtagta
cacggcgctgtcctcgctggtcagggacttcaggtgcatgtaggcggtgg
agctgctcttgtccacggtcagggtggctctgcccttgaacttctggttg
tagctggtgccgccgtagtaggggtcgatggcgccgatccattccaggct
cttgcgatgttctggcgcacccagttcatgttgtagccggtgaaggagct
gccgctggccttgcaggagatcatcacagaggcgccaggttccaccagag
aggggccgctctgctgcagcttcacttcgcccttggtgcttccctcgcca
gatccaggcttgccgctgccgcttgtgctgcccttcagttccagctttgt
tccggcgccaaatgtcagggggggcacgtgggtggactggctgcagaagt
acacgcccaggtcctcggcttccacccggctgatcttcagggtgaagtcg
gtgccggagccgctgccagaaaatctgtcgggcacgccgctgaaccggtt
ggacaccttgtgaatcagcagcttggggctctggccgggcttctgcagat
accagtgcaggtaggtgttgccgttccggtgcaccaggctctggctggat
ctgcagctgatgctggcctgatcgcccagagacacaggcaggctcagagg
ggtctgggtcagcaggatatcggggatcagcagaaaggcggggggggcag
ctcgcacagcagcagagatgtcacgagcagcagcatctgtgttctggcgg
caaacccgttgcgaaaaagaacgttcacggcgactactgcacttatatac
ggttctcccccaccctcgggaaaaaggcggagccagtacacgacatcact
ttcccagtttaccccgcgccaccttctctaggcaccggttcaattgccga
cccctccccccaacttctcggggactgtgggcgatgtgcgctctgcccTT
TGATTCTCAAACAAATGTGTCACAAAGTAAGGATTCTGATGTGTATATCA
CAGACAAAACTGTGCTAGACATGAGGTCTATGGACTTCAAGAGCAACAGT
GCTGTGGCCTGGAGCAACAAATCTGACTTTGCATGTGCAAACGCCTTCAA
CAACAGCATTATTCCAGAAGACACCTTCTTCCCCAGCCCAGGTAAGGGCA
GCTTTGGTGCCTTCGCAGGCTGTTTCCTTGCTTCAGGAATGGCCAGGTTC
TGCCCAGAGCTCTGGTCAATGATGTCTAAAACTCCTCTGATTGGTGGTCT
CGGCCTTATCCATTGCCACCAAAACCCTCTTTTTACTAAGAAACAGTGAG
CCTTGTTCTGGCAGTCCAGAGAATGACACCTATTCACCGATTTTGATTCC
Cgctagc
GD2-CAR + DHFR; cut_site-EFS-Signalpeptide-14g2a-
CD8TM-4-1BB-CD3z-P2A-DHFR-
FS-STOP-bGH_ployA; (SEQ ID NO: 6):
ggtaccCCTATTCACCGATTTTGATTCCCGggcagagcgcacatcgccca
cagtccccgagaagttggggggaggggtcggcaattgaaccggtgcctag
agaaggtggcgcggggtaaactgggaaagtgatgtcgtgtactggctccg
cctttttcccgagggtgggggagaaccgtatataagtgcagtagtcgccg
tgaacgttctttttcgcaacgggtttgccgccagaacacagATGCTGCTG
CTCGTGACATCTCTGCTGCTGTGCGAGCTGCCCCACCCCGCCTTTCTGCT
GATCCCCGATATCCTGCTGACCCAGACCCCTCTGAGCCTGCCTGTGTCTC
TGGGCGATCAGGCCAGCATCAGCTGCAGATCCAGCCAGAGCCTGGTGCAC
CGGAACGGCAACACCTACCTGCACTGGTATCTGCAGAAGCCCGGCCAGAG
CCCCAAGCTGCTGATTCACAAGGTGTCCAACCGGTTCAGCGGCGTGCCCG
ACAGATTTTCTGGCAGCGGCTCCGGCACCGACTTCACCCTGAAGATCAGC
CGGGTGGAAGCCGAGGACCTGGGCGTGTACTTCTGCAGCCAGTCCACCCA
CGTGCCCCCCCTGACATTTGGCGCCGGAACAAAGCTGGAACTGAAGGGCA
GCACAAGCGGCAGCGGCAAGCCTGGATCTGGCGAGGGAAGCACCAAGGGC
GAAGTGAAGCTGCAGCAGAGCGGCCCCTCTCTGGTGGAACCTGGCGCCTC
TGTGATGATCTCCTGCAAGGCCAGCGGCAGCTCCTTCACCGGCTACAACA
TGAACTGGGTGCGCCAGAACATCGGCAAGAGCCTGGAATGGATCGGCGCC
ATCGACCCCTACTACGGCGGCACCAGCTACAACCAGAAGTTCAAGGGCAG
AGCCACCCTGACCGTGGACAAGAGCAGCTCCACCGCCTACATGCACCTGA
AGTCCCTGACCAGCGAGGACAGCGCCGTGTACTACTGCGTGTCCGGCATG
GAATACTGGGGCCAGGGCACAAGCGTGACCGTGTCCTCTCCAGCGCCGCG
ACCACCAACACCGGCGCCCACCATCGCGTCGCAGCCCCTGTCCCTGCGCC
CAGAGGCGTGCCGGCCAGCGGCGGGGGGCGCAGTGCACACGAGGGGGCTG
GACTTCGCCTGTGATATCTACATCTGGGCGCCCTTGGCCGGGACTTGTGG
GGTCCTTCTCCTGTCACTGGTTATCACCCTTTACTGCAAACGGGGCAGAA
AGAAACTCCTGTATATATTCAAACAACCATTTATGAGACCAGTACAAACT
ACTCAAGAGGAAGATGGCTGTAGCTGCCGATTTCCAGAAGAAGAAGAAGG
AGGATGTGAACTGAGAGTGAAGTTCAGCAGGAGCGCAGACGCCCCCGCGT
ACCAGCAGGGCCAGAACCAGCTCTATAACGAGCTCAATCTAGGACGAAGA
GAGGAGTACGATGTTTTGGACAAGAGACGTGGCCGGGACCCTGAGATGGG
GGGAAAGCCGAGAAGGAAGAACCCTCAGGAAGGCCTGTACAATGAACTGC
AGAAAGATAAGATGGCGGAGGCCTACAGTGAGATTGGGATGAAAGGCGAG
CGCCGGAGGGGCAAGGGGCACGATGGCCTTTACCAGGGTCTCAGTACAGC
CACCAAGGACACCTACGACGCCCTTCACATGCAGGCCCTGCCCCCTCGCG
AAGGCCGAGGGAGCCTGCTGACATGTGGCGATGTGGAGGAAAACCCAGGA
CCAATGGTGGGCAGCCTGAACTGCATCGTGGCCGTGAGCCAGAACATGGG
CATCGGCAAGAACGGCGACTTCCCCTGGCCCCCCCTGAGAAACGAGAGCA
GATACTTCCAGAGAATGACCACCACCAGCAGCGTGGAGGGCAAGCAGAAC
CTGGTGATCATGGGCAAGAAGACCTGGTTCAGCATCCCCGAGAAGAACAG
ACCCCTGAAGGGCAGAATCAACCTGGTGCTGAGCAGAGAGCTGAAGGAGC
CCCCCCAGGGCGCCCACTTCCTGAGCAGAAGCCTGGACGACGCCCTGAAG
CTGACCGAGCAGCCCGAGCTGGCCAACAAGGTGGACATGGTGTGGATCGT
GGGCGGCAGCAGCGTGTACAAGGAGGCCATGAACCACCCCGGCCACCTGA
AGCTGTTCGTGACCAGAATCATGCAGGACTTCGAGAGCGACACCTTCTTC
CCCGAGATCGACCTGGAGAAGTACAAGCTGCTGCCCGAGTACCCCGGCGT
GCTGAGCGACGTGCAGGAGGAGAAGGGCATCAAGTACAAGCTGGAGGTGT
ACGAGAAGAACGACTAATAAGAATTCTAACTAGAGCTCGCTGATCAGCCT
CGACTGTGCCTTCTAGTTGCCAGCCATCTGTTGTTTGCCCCTCCCCCGTG
CCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGG
GGGGGTGGGGCAGGACAGCAAGGGGGAGGATTGGGAAGAGAATAGCAGGC
ATGCTGGGGAGCGGCCGCGTTAAgctagc
GD2-CAR + (EGFR; cut_site-EFS-Signalpeptide-14g2a-
CD8TM-4-1BB-CD3z-P2A-Signalpeptide-tEGFR-STOP-bGH_
ployA; (SEQ ID NO: 7):
ggtaccCCTATTCACCGATTTTGATTCCCgggcagagcgcacatcgccca
cagtccccgagaagttggggggaggggtcggcaattgaaccggtgcctag
agaaggtggcgcggggtaaactgggaaagtgatgtcgtgtactggctccg
cctttttcccgaggggggggagaaccgtatataagtgcagtagtcgccgt
gaacgttctttttcgcaacgggtttgccgccagaacacagatgctgctgc
tcgtgacatctctgctgctgtgcgagctgccccaccccgcctttctgctg
atccccgatatcctgctgacccagacccctctgagcctgcctgtgtctct
gggcgatcaggccagcatcagctgcagatccagccagagcctggtgcacc
ggaacggcaacacctacctgcactggtatctgcagaagcccggccagagc
cccaagctgctgattcacaaggtgtccaaccggttcagcggcgtgcccga
cagattttctggcagcggctccggcaccgacttcaccctgaagatcagcc
gggtggaagccgaggacctgggcgtgtacttctgcagccagtccacccac
gtgccccccctgacatttggcgccggaacaaagctggaactgaagggcag
cacaagcggcagcggcaagcctggatctggcgagggaagcaccaagggcg
aagtgaagctgcagcagagcggcccctctctggtggaacctggcgcctct
gtgatgatctcctgcaaggccagcggcagctccttcaccggctacaacat
gaactgggtgcgccagaacatcggcaagagcctggaatggatcggcgcca
tcgacccctactacggcggcaccagctacaaccagaagttcaagggcaga
gccaccctgaccgtggacaagagcagctccaccgcctacatgcacctgaa
gtccctgaccagcgaggacagcgccgtgtactactgcgtgtccggcatgg
aatactggggccagggcacaagcgtgaccgtgtcctctccagcgccgcga
ccaccaacaccggcgcccaccatcgcgtcgcagcccctgtccctgcgccc
agaggcgtgccggccagcggggggggcgcagtgcacacgagggggctgga
cttcgcctgtgatatctacatctgggcgcccttggccgggacttgtgggg
tccttctcctgtcactggttatcaccctttactgcaaacggggcagaaag
aaactcctgtatatattcaaacaaccatttatgagaccagtacaaactac
tcaagaggaagatggctgtagctgccgatttccagaagaagaagaaggag
gatgtgaactgagagtgaagttcagcaggagcgcagacgcccccgcgtac
cagcagggccagaaccagctctataacgagctcaatctaggacgaagaga
ggagtacgatgttttggacaagagacgtggccgggaccctgagatggggg
gaaagccgagaaggaagaaccctcaggaaggcctgtacaatgaactgcag
aaagataagatggcggaggcctacagtgagattgggatgaaaggcgagcg
ccggaggggcaaggggcacgatggcctttaccagggtctcagtacagcca
ccaaggacacctacgacgcccttcacatgcaggccctgccccctcgcgaa
ggccgagggagcctgctgacatgtggcgatgtggaggaaaacccaggacc
aatgcttctcctggtgacaagccttctgctctgtgagttaccacacccag
cattcctcctgatcccacgcaaagtgtgtaacggaataggtattggtgaa
tttaaagactcactctccataaatgctacgaatattaaacacttcaaaaa
ctgcacctccatcagtggcgatctccacatcctgccggtggcatttaggg
gtgactccttcacacatactcctcctctggatccacaggaactggatatt
ctgaaaaccgtaaaggaaatcacagggtttttgctgattcaggcttggcc
tgaaaacaggacggacctccatgcctttgagaacctagaaatcatacgcg
gcaggaccaagcaacatggtcagttttctcttgcagtcgtcagcctgaac
ataacatccttgggattacgctccctcaaggagataagtgatggagatgt
gataatttcaggaaacaaaaatttgtgctatgcaaatacaataaactgga
aaaaactgtttgggacctccggtcagaaaaccaaaattataagcaacaga
ggtgaaaacagctgcaaggccacaggccaggtctgccatgccttgtgctc
ccccgagggctgctggggcccggagcccagggactgcgtctcttgccgga
atgtcagccgaggcagggaatgcgtggacaagtgcaaccttctggagggt
gagccaagggagtttgtggagaactctgagtgcatacagtgccacccaga
gtgcctgcctcaggccatgaacatcacctgcacaggacggggaccagaca
actgtatccagtgtgcccactacattgacggcccccactgcgtcaagacc
tgcccggcaggagtcatgggagaaaacaacaccctggtctggaagtacgc
agacgccggccatgtgtgccacctgtgccatccaaactgcacctacggat
gcactgggccaggtcttgaaggctgtccaacgaatgggcctaagatcccg
tccatcgccactgggatggtgggggccctcctcttgctgctggtggtggc
cctggggatcggcctcttctaataagaattctaactagagctcgctgatc
agcctcgactgtgccttctagttgccagccatctgttgtttgcccctccc
ccgtgccttccttgaccctggaaggtgccactcccactgtcctttcctaa
taaaatgaggaaattgcatcgcattgtctgagtaggtgtcattctattct
ggggggtggggggggcaggacagcaagggggaggattgggaagagaatag
caggcatgctggggagcggccgcgttaagctagc
GD2-CAR + tNGFR; cut_site-EFS-Signalpeptide-
14g2a-CD8TM-4-1BB-CD3z-P2A-
Signalpeptide-tNGFR-STOP-bGH_ployA; (SEQ ID NO: 8)
ggtaccCCTATTCACCGATTTTGATTCCCgggcagagcgcacatcgccca
cagtccccgagaagttggggggaggggtcggcaattgaaccggtgcctag
agaaggtggcgcggggtaaactgggaaagtgatgtcgtgtactggctccg
cctttttcccgaggggggggagaaccgtatataagtgcagtagtcgcgtg
aacgttctttttcgcaacgggtttgccgccagaacacagatgctgctgct
cgtgacatctctgctgctgtgcgagctgccccaccccgcctttctgctga
tccccgatatcctgctgacccagacccctctgagcctgcctgtgtctctg
ggcgatcaggccagcatcagctgcagatccagccagagcctggtgcaccg
gaacggcaacacctacctgcactggtatctgcagaagcccggccagagcc
ccaagctgctgattcacaaggtgtccaaccggttcagcggcgtgcccgac
agattttctggcagcggctccggcaccgacttcaccctgaagatcagccg
ggtggaagccgaggacctgggcgtgtacttctgcagccagtccacccacg
tgccccccctgacatttggcgccggaacaaagctggaactgaagggcagc
acaagcggcagcggcaagcctggatctggcgagggaagcaccaaggggaa
gtgaagctgcagcagagcggcccctctctggtggaacctggcgcctctgt
gatgatctcctgcaaggccagcggcagctccttcaccggctacaacatga
actgggtgcgccagaacatcggcaagagcctggaatggatcggcgccatc
gacccctactacggcggcaccagctacaaccagaagttcaagggcagagc
caccctgaccgtggacaagagcagctccaccgcctacatgcacctgaagt
ccctgaccagcgaggacagcgccgtgtactactgcgtgtccggcatggaa
tactggggccagggcacaagcgtgaccgtgtcctctccagcgccgcgacc
accaacaccggcgcccaccatcgcgtcgcagcccctgtccctgcgcccag
aggcgtgccggccagcggggggggcgcagtgcacacgagggggctggact
tcgcctgtgatatctacatctgggcgcccttggccgggacttgtggggtc
cttctcctgtcactggttatcaccctttactgcaaacggggcagaaagaa
actcctgtatatattcaaacaaccatttatgagaccagtacaaactactc
aagaggaagatggctgtagctgccgatttccagaagaagaagaaggagga
tgtgaactgagagtgaagttcagcaggagcgcagacgcccccgcgtacca
gcagggccagaaccagctctataacgagctcaatctaggacgaagagagg
agtacgatgttttggacaagagacgtggccgggaccctgagatgggggga
aagccgagaaggaagaaccctcaggaaggcctgtacaatgaactgcagaa
agataagatggcggaggcctacagtgagattgggatgaaaggcgagcgcc
ggaggggcaaggggcacgatggcctttaccagggtctcagtacagccacc
aaggacacctacgacgcccttcacatgcaggccctgccccctcgcgaagg
ccgagggagcctgctgacatgtggcgatgtggaggaaaacccaggaccaa
tgggggcaggtgccaccggccgcgcaatggacgggccgcgcctgctgctg
ttgctgcttctgggggtgtcccttggaggtgccaaggaggcatgccccac
aggcctgtacacacacagcggtgagtgctgcaaagcctgcaacctgggcg
agggtgtggcccagccttgtggagccaaccagaccgtgtgtgagccctgc
ctggacagcgtgacgttctccgacgtggtgagcgcgaccgagccgtgcaa
gccgtgcaccgagtgcgtggggctccagagcatgtcggcgccgtgcgtgg
aggccgacgacgccgtgtgccgctgcgcctacggctactaccaggatgag
acgactgggcgctgcgaggcgtgccgcgtgtgcgagggggctcgggcctc
gtgttctcctgccaggacaagcagaacaccgtgtgcgaggagtgccccga
cggcacgtattccgacgaggccaaccacgtggacccgtgcctgccctgca
ccgtgtgcgaggacaccgagcgccagctccgcgagtgcacacgctgggcc
gacgccgagtgcgaggagatccctggccgttggattacacggtccacacc
cccagagggctcggacagcacagcccccagcacccaggagcctgaggcac
ctccagaacaagacctcatagccagcacggtggcaggtgtggtgaccaca
gtgatgggcagctcccagcccgtggtgacccgaggcaccaccgacaacct
catccctgtctattgctccatcctggctgctgtggttgtgggccttgtgg
cctacatagccttcaagaggtgataagaattctaactagagctcgctgat
cagcctcgactgtgccttctagttgccagccatctgttgtttgcccctcc
cccgtgccttccttgaccctggaaggtgccactcccactgtcctttccta
ataaaatgaggaaattgcatcgcattgtctgagtaggtgtcattctattc
tggggggtggggggggcaggacagcaagggggaggattgggaagagaata
gcaggcatgctggggagcggccgcgttaagctagc
HDR2c-TRAC-promotor; cut_site-LHA-SA-T2A-Signal
peptide14g2a-CD8TM-4-1BB-
CD3z-STOP-bGH ployA-RHA-cut site; (SEQ ID NO: 9)
ggtaccCCCACAGATATCCAGAACCCTGAcctttttcccatgcctgcctt
tactctgccagagttatattgctggggttttgaagaagatcctattaaat
aaaagaataagcagtattattaagtagccctgcatttcaggtttccttga
gtggcaggccaggcctggccgtgaacgttcactgaaatcatggcctcttg
gccaagattgatagcttgtgcctgtccctgagtcccagtccatcacgagc
agctggtttctaagatgctatttcccgtataaagcatgagaccgtgactt
gccagccccacagagccccgcccttgtccatcactggcatctggactcca
gcctgggttggggcaaagagggaaatgagatcatgtcctaaccctgatcc
tcttgtcccacaagcttctgacctcttctcttcctcccacagggcctcga
gagatctggcagcggaGAAGGCCGAGGGAGCCTGCTGACATGTGGCGATG
TGGAGGAAAACCCAGGACCACCATGGATGCTGCTGCTCGTGACATCTCTG
CTGCTGTGCGAGCTGCCCCACCCCGCCTTTCTGCTGATCCCCGATATCCT
GCTGACCCAGACCCCTCTGAGCCTGCCTGTGTCTCTGGGCGATCAGGCCA
GCATCAGCTGCAGATCCAGCCAGAGCCTGGTGCACCGGAACGGCAACACC
TACCTGCACTGGTATCTGCAGAAGCCCGGCCAGAGCCCCAAGCTGCTGAT
TCACAAGGTGTCCAACCGGTTCAGCGGCGTGCCCGACAGATTTTCTGGCA
GCGGCTCCGGCACCGACTTCACCCTGAAGATCAGCCGGGTGGAAGCCGAG
GACCTGGGCGTGTACTTCTGCAGCCAGTCCACCCACGTGCCCCCCCTGAC
ATTTGGCGCCGGAACAAAGCTGGAACTGAAGGGCAGCACAAGCGGCAGCG
GCAAGCCTGGATCTGGCGAGGGAAGCACCAAGGGCGAAGTGAAGCTGCAG
CAGAGCGGCCCCTCTCTGGTGGAACCTGGCGCCTCTGTGATGATCTCCTG
CAAGGCCAGCGGCAGCTCCTTCACCGGCTACAACATGAACTGGGTGCGCC
AGAACATCGGCAAGAGCCTGGAATGGATCGGCGCCATCGACCCCTACTAC
GGCGGCACCAGCTACAACCAGAAGTTCAAGGGCAGAGCCACCCTGACCGT
GGACAAGAGCAGCTCCACCGCCTACATGCACCTGAAGTCCCTGACCAGCG
AGGACAGCGCCGTGTACTACTGCGTGTCCGGCATGGAATACTGGGGCCAG
GGCACAAGCGTGACCGTGTCCTCTGCGGCCGCAACCACGACGCCAGCGCC
GCGACCACCAACACCGGCGCCCACCATCGCGTCGCAGCCCCTGTCCCTGC
GCCCAGAGGCGTGCCGGCCAGCGGCGGGGGGCGCAGTGCACACGAGGGGG
CTGGACTTCGCCTGTGATATCTACATCTGGGCGCCCTTGGCCGGGACTTG
TGGGGTCCTTCTCCTGTCACTGGTTATCACCCTTTACTGCAAACGGGGCA
GAAAGAAACTCCTGTATATATTCAAACAACCATTTATGAGACCAGTACAA
ACTACTCAAGAGGAAGATGGCTGTAGCTGCCGATTTCCAGAAGAAGAAGA
AGGAGGATGTGAACTGAGAGTGAAGTTCAGCAGGAGCGCAGACGCCCCCG
CGTACCAGCAGGGCCAGAACCAGCTCTATAACGAGCTCAATCTAGGACGA
AGAGAGGAGTACGATGTTTTGGACAAGAGACGTGGCCGGGACCCTGAGAT
GGGGGGAAAGCCGAGAAGGAAGAACCCTCAGGAAGGCCTGTACAATGAAC
TGCAGAAAGATAAGATGGCGGAGGCCTACAGTGAGATTGGGATGAAAGGC
GAGCGCCGGAGGGGCAAGGGGCACGATGGCCTTTACCAGGGTCTCAGTAC
AGCCACCAAGGACACCTACGACGCCCTTCACATGCAGGCCCTGCCCCCTC
GCTAAacgcgtgaattcactcctcaggtgcaggctgcctatcagaaggtg
gtggctggtgtggccaatgccctggctcacaaataccactgagatctttt
tccctctgccaaaaattatggggacatcatgaagccccttgagcatctga
cttctggctaataaaggaaatttattttcattgcaatagtgtgttggaat
tttttgtgtctctcactcggaaggacatatgggagggcaaatcatttaaa
acatcagaatgagtatttggtttagagtttggcaacatatgcccatatgc
tggctgccatgaacaaaggttggctataaagaggtcatcagtatatgaaa
cagccccctgctgtccattccttattccatagaaaagccttgacttgagg
ttagattttttttatattttgttttgtgttatttttttctttaacatccc
taaaattttccttacatgttttactagccagatttttcctcctctcctga
ctactcccagtcatagctgtccctcttctcttatggagatccctcgacct
gcagcccaagcttggcgtaatcatggtcatagctgtgATATCCAGAACCC
TGACCCTGCCGTGTACCAGCTGAGAGACTCTAAATCCAGTGACAAGTCTG
TCTGCCTATTCACCGATTTTGATTCTCAAACAAATGTGTCACAAAGTAAG
GATTCTGATGTGTATATCACAGACAAAACTGTGCTAGACATGAGGTCTAT
GGACTTCAAGAGCAACAGTGCTGTGGCCTGGAGCAACAAATCTGACTTTG
CATGTGCAAACGCCTTCAACAACAGCATTATTCCAGAAGACACCTTCTTC
CCCAGCCCAGgtaagggcagctttggtgccttcgcaggctgtttccttgc
ttcaggaatggccaggttctgcccagagctctggtcaatgatgtctaaaa
ctcctctgattggtggtctcggccttaCCCACAGATATCCAGAACCCTGA
gctagc
HDR2c-EFS-promotor; cut_site-LHA-bGH polyA-STOP-
CD3z-4-1BB-CD8TM-14g2a-
Signalpeptide-EFS-RHA-cut site; (SEQ ID NO: 10):
ggtaccCCCACAGATATCCAGAACCCTGAcctttttcccatgcctgcctt
tactctgccagagttatattgctggggttttgaagaagatcctattaaat
aaaagaataagcagtattattaagtagccctgcatttcaggtttccttga
gtggcaggccaggcctggccgtgaacgttcactgaaatcatggcctcttg
gccaagattgatagcttgtgcctgtccctgagtcccagtccatcacgagc
agctggtttctaagatgctatttcccgtataaagcatgagaccgtgactt
gccagccccacagagccccgcccttgtccatcactggcatctggactcca
gcctgggttggggcaaagagggaaatgagatcatgtcctaaccctgatcc
tcttgtcccacaTTAACGCGGCCGCTCCCCAGCATGCCTGCTATTCTCTT
CCCAATCCTCCCCCTTGCTGTCCTGCCCCACCCCACCCCCCAGAATAGAA
TGACACCTACTCAGACAATGCGATGCAATTTCCTCATTTTATTAGGAAAG
GACAGTGGGAGTGGCACCTTCCAGGGTCAAGGAAGGCACGGGGGAGGGGC
AAACAACAGATGGCTGGCAACTAGAAGGCACAGTCGAGGCTGATCAGCGA
GCTCTAGTTAGAATTCTTATTAGCGAGGGGGCAGGGCCTGCATGTGAAGG
GCGTCGTAGGTGTCCTTGGTGGCTGTACTGAGACCCTGGTAAAGGCCATC
GTGCCCCTTGCCCCTCCGGCGCTCGCCTTTCATCCCAATCTCACTGTAGG
CCTCCGCCATCTTATCTTTCTGCAGTTCATTGTACAGGCCTTCCTGAGGG
TTCTTCCTTCTCGGCTTTCCCCCCATCTCAGGGTCCCGGCCACGTCTCTT
GTCCAAAACATCGTACTCCTCTCTTCGTCCTAGATTGAGCTCGTTATAGA
GCTGGTTCTGGCCCTGCTGGTACGCGGGGGCGTCTGCGCTCCTGCTGAAC
TTCACTCTCAGTTCACATCCTCCTTCTTCTTCTTCTGGAAATCGGCAGCT
ACAGCCATCTTCCTCTTGAGTAGTTTGTACTGGTCTCATAAATGGITGTT
TGAATATATACAGGAGTTTCTTTCTGCCCCGTTTGCAGTAAAGGGTGATA
ACCAGTGACAGGAGAAGGACCCCACAAGTCCCGGCCAAGGGCGCCCAGAT
GTAGATATCACAGGCGAAGTCCAGCCCCCTCGTGTGCACTGCGCCCCCCG
CCGCTGGCCGGCACGCCTCTGGGCGCAGGGACAGGGGCTGCGACGCGATG
GTGGGCGCCGGTGTTGGTGGTCGCGGCGCTGGAGAGGACACGGTCACGCT
TGTGCCCTGGCCCCAGTATTCCATGCCGGACACGCAGTAGTACACGGCGC
TGTCCTCGCTGGTCAGGGACTTCAGGTGCATGTAGGCGGTGGAGCTGCTC
TTGTCCACGGTCAGGGTGGCTCTGCCCTTGAACTTCTGGTTGTAGCTGGT
GCCGCCGTAGTAGGGGTCGATGGCGCCGATCCATTCCAGGCTCTTGCCGA
TGTTCTGGCGCACCCAGTTCATGTTGTAGCCGGTGAAGGAGCTGCCGCTG
GCCTTGCAGGAGATCATCACAGAGGCGCCAGGTTCCACCAGAGAGGGGCC
GCTCTGCTGCAGCTTCACTTCGCCCTTGGTGCTTCCCTCGCCAGATCCAG
GCTTGCCGCTGCCGCTTGTGCTGCCCTTCAGTTCCAGCTTTGTTCCGGCG
CCAAATGTCAGGGGGGGCACGTGGGTGGACTGGCTGCAGAAGTACACGCC
CAGGTCCTCGGCTTCCACCCGGCTGATCTTCAGGGTGAAGTCGGTGCCGG
AGCCGCTGCCAGAAAATCTGTCGGGCACGCCGCTGAACCGGTTGGACACC
TTGTGAATCAGCAGCTTGGGGCTCTGGCCGGGCTTCTGCAGATACCAGTG
CAGGTAGGTGTTGCCGTTCCGGTGCACCAGGCTCTGGCTGGATCTGCAGC
TGATGCTGGCCTGATCGCCCAGAGACACAGGCAGGCTCAGAGGGGTCTGG
GTCAGCAGGATATCGGGGATCAGCAGAAAGGCGGGGTGGGGCAGCTCGCA
CAGCAGCAGAGATGTCACGAGCAGCAGCATGGTGGCctgtgttctggcgg
caaacccgttgcgaaaaagaacgttcacggcgactactgcacttatatac
ggttctcccccaccctcgggaaaaaggcggagccagtacacgacatcact
ttcccagtttaccccgcgccaccttctctaggcaccggttcaattgccga
cccctccccccaacttctcggggactgtgggcgatgtgcgctctgcccgA
TATCCAGAACCCTGACCCTGCCGTGTACCAGCTGAGAGACTCTAAATCCA
GTGACAAGTCTGTCTGCCTATTCACCGATTTTGATTCTCAAACAAATGTG
TCACAAAGTAAGGATTCTGATGTGTATATCACAGACAAAACTGTGCTAGA
CATGAGGTCTATGGACTTCAAGAGCAACAGTGCTGTGGCCTGGAGCAACA
AATCTGACTTTGCATGTGCAAACGCCTTCAACAACAGCATTATTCCAGAA
GACACCTTCTTCCCCAGCCCAGgtaagggcagctttggtgccttcgcagg
ctgtttccttgcttcaggaatggccaggttctgcccagagctctggtcaa
tgatgtctaaaactcctctgattggtggtctcggccttaCCCACAGATAT
CCAGAACCCTGAgctagc.
HDROc; cut site-LHA-bGH polyA-STOP-CD3z-4-1BB-
CD8TM-14g2a-Signalpeptide-EFS-
RHA-cut site; (SEQ ID NO: 11):
ggtaccCCTATTCACCGATTTTGATTCCCcctttttcccatgcctgcctt
tactctgccagagttatattgctggggttttgaagaagatcctattaaat
aaaagaataagcagtattattaagtagccctgcatttcaggtttccttga
gtggcaggccaggcctggccgtgaacgttcactgaaatcatggcctcttg
gccaagattgatagcttgtgcctgtccctgagtcccagtccatcacgagc
agctggtttctaagatgctatttcccgtataaagcatgagaccgtgactt
gccagccccacagagccccgcccttgtccatcactggcatctggactcca
gcctgggttggggcaaagagggaaatgagatcatgtcctaaccctgatcc
tcttgtcccacattaacgcggccgctccccagcatgcctgctattctctt
cccaatcctcccccttgctgtcctgccccaccccaccccccagaatagaa
tgacacctactcagacaatgcgatgcaatttcctcattttattaggaaag
gacagtgggagtggcaccttccagggtcaaggaaggcacgggggaggggc
aaacaacagatggctggcaactagaaggcacagtcgaggctgatcagcga
gctctagttagaattcttattagcgagggggcagggcctgcatgtgaagg
gcgtcgtaggtgtccttggtggctgtactgagaccctggtaaaggccatc
gtgccccttgcccctccggcgctcgcctttcatcccaatctcactgtagg
cctccgccatcttatctttctgcagttcattgtacaggccttcctgaggg
ttcttccttctcggctttccccccatctcagggtcccggccacgtctctt
gtccaaaacatcgtactcctctcttcgtcctagattgagctcgttataga
gctggttctggccctgctggtacgcgggggcgtctgcgctcctgctgaac
ttcactctcagttcacatcctccttcttcttcttctggaaatcggcagct
acagccatcttcctcttgagtagtttgtactggtctcataaatggttgtt
tgaatatatacaggagtttctttctgccccgtttgcagtaaagggtgata
accagtgacaggagaaggaccccacaagtcccggccaagggcgcccagat
gtagatatcacaggcgaagtccagccccctcgtgtgcactgcgccccccg
ccgctggccggcacgcctctgggcgcagggacaggggctgcgacgcgatg
gtgggcgccggtgttggtggtcgcggcgctggagaggacacggtcacgct
tgtgccctggccccagtattccatgccggacacgcagtagtacacggcgc
tgtcctcgctggtcagggacttcaggtgcatgtaggcggtggagctgctc
ttgtccacggtcagggtggctctgcccttgaacttctggttgtagctggt
gccgccgtagtaggggtcgatggcgccgatccattccaggctcttgccga
tgttctggcgcacccagttcatgttgtagccggtgaaggagctgccgctg
gccttgcaggagatcatcacagaggcgccaggttccaccagagaggggcc
gctctgctgcagcttcacttcgcccttggtgcttccctcgccagatccag
gcttgccgctgccgcttgtgtgcccttcagttccagctttgttccggcgc
caaatgtcagggggggcacgtgggtggactggctgcagaagtacacgccc
aggtcctcggcttccacccggctgatcttcagggtgaagtcggtgccgga
gccgctgccagaaaatctgtcgggcagccgctgaaccggttggacacctt
gtgaatcagcagcttggggctctggccgggcttctgcagataccagtgca
ggtaggtgttgccgttccggtgcaccaggctctggctggatctgcagctg
atgctggcctgatcgcccagagacacaggcaggctcagaggggtctgggt
cagcaggatatcggggatcagcagaaaggcggggggggcagctcgcacag
cagcagagatgtcacgagcagcagcatctgtgttctggcggcaaacccgt
tgcgaaaaagaacgttcacggcgactactgcacttatatacggttctccc
ccaccctcgggaaaaaggcggagccagtacacgacatcactttcccagtt
taccccgcgccaccttctctaggcaccggttcaattgccgacccctcccc
ccaacttctcggggactgtgggcgatgtgcgctctgcccgatatccagaa
ccctgaccctgccgtgtaccagctgagagactctaaatccagtgacaagt
ctgtctgcctattcaccgattttgattctcaaacaaatgtgtcacaaagt
aaggattctgatgtgtatatcacagacaaaactgtgctagacatgaggtc
tatggacttcaagagcaacagtgctgtggcctggagcaacaaatctgact
ttgcatgtgcaaacgccttcaacaacagcattattccagaagacaccttc
ttccccagcccaggtaagggcagctttggtgccttcgcaggctgtttcct
tgcttcaggaatggccaggttctgcccagagctctggtcaatgatgtcta
aaactcctctgattggtggtctcggccttaCCTATTCACCGATTTTGATT
CCCgctagc.
HITIIc-B2M; cut_site-EFS-Signalpeptide-14g2a-CD8TM-
4-1BB-CD3z-STOP-bGH_ployA;
(SEQ ID NO: 12):
ggtaccCCACGGAGCGAGACATCTCGGCCGggcagagcgcacatcgccca
cagtccccgagaagttggggggaggggtcggcaattgaaccggtgcctag
agaaggtggcgcggggtaaactgggaaagtgatgtcgtgtactggctccg
cctttttcccgaggggggggagaaccgtatataagtgcagtagtcgccgt
gaacgttctttttcgcaacgggtttgccgccagaacacagATGCTGCTGC
TCGTGACATCTCTGCTGCTGTGCGAGCTGCCCCACCCCGCCTTTCTGCTG
ATCCCCGATATCCTGCTGACCCAGACCCCTCTGAGCCTGCCTGTGTCTCT
GGGCGATCAGGCCAGCATCAGCTGCAGATCCAGCCAGAGCCTGGTGCACC
GGAACGGCAACACCTACCTGCACTGGTATCTGCAGAAGCCCGGCCAGAGC
CCCAAGCTGCTGATTCACAAGGTGTCCAACCGGTTCAGCGGCGTGCCCGA
CAGATTTTCTGGCAGCGGCTCCGGCACCGACTTCACCCTGAAGATCAGCC
GGGTGGAAGCCGAGGACCTGGGCGTGTACTTCTGCAGCCAGTCCACCCAC
GTGCCCCCCCTGACATTTGGCGCCGGAACAAAGCTGGAACTGAAGGGCAG
CACAAGCGGCAGCGGCAAGCCTGGATCTGGCGAGGGAAGCACCAAGGGCG
AAGTGAAGCTGCAGCAGAGCGGCCCCTCTCTGGTGGAACCTGGCGCCTCT
GTGATGATCTCCTGCAAGGCCAGCGGCAGCTCCTTCACCGGCTACAACAT
GAACTGGGTGCGCCAGAACATCGGCAAGAGCCTGGAATGGATCGGCGCCA
TCGACCCCTACTACGGCGGCACCAGCTACAACCAGAAGTTCAAGGGCAGA
GCCACCCTGACCGTGGACAAGAGCAGCTCCACCGCCTACATGCACCTGAA
GTCCCTGACCAGCGAGGACAGCGCCGTGTACTACTGCGTGTCCGGCATGG
AATACTGGGGCCAGGGCACAAGCGTGACCGTGTCCTCTCCAGCGCCGCGA
CCACCAACACCGGCGCCCACCATCGCGTCGCAGCCCCTGTCCCTGCGCCC
AGAGGCGTGCCGGCCAGCGGCGGGGGGCGCAGTGCACACGAGGGGGCTGG
ACTTCGCCTGTGATATCTACATCTGGGCGCCCTTGGCCGGGACTTGTGGG
GTCCTTCTCCTGTCACTGGTTATCACCCTTTACTGCAAACGGGGCAGAAA
GAAACTCCTGTATATATTCAAACAACCATTTATGAGACCAGTACAAACTA
CTCAAGAGGAAGATGGCTGTAGCTGCCGATTTCCAGAAGAAGAAGAAGGA
GGATGTGAACTGAGAGTGAAGTTCAGCAGGAGCGCAGACGCCCCCGCGTA
CCAGCAGGGCCAGAACCAGCTCTATAACGAGCTCAATCTAGGACGAAGAG
AGGAGTACGATGTTTTGGACAAGAGACGTGGCCGGGACCCTGAGATGGGG
GGAAAGCCGAGAAGGAAGAACCCTCAGGAAGGCCTGTACAATGAACTGCA
GAAAGATAAGATGGCGGAGGCCTACAGTGAGATTGGGATGAAAGGCGAGC
GCCGGAGGGGCAAGGGGCACGATGGCCTTTACCAGGGTCTCAGTACAGCC
ACCAAGGACACCIACGACGCCCTTCACATGCAGGCCCTGCCCCCTCGCTA
ATAAGAATTCTAACTAGAGCTCGCTGATCAGCCTCGACTGTGCCTTCTAG
TTGCCAGCCATCTGTTGTTTGCCCCTCCCCCGTGCCTTCCTTGACCCTGG
AAGGTGCCACTCCCACTGTCCTTTCCTAATAAAATGAGGAAATTGCATCG
CATTGTCTGAGTAGGTGTCATTCTATTCTGGGGGGTGGGGTGGGGCAGGA
CAGCAAGGGGGAGGATTGGGAAGAGAATAGCAGGCATGCTGGGGAGCGGC
CGCGTTAAgctagc.
gRNA sequence & mismatch from genome underlined
(TRAC gene)(SEQ ID NO: 13):
GGGAATCAAAATCGGTGAAT
genomic target sequence (SEQ ID NO: 14):
GAGAATCAAAATCGGTGAAT

What is claimed is:

1. A method for enriching for T cells that express a chimeric antigen receptor (CAR) inserted at a target genomic locus following homology-independent targeted insertion (HITI), comprising:
transfecting a plurality of T cells with
(a) a linear nucleic acid molecule comprising:
(i) an expression cassette comprising a promoter operably linked to a sequence encoding a CAR and a sequence encoding a selectable marker; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i); and
(b) a guide RNA-CRISPR/Cas9 ribonucleoprotein (RNP);
wherein the nucleic acid molecule is inserted into a target site in the genome of the T cell via non-homologous end joining (NHEJ); and
isolating T cells that express the selectable marker, wherein the number of CAR+ T cells is increased compared to T cells that do not express the selectable marker.

2. The method of claim 1, wherein the nucleic acid molecule does not comprise sequences that are homologous to genomic sequences at the target genomic locus.

3. The method of claim 1 or 2, wherein the single PAM is located 5′ of the expression cassette of (i).

4. The method of claim 1, wherein the nucleic acid molecule is inserted on the positive or negative strand of the target genomic locus.

5. The method of claim 1, wherein the nucleic acid molecule comprises a plasmid having a plasmid backbone of less than 500 bp.

6. The method of claim 1, wherein the plasmid comprises an insert size of at least 2000 bp.

7. The method of claim 6, wherein the plasmid comprises an insert size between 2000 and 5000 bp.

8. The method of claim 1, wherein the promoter is an EF-1α promoter or EF-1α short (EFS) promoter.

9. The method of claim 1, wherein the promoter comprises SEQ ID NO:1.

10. The method of claim 1, wherein the T cells are activated T cells.

11. The method of claim 1, wherein the T cells are activated prior to transfection with the nucleic acid molecule.

12. The method of claim 1, wherein the T cells are incubated with an inhibitor of homology directed repair (HDR).

13. The method of claim 12, wherein the inhibitor of HDR is selected from the group consisting of an ATM/ATR kinase inhibitor, a Chk1/Chk2 inhibitor, a BRCA1 inhibitor, a Rad51 inhibitor, and combinations thereof.

14. The method of claim 13, wherein the inhibitor of HDR is an ATM/ATR kinase inhibitor.

15. The method of claim 1, wherein the selectable marker is a protein expressed on the cell surface and expression of the selectable marker is detected by contacting an antibody to the selectable marker.

16. The method of claim 15, wherein the protein expressed on the cell surface is tEGFR or tNGFR.

17. The method of claim 1, wherein the selectable marker is a protein that confers resistance to a drug or compound.

18. The method of claim 17, wherein the selectable marker is DHFR-FS, and the T cells are cultured with methotrexate (MTX) for a period of time to reduce the number of cells that do not express DHFR-FS.

19. The method of claim 18, wherein the T cells are cultured with MTX beginning on day 1 after transfection until day 5 after transfection.

20. The method of claim 18, wherein the T cells are cultured with MTX beginning on day 1 after transfection until day 4 after transfection, followed by culturing the T cells without MTX for 3 to 7 days.

21. The method of claim 18, wherein the T cells are cultured with MTX beginning on day 5 after transfection until day 12 after transfection.

22. The method of claim 18, wherein the frequency of CAR+ T cells is equal to or greater than 70%.

23. The method of claim 19, wherein the T cells T cells are cultured with MTX beginning on day 1 after transfection until day 5 after transfection, and are then cultured without MTX until day 12 after transfection, and the yield of CAR+ T cells at day 12 is similar to the yield of CAR+ T cells cultured with MTX beginning on day 5 after transfection until day 12 after transfection.

24. The method of claim 1, wherein the transfection comprises electroporation.

25. The method of claim 1, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

26. The method of claim 1, wherein the nucleic acid molecule is integrated into the T cell receptor alpha constant (TRAC), beta-2-microglobulin (B2M), or adeno-associated virus integration site 1 (AAVS1) genomic locus.

27. The method of claim 1, wherein the T cell is a primary human T cell.

28. The method of claim 1, wherein the T cells are unstimulated T cells.

29. A method for treating a tumor in a subject, comprising administering to the subject an effective amount of a genetically modified T cell comprising:
a nucleic acid molecule comprising:
(i) an expression cassette comprising a promoter operably linked to a sequence encoding a chimeric antigen receptor (CAR) and a sequence encoding a selectable marker; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i);
wherein the nucleic acid molecule is inserted into a target site in the genome of the T cell via non-homologous end joining (NHEJ), thereby treating the tumor.

30. The method of claim 29, wherein the nucleic acid molecule does not comprise sequences that are homologous to genomic sequences at the target genomic locus.

31. The method of claim 29 or 30, wherein the single PAM is located 5′ of the expression cassette of (i).

32. The method of claim 29, wherein the nucleic acid molecule comprises a plasmid having a plasmid backbone of less than 500 bp.

33. The method of claim 29, wherein the plasmid comprises an insert size between 2000 and 5000 bp.

34. The method of claim 29, wherein the promoter is an EF-1α promoter or EF-1α short (EFS) promoter.

35. The method of claim 29, wherein the promoter comprises SEQ ID NO:1.

36. The method of claim 29, wherein the selectable marker is DHFR-FS, and the T cells are cultured with methotrexate (MTX) for a period of time to reduce the number of cells that do not express DHFR-FS.

37. The method of claim 36, wherein the T cells are cultured with MTX beginning on day 1 after transfection until day 4 after transfection, followed by culturing the T cells without MTX for 3 to 7 days.

38. The method of claim 36, wherein the T cells are cultured with MTX beginning on day 7 after transfection until day 14 after transfection.

39. The method of claim 29, wherein the selectable marker is a protein expressed on the cell surface and expression of the selectable marker is detected by contacting an antibody to the selectable marker.

40. The method of claim 39, wherein the protein expressed on the cell surface is tEGFR or tNGFR.

41. The method of claim 29, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

42. The method of claim 29, wherein the nucleic acid molecule is integrated into the T cell receptor alpha constant (TRAC), beta-2-microglobulin (B2M), or adeno-associated virus integration site 1 (AAVS1) genomic locus.

43. The method of claim 29, wherein the T cells are activated prior to transfection with the nucleic acid molecule.

44. The method of claim 29, wherein 1×10⁶ to 1×10⁹ CAR+ T cells are administered to the subject.

45. The method of claim 29, wherein 0.1×10⁶ to 5×10⁶ CAR+ T cells/kg of the subject's weight are administered to the subject.

46. The method of claim 29, wherein the CAR+ T cells are administered in one or more doses.

47. A genetically modified T cell comprising:
a nucleic acid molecule comprising:
(i) an expression cassette comprising a promoter operably linked to a sequence encoding a CAR and a sequence encoding DHFR-FS; and
(ii) a single protospacer adjacent motif (PAM) sequence that binds a guide RNA-CRISPR/Cas9 ribonucleoprotein (RNP), wherein the PAM is located 5′ or 3′ of the expression cassette of (i).

48. The genetically modified T cell of claim 47, wherein the promoter is an EF-1α promoter or EF-1α short (EFS) promoter.

49. The genetically modified T cell of claim 48, wherein the promoter comprises SEQ ID NO:1.

50. The genetically modified T cell of claim 47, wherein the CAR binds to an antigen selected from the group consisting of Her-2, B7-H3, GPC2, GD2, CD19, CD20, CD22, MAGE, BAGE, CAGE, GAGE, HAGE, LAGE, PAGE, PRAME, NY-ESO-1, NY-SEO-1, tyrosinase, Melan-A/MART, gp100, TRP-1, TRP-2, CD30, EGFR, EGFRvIII, FAP, CD33, CD123, PD-L1, IGF1R, CD4, CSPG4, B7-H4, NKG2D, CS1, CD138, EpCAM, EBNA3C, GPA7, CD244, CA-125, ETA, CEA, CD52, MUC5AC, c-Met, FAB, WT-1, PSMA, AFP, BCMA, Mesothelin, GPC3, MUC1 and CTAG1B.

51. The genetically modified T cell of claim 47, wherein the T cell further comprises a guide RNA and CRISPR/Cas9 nuclease.

52. The genetically modified T cell of claim 47, wherein the nucleic acid molecule is integrated into the T cell receptor alpha constant (TRAC), beta-2-microglobulin (B2M), or adeno-associated virus integration site 1 (AAVS1) genomic locus.

53. The genetically modified T cell of claim 47, wherein the T cell is a primary human T cell.

54. The genetically modified T cell of claim 47, wherein the T cell is an activated T cell.

55. The genetically modified T cell of claim 47, wherein the T cell is an unstimulated T cell.

56. A pharmaceutical composition comprising the genetically modified T cell of claim 47.
